(19) United States
(12) Patent Application Publication
(10) Pub. No.: US 2003/0186817 A1
(43) Pub. Date: Oct. 2, 2003

(54) SUBSTITUTED 2,4-DIAMINO-1,3,5-TRIAZINES, PROCESSES FOR THEIR PREPARATION AND THEIR USE AS HERBICIDES AND PLANT GROWTH REGULATORS

(76) Inventors: JENS HOLLANDER, EICHWALDSTRASSE (DE); LOTHAR WILLMS, KONIGSTEINER (DE); JURGEN ZINDEL, KIRSCHSTRASSE (DE); KLEMENS MINN, ROSSERSTRASSE (DE); WOLFGANG GIENCKE, AM STEINBERG (DE); HERMANN BIERINGER, EICHENWEG (DE); CHRISTOPHER ROSINGER, AM HOCHFELD (DE); THOMAS AULER, KASTANIENWEG (DE)

Correspondence Address: FROMMER LAWRENCE & HAUG, 745 FIFTH AVENUE- 10TH FL., NEW YORK, NY, 10151, US

(21) Appl. No.: 09/344,603

(22) Filed: Jun. 25, 1999

(30) Foreign Application Priority Data
Jun. 26, 1998 (DE) 198 28 519.1

(51) Int. Cl. A01N 43/00; C07D 251/48

(52) U.S. Cl. 504/232; 544/204

(57) ABSTRACT

Substituted 2,4-diamino-1,3,5-triazines of the formula I and salts thereof, processes for their preparation and their use as herbicides and plant growth regulators are described.

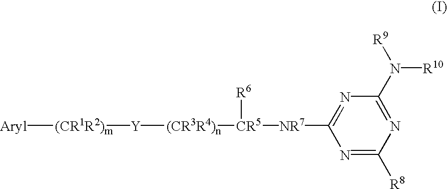

In the formula I, R¹ to R¹⁰ are various radicals, Y is a divalent unit, Aryl is an unsubstituted or substituted aromatic radical, m is from 0 to 5 and n is from 1 to 10.

The invention relates to the technical area of herbicides and plant growth regulators, in particular to that of herbicides for the selective control of broad-leaved weeds and weed grasses in crops of useful plants.

From various publications, it is already known that certain 2,4-diamino-1,3,5-triazines have herbicidal properties. Thus, WO 97/00254 describes 2,4-diamino-1,3,5-triazines which carry a phenyl radical attached to an amino group via an ethylene, oxyethylene, aminoethylene, thioethylene or propylene chain. WO 97/08156 discloses 2,4-diamino-1,3,5-triazines in which a phenyl radical is attached to an amino group via an alkylene, alkenylenemethyl or alkynylenemethyl chain. WO 98115537 discloses 2,4-diamino-1,3,5-triazines which carry an aromatic or heterocyclic radical attached via a propylene or oxyethylene chain to an amino group. Finally, WO 98115539 mentions 2,4-diamino-1,3,5-triazines which carry a benzyl, naphthylmethyl, heterocyclyl or heterocyclyloxy radical attached via an ethylene chain to an amino group.

However, in practice the use of the 2,4-diamino-1,3,5-triazines known from these publications frequently entails disadvantages. Thus, the herbicidal or plant growth-regulatory activity of the known compounds is not always sufficient or, where the herbicidal activity is sufficient, undesirable damage to the crop plants is observed.

It is an object of the present invention to provide herbicidal and plant growth-regulatory compounds which overcome the disadvantages known from the prior art.

The object is achieved by 2,4-amino-1,3,5-triazines of the formula I, if appropriate also in the form of their salts,

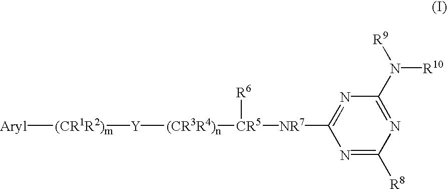

in which

Aryl is an unsubstituted or substituted mono- or bicyclic aromatic radical having 5 to 14 ring atoms, 1, 2, 3 or 4 of which may in each case independently of one another originate from the group consisting of oxygen, sulfur and nitrogen;

—Y— is a divalent unit from the group consisting of —O—, —S—, —NR¹¹—, —NR¹²CONR¹³—, —CO₂—, —OCO₂—, —OCONR¹⁴—, —SO—, —SO₂—, —SO₂O—, —OSO₂O—, —SO₂NR¹⁴—, —O—NR¹¹—, —NR′—NR″—, in which R′ and R″ independently of one another are as defined for R¹⁴, and —(Y′—CR^{a}R^{b}—CR^{c}R^{d}); —Y″, in which Y′ and Y″ independently of one another are O, S, NH or N[(C₁-C₄)alkyl], R^{a}, R^{b}, R^{c} and R^{d} in each case independently of one another are H or (C₁-C₄)alkyl and i is an integer from 1 to 5, preferably 1, or a trivalent unit of the formula —O—N═,

m is 0, 1, 2, 3, 4 or 5, preferably 0, 1, 2 or 3;

n is an integer from 1 to 10, preferably from 1 to 5, in particular 1, 2, 3 or 4, with the proviso that n is not 1 if m is zero and —Y— is —O—, —S—, —SO—, —SO₂— or —NR¹¹—,

R¹, R² in each case independently of one another are a radical from a group G1 which comprises hydrogen, (C₁-C₁₀)-alkyl, (C₂-C₈)-alkenyl, (C₂-C₈)-alkynyl, (C₁-C₁₀)-alkoxy, (C₃-C₈)-cycloalkyl, (C₃-C₈)-cycloalkoxy, aryl-(C₁-C₆)-alkyl and (C₃-C₈)-cycloalkyl-(C₁-C₆)-alkyl, where the cyclic moiety of the four last-mentioned radicals is in each case unsubstituted or substituted by one or more identical or different radicals from the group consisting of halogen, nitro, cyano, thiocyanato and —B—X¹, where —B— and X¹ are as defined below, and where the non-cyclic moiety of the eight last-mentioned radicals from the group G1 is in each case unsubstituted or substituted by one or more identical or different radicals from the group consisting of halogen, nitro, cyano, thiocyanato and —B—X², where X² is as defined below, and where the non-cyclic moiety of the radicals from the group G1 may in each case be interrupted by one or more identical or different heteroatoms from the group consisting of oxygen and sulfur, or

R¹ and R² of a (CR¹R²) group form, together with the carbon atom which carries them, a carbonyl group, a group CR¹⁵R¹⁶ or a 3- to 6-membered ring which may contain one or two identical or different heteroatoms from the group consisting of oxygen, nitrogen and sulfur and which is unsubstituted or substituted by one or more identical or different radicals from the group consisting of halogen, nitro, cyano, thiocyanato and —B—X¹, or

two R¹ of two directly adjacent or not directly adjacent (CR¹R²) groups together with the carbon atoms which carry or link them form an unsubstituted or substituted 3- to 6-membered ring which may contain one or two identical or different heteroatoms from the group consisting of oxygen, nitrogen and sulfur and which is unsubstituted or substituted by one or more identical or different radicals from the group consisting of halogen, nitro, cyano, thiocyanato and —B—X¹, or

two R¹ of two directly adjacent (CR¹R²) groups together with the bond between the carbon atoms of the groups form a double bond, or two R¹ and two R² of two directly adjacent (CR¹R²) groups together with the bond between the carbon atoms of the groups form a triple bond or else

R¹ is a linkage point for the double bond in the case where Y is a trivalent unit ═N—O— adjacent to a CR¹R² group,

R³, R⁴ in each case independently of one another are a radical of a group G2 which comprises hydrogen, (C₁-C₁₀)-alkyl, (C₂-C₈)-alkenyl, (C₂-C₈)-alkynyl, (C₁-C₁₀)-alkoxy, (C₁-C₁₀)-alkylthio, (C₁-C₁₀)-alkylsulfinyl, (C₁-C₁₀)-alkylsulfonyl, (C₃-C₈)-cycloalkyl, (C₃-C₈)-cycloalkoxy, aryl, aryl-(C₁-C₆)-alkyl, aryl-(C₁-C₆)-alkoxy, (C₃-C₈)-cycloalkyl-(C₁-C₆)-alkyl, (C₃-C₈)-cycloalkyl-(C₁-C₆)-alkoxy, (C₃-C₈)-cycloalkoxy-(C₁-C₆)-alkyl and (C₃-C₈)-cycloalkoxy-(C₁-C₆)-alkoxy, where the cyclic moiety of the nine last-mentioned radicals is in each case unsubstituted or substituted by one or more identical or different radicals from the group consisting of halogen, nitro, cyano, thiocyanato and —B—X¹, where —B— and X¹ are as defined below, and where the non-cyclic moiety of the sixteen last-mentioned radicals from the group G2 is in each case unsubstituted or substituted by one or more identical or different radicals from the group consisting of halogen, nitro, cyano, thiocyanato and —B—X², where X² is as defined below, and where the non-cyclic moiety of the radicals from the group G2 may in each case be interruped by one or more identical or different heteroatoms from the group consisting of oxygen and sulfur, or

R³ and R⁴ together with the carbon atom which carries them form a carbonyl group, a group CR¹⁵R¹⁶ or a 3- to 6-membered ring, which may contain one or two identical or different heteroatoms from the group consisting of oxygen, nitrogen and sulfur and which is unsubstituted or substituted by one or more identical or different radicals from the group consisting of halogen, nitro, cyano, thiocyanato and —B—X¹, or

two R³ of two directly adjacent or not directly adjacent (CR³R⁴) groups together with the carbon atoms which carry or link them form an unsubstituted or substituted 3- to 6-membered ring which may contain one or two identical or different heteroatoms from the group consisting of oxygen, nitrogen and sulfur and which is unsubstituted or substituted by one or more identical or different radicals from the group consisting of halogen, nitro, cyano, thiocyanato and —B—X¹; or else

two R³ of two directly adjacent (CR³R⁴) groups together with the bond between the carbon atoms of the groups form a double bond, or two R³ and two R⁴ of two directly adjacent (CR³R⁴) groups together with the bond between the carbon atoms of the groups form a triple bond, or else

R³ is a linkage point for the double bond in the case where Y is a trivalent unit —O—N═ adjacent to a CR³R⁴ group,

—B— is a direct bond or a divalent unit from the group consisting of —O—, —S—, —NR¹¹—, —NR¹²CONR¹³—, —CO₂—, —OCO₂—, —OCONR¹⁴—, —SO—, —SO₂—, —SO₂O—, —OSO₂O— and —SO₂NR¹⁴—;

X¹ is hydrogen, (C₁-C₈)-alkyl, (C₂-C₈)-alkenyl, (C₂-C₈)-alkynyl, (C₃-C₈)-cycloalkyl or heterocyclyl having 3 to 9 ring atoms, 1, 2 or 3 of which originate from the group consisting of nitrogen, oxygen and sulfur, and where the five last-mentioned radicals are unsubstituted or substituted by one or more identical or different halogen atoms;

X² is hydrogen or heterocyclyl having 3 to 9 ring atoms, 1, 2 or 3 of which originate from the group consisting of nitrogen, oxygen and sulfur, which is unsubstituted or substituted by one or more identical or different halogen atoms;

R⁵, R⁶ independently of one another are each a radical from the group G2, or

R³ and R⁵ of two directly adjacent or not directly adjacent (CR³R⁴) or (CR⁵R⁶) groups which with the carbon atoms which link them form an unsubstituted or substituted 3- to 6-membered ring which may contain one or two identical or different heteroatoms from the group consisting of oxygen, nitrogen and sulfur and which is unsubstituted or substituted by one or more identical or different radicals from the group consisting of halogen, nitro, cyano, thiocyanato and —B—X¹, or

R⁵ and R⁶ together with the carbon atom which carries them form a carbonyl group, a group CR¹⁵R¹⁶ or a 3- to 6-membered ring which may contain one or two identical or different heteroatoms from the group consisting of oxygen, nitrogen and sulfur and which is unsubstituted or substituted by one or more identical or different radicals from the group consisting of halogen, nitro, cyano, thiocyanato and —B—X¹, or

R⁶ is heterocyclyl,

R⁷ is hydrogen, amino, alkylcarbonyl, alkylamino or dialkylamino having in each case one to six carbon atoms in the alkyl radical, is an acyclic hydrocarbon or hydrocarbonoxy radical having in each case one to six carbon atoms, is a cyclic hydrocarbon or hydrocarbonoxy radical having in each case three to six carbon atoms or is heterocyclyl, heterocyclyloxy or heterocyclylamino having in each case three to six ring atoms and one to three hetero ring atoms from the group consisting of nitrogen, oxygen and sulfur, where each of the ten last-mentioned radicals is unsubstituted or substituted by one or more identical or different radicals from the group consisting of halogen, (C₁-C₄)-alkoxy, halogen-(C₁-C₄)-alkoxy, (C₁-C₄)-alkylthio, (C₂-C₄)-alkenyl, (C₂-C₄)-alkynyl, (C₂-C₄)-alkenyloxy, (C₂-C₄)-alkynyloxy, hydroxyl, amino, acylamino, alkylamino, dialkylamino, nitro, carboxyl, cyano, azido, (C₁-C₄)-alkoxycarbonyl, (C₁-C₄)-alkylcarbonyl, formyl, carbamoyl, mono- and di-(C₁-C₄)-alkylaminocarbonyl, (C₁-C₄)-alkylsulfinyl, halo-(C₁-C₄)-alkylsulfinyl, (C₁-C₄)-alkylsulfonyl, halo-(C₁-C₄)-alkylsulfonyl and, in the case of cyclic radicals, also (C₁-C₄)-alkyl and halo-(C₁-C₄)-alkyl,

R⁸ is (C₁-C₁₀)-alkyl, (C₂-C₈)-alkenyl, (C₂-C₈)-alkynyl, which are unsubstituted or substituted by one or more identical or different radicals from the group consisting of halogen, cyano, nitro, thiocyanato, hydroxyl, (C₁-C₄)-alkoxy, (C₁-C₄)-alkylthio, (C₁-C₄)-alkylsulfinyl, (C₁-C₄)-alkylsulfonyl, phenyl, (C₃-C₉)-cycloalkyl, (C₃-C₉)-cycloalkoxy, and is heterocyclyl having three to six ring atoms and one to three hetero ring atoms from the group consisting of oxygen, nitrogen and sulfur, which is unsubstituted or substituted by one or more identical or different radicals from the group consisting of halogen, amino, (C₁-C₄)-alkyl, (C₁-C₄)-alkoxy, halo-(C₁-C₄)-alkyl and halo-(C₁-C₄)-alkoxy, is (C₃-C₈)-cycloalkyl, (C₃-C₈)-cycloalkoxy or a heterocyclyl radical having three to six ring atoms, where these three last-mentioned radicals are unsubstituted or substituted by one or more identical or different radicals from the group consisting of halogen, nitro, cyano, thiocyanato, (C₁-C₄)-alkyl, (C₁-C₄)-alkoxy, halo-(C₁-C₄)-alkyl and halo-(C₁-C₄)-alkoxy,

R⁹, R¹⁰ independently of one another are each hydrogen, amino, (C₁-C₁₀)-alkylcarbonyl, (C₁-C₁₀)-alkylamino, di-[(C₁-C₁₀)-alkyl]amino, (C₁-C₁₀)-alkyl, (C₃-C₈)-cycloalkyl, (C₁-C₁₀)-alkoxy, (C₃-C₈)-cycloalkoxy, heterocyclyl, heterocyclyloxy or heterocyclylamino having in each case 3 to 6 ring atoms and 1 to 3 hetero ring atoms from the group consisting of oxygen, nitrogen and sulfur, where each of the ten last-mentioned radicals is unsubstituted or substituted, or

R⁹ and R¹⁰ together with the nitrogen atom which carries them form a heterocycle having a total of three to six ring atoms and from among these one to four hetero ring atoms, where the other hetero ring atoms which may be present in addition to the nitrogen atom that is present are selected from the group consisting of oxygen, nitrogen and sulfur and where this heterocycle is unsubstituted or substituted,

R¹¹ is hydrogen, amino, (C₁-C₁₀)-alkylamino, di-[(C₁-C₁₀)-alkyl]amino, (C₁-C₁₀)-alkyl, (C₃-C₈)-cycloalkyl, (C₃-C₈)-cycloalkyl-(C₁-C₆)-alkyl, (C₁-C₁₀)-alkoxy, (C₁-C₆)-alkoxy-(C₁-C₆)-alkoxy, (C₃-C₈)-cycloalkoxy, (C₁-C₁₀)-alkylcarbonyl, where the nine last-mentioned radicals are unsubstituted or substituted,

R¹², R¹³ independently of one another are each hydrogen, (C₁-C₁₀)-alkyl, (C₂-C₈)-alkenyl, (C₂-C₈)-alkynyl, phenyl, phenyl-(C₁-C₆)-alkyl, (C₃-C₈)-cycloalkyl, (C₃-C₈)-cycloalkyl-(C₁-C₆)-alkyl, where the cyclic moiety of the four last-mentioned radicals is in each case unsubstituted or substituted by one or more identical or different radicals from the group consisting of (C₁-C₄)-alkyl, halo-(C₁-C₄)-alkyl, (C₁-C₄)-alkoxy and halo-(C₁-C₄)-alkoxy, or

R¹² and R¹³ together with the N—CO—N group which carries them form a 5- to 8-membered ring which, in addition to the two abovementioned nitrogen atoms, may contain a further heteroatom from the group consisting of oxygen, nitrogen and sulfur and which is unsubstituted or substituted,

R¹⁴ is hydrogen or in each case unsubstituted or substituted (C₁-C₁₀)-alkyl or (C₃-C₁₀)-cycloalkyl,

R¹⁵, R¹⁶ independently of one another are each hydrogen, aryl, (C₁-C₁₀)-alkoxy, aryl-(C₁-C₆)-alkyl, (C₁-C₁₀)-alkyl, (C₁-C₁₀)-alkylthio, where the five last-mentioned radicals are unsubstituted or substituted, and where the aliphatic carbon skeleton of the three last-mentioned radicals may be interrupted by one or more identical or different heteroatoms from the group consisting of oxygen and sulfur, or

R¹⁵ and R¹⁶ together with the carbon atom which carries them form a 3- to 6-membered ring which may contain one or two identical or different heteroatoms from the group consisting of oxygen, nitrogen and sulfur and which is unsubstituted or substituted.

The term aryl is to be understood as meaning, for example, phenyl, naphthyl, biphenyl, tetrahydronaphthyl, indenyl, indanyl, pentalenyl, fluorenyl aziridinyl, azetidinyl, pyrrolyl, pyrazolyl, imidazolyl, triazolyl, tetrazolyl, thiazolyl, oxazolyl, thiadiazolyl, oxadiazolyl, pyridinyl, pyridazinyl, pyrimidinyl, pyrazinyl, triazinyl, tetrazinyl, oxazinyl, thiazinyl, thiadiazinyl, oxadiazinyl, dithiazinyl, thioxazinyl, indolyl, benzoxazolyl, benzothiazolyl, quinolinyl, isoquinolinyl, cinnolinyl, quinazolinyl, purinyl or naphthyridinyl. These aryl radicals may in principle, i.e. the structure of the ring system permitting, be attached to the remaining moiety of the molecule of the compound of the formula I at any position of the aryl radical.

If —Y— or —B— is an asymmetrical divalent unit, i.e. if there are two options for the attachment, in each case both options for the attachment of —Y— or —B— and the remainder of the molecule on the other hand are to be embraced by the formula I.

In the formula I and in all subsequent formulae, the carbon-containing radicals, such as alkyl, alkoxy, haloalkyl, haloalkoxy, alkylamino and alkylthio, and the corresponding radicals which are unsaturated and/or substituted in the carbon skeleton, such as alkenyl and alkynyl, can in each case be straight-chain or branched. Unless specifically mentioned otherwise, the lower carbon skeletons, for example those having 1 to 6 carbon atoms or in the case of unsaturated groups having 2 to 4 carbon atoms, are preferred for these radicals.

Alkyl radicals, including in composite meanings such as alkoxy, haloalkyl, etc., are, for example, methyl, ethyl, n- or i-propyl, n-, i-, t- or 2-butyl, pentyls, hexyls, such as n-hexyl, i-hexyl and 1,3-dimethylbutyl, heptyls, such as n-heptyl, 1-methylhexyl and 1,4-dimethylpentyl; alkenyl- and alkynyl radicals have the meaning of the possible unsaturated radicals which correspond to the alkyl radicals; alkenyl, is, for example, allyl, 1-methylprop-2-en-1-yl, 2-methylprop-2-en-1-yl, but-2-en-1-yl, but-3-en-1-yl, 1-methylbut-3-en-1-yl and 1-methylbut-2-en-1-yl; alkynyl is, for example, propargyl, but-2-yn-1-yl, but-3-yn-1-yl, 1-methylbut-3-yn-1-yl. The multiple bond may be in any position of the unsaturated radical.

Cycloalkyl is a carbocyclic saturated ring system having three to nine carbon atoms, for example cyclopropyl, cyclopentyl or cyclohexyl. In the case of composite radicals, such as (C₃-C₈)-cycloalkyl-(C₁-C₆)-alkyl, the first-mentioned radical may be in any position of the radical mentioned second.

In the case of a doubly substituted amino group, such as dialkylamino, these two substituents may be identical or different.

Halogen is fluorine, chlorine, bromine or iodine. Haloalkyl, -alkenyl and -alkynyl are alkyl, alkenyl or alkynyl which are partially or completely substituted by halogen, preferably by fluorine, chlorine and/or bromine, in particular by fluorine or chlorine, for example CF₃, CHF₂, CH₂F, CF₃CF₂, CH₂FCHCl, CCl₃, CHCl₂, CH₂CH₂Cl; haloalkoxy is, for example, OCF₃, OCHF₂, OCH₂F, CF₃CF₂O, OCH₂CF₃ and OCH₂CH₂Cl; this applies correspondingly to haloalkenyl and other halogen-substituted radicals.

An acyl radical is the radical of an organic acid, for example the radical of a carboxylic acid and radicals of acids derived therefrom, such as the thiocarboxylic acid, unsubstituted or N-substituted iminocarboxylic acids or the radical of carbonic monoesters, unsubstituted or N-substituted carbaminic acid, sulfonic acids, sulfinic acids, phosphonic acids, phosphinic acids. Acyl is, for example, formyl, alkylcarbonyl, such as (C₁-C₄)-alkylcarbonyl, phenylcarbonyl, where the phenyl ring is unsubstituted or substituted, for example as shown for phenyl above, or alkyloxycarbonyl, phenyloxycarbonyl, benzyloxycarbonyl, alkylsulfonyl, alkylsulfinyl, N-alkyl-1-iminoalkyl or other radicals of organic acids.

A hydrocarbon radical is a straight-chain, branched or cyclic and saturated or unsaturated aliphatic or aromatic hydrocarbon radical, for example alkyl, alkenyl, alkynyl, cycloalkyl, cycloalkenyl, phenyl, naphthyl, tetrahydronaphthyl, indenyl, indanyl, pentalenyl and fluorenyl.

Substituted radicals, such as substituted hydrocarbon radicals, for example substituted alkyl, alkenyl, alkynyl, aryl, phenyl and benzyl, or substituted heterocyclyl are, for example, a radical which is derived from the unsubstituted parent compound and which is substituted by one or more identical or different other radicals, the substituents being, for example, one or more, preferably, unless mentioned otherwise, 1, 2 or 3 radicals, from the group consisting of halogen, alkoxy, haloalkoxy, alkylthio, hydroxyl, amino, nitro, carboxyl, cyano, azido, alkoxycarbonyl, alkylcarbonyl, formyl, carbamoyl, mono- and dialkylaminocarbonyl, substituted amino, such as acylamino, mono- and dialkylamino, and alkylsulfinyl, haloalkylsulfinyl, alkylsulfonyl, haloalkylsulfonyl and, in the case of cyclic radicals, also alkyl and haloalkyl, and unsaturated aliphatic radicals which correspond to the abovementioned saturated hydrocarbon-containing radicals, such as alkenyl, alkynyl, alkenyloxy, alkynyloxy etc. Preferred radicals with carbon atoms are those having up to 4 carbon atoms. Preference is usually given to substituents from the group consisting of halogen, for example fluorine and chlorine, (C₁-C₄)-alkyl, preferably methyl or ethyl, (C₁-C₄)-haloalkyl, preferably trifluoromethyl, (C₁-C₄)-alkoxy, preferably methoxy or ethoxy, (C₁-C₄)-haloalkoxy, nitro and cyano. Particular preference is given here to the substituents methyl, methoxy and chlorine.

If the carbon chain of an alkyl, alkenyl or alkynyl radical is interrupted by more than one heteroatom, these heteroatoms are not to be directly adjacent.

Unless indicated otherwise, the term heterocycle means a saturated, unsaturated or heteroaromatic ring system which contains one or more heteroatoms, preferably from the group consisting of oxygen, sulfur and nitrogen. Examples include pyridine, pyrimidine, pyridazine, pyrazine, triazine, quinoline, isoquinoline, quinoxaline, quinazoline, cinnoline, thiophene, thiazole, oxazole, oxetan, furan, pyrrole, pyrazole, imidazole, aziridine, oxiran, pyrrolidine, piperidine, piperazine, dioxolan, morpholine, tetrahydrofuran. The terms “heterocyclyl” and “heterocyclic radical” are also to be understood in this sense, i.e. as radicals which are derived from the above-mentioned heterocycles. If a heterocycle contains at least two heteroatoms, the condition applies that two oxygen atoms may not be directly adjacent. These heterocycles may in principle, i.e. the structure of the ring system permitting, be attached with the other moiety of the molecule of the compound of the formula I at any position of the heterocycle. Unless indicated otherwise, the heterocycle may be unsubstituted or substituted by one or more identical or different radicals. These radicals may be the radicals mentioned under “substituted hydrocarbon radicals”, and additionally also oxo. The oxo group may also be present at the hetero ring atoms which can exist in different oxidation states, for example at nitrogen and sulfur.

A ring may be saturated, partially saturated, completely unsaturated or aromatic.

The abovementioned examples of radicals or ranges of radicals which are included in the general terms, such as “alkyl”, “acyl”, “substituted radicals”, etc., are not meant to be a complete list. The general terms also include the definitions of radical ranges in groups of preferred compounds listed further below, in particular radical ranges which include the specific radicals from the examples in the tables.

Depending on the type and the attachment of the substituents, the compounds of the formula I may be present as stereoisomers. If, for example, one or more alkenyl groups are present, this may lead to diastereomers. If, for example, one or more asymmetric carbon atoms are present, this may lead to enantiomers and diastereomers. Stereoisomers can be obtained from the mixtures resulting in the preparation by using customary separation methods, for example chromatographic separation processes. Stereoisomers can also be prepared selectively by employing stereoselective reactions using optically active starting materials and/or auxiliaries. The invention also relates to all stereoisomers and mixtures thereof which are embraced by the formula I but not defined specifically.

The compounds of the formula I also include tautomers of the compounds which are formed by hydrogen shifts, such as, for example, enols and others, even if they do not formally correspond to the formula I.

Depending on the type of the substituents, the compounds of the formula I can form salts and are embraced by the term “compounds (of the formula) (I)”. In substituents with acidic properties, such as COOH and SO₃H, the hydrogen atom of the acidic group may be replaced by an agriculturally suitable cation. These salts are, for example, metal salts, in particular alkali metal salts or alkaline earth metal salts, in particular sodium and potassium salts, or else ammonium salts or salts with organic amines. Salt formation may also occur by addition of an acid to basic substituents, such as, for example, amino and alkylamino. Acids which are suitable for this purpose are strong inorganic and organic acids, for example HCl, HBr, H₂SO₄ or HNO₃. Such salts also form part of the subject matter of the invention.

The possibilities of combining the various substituents of the formula I are to be understood such that the general principles of construction of chemical compounds have to be observed, i.e. that no compounds are formed which are known to the person skilled in the art to be chemically unstable or impossible.

Preference is given to compounds of the formula I and salts thereof in which

aryl is an unsubstituted or substituted mono- or bicyclic aromatic radical having 5 to 10 ring atoms, 1, 2, 3 or 4 of which may in each case independently of one another originate from the group consisting of oxygen, sulfur and nitrogen, and/or

—Y— is a divalent unit from the group consisting of —O—, —S—, —NR¹¹—, —NR¹²CONR¹³—, —CO₂—, —OCONR¹⁴—, —SO—, —SO₂—, —SO₂O—, —SO₂NR¹⁴—, —O—NR¹¹—, —NH—NH— and —O—CR^{a}R^{b}—CR^{c}R^{d}—O—, in which R^{a}, R^{b}, R^{c} and R^{d} independently of one another are each H, CH₃ or C₂H₅, or else a trivalent unit of the formula —O—N═ and/or

R¹, R² independently of one another are each a radical from a group G3 which comprises hydrogen, (C₁-C₈)-alkyl, (C₂-C₆)-alkenyl, (C₂-C₆)-alkynyl, (C₁-C₆)-alkoxy, (C₃-C₈)-cycloalkyl, (C₃-C₈)-cycloalkoxy, aryl-(C₁-C₆)-alkyl and (C₃-C₈)-cycloalkyl-(C₁-C₆)-alkyl, where the cyclic moiety of the four last-mentioned radicals is in each case unsubstituted or substituted by one or more identical or different radicals from the group consisting of halogen, nitro, cyano and —B—X¹, where —B— and X¹ are as defined below, and where the non-cyclic moiety of the eight last-mentioned radicals from the group G3 is in each case unsubstituted or substituted by one or more identical or different radicals from the group consisting of halogen, nitro, cyano and —B—X², where X² is as defined below, and where the non-cyclic moiety of the radicals from the group G3 may in each case be interrupted by one or more identical or different heteroatoms from the group consisting of oxygen and sulfur, or else

two R¹ of two directly adjacent (CR¹R²) groups together with the bond between the carbon atoms of the groups form a double bond, or two R¹ and two R² of two directly adjacent (CR¹R²) groups together with the bond between the carbon atoms of the groups form a triple bond or

R¹ is a linkage point for the double bond in the case where Y is a trivalent unit ═N—O— adjacent to a CR¹R² group, and/or

R³ and R⁴ independently of one another are each a radical from a group G4 which comprises hydrogen, (C₁-C₈)-alkyl, (C₂-C₆)-alkenyl, (C₂-C₆)-alkynyl, (C₁-C₆)-alkoxy, (C₃-C₈)-cycloalkyl, (C₃-C₈)-cycloalkoxy, aryl, aryl-(C₁-C₆)-alkyl, aryl-(C₁-C₆)-alkoxy, (C₃-C₈)-cycloalkyl-(C₁-C₆)-alkyl, (C₃-C₈)-cycloalkyl-(C₁-C₆)-alkoxy, (C₃-C₈)-cycloalkoxy-(C₁-C₆)-alkyl and (C₃-C₈)-cycloalkoxy-(C₁-C₆)-alkoxy, where the cyclic moiety of the nine last-mentioned radicals is in each case unsubstituted or substituted by one or more identical or different radicals from the group consisting of halogen, nitro, cyano, thiocyanato and —B—X¹, where —B— and X¹ are as defined below, and where the non-cyclic moiety of the thirteen last-mentioned radicals from the group G4 in each case is unsubstituted or substituted by one or more identical or different radicals from the group consisting of halogen, nitro, cyano, thiocyanato and —B—X², where X² is as defined below, and where the non-cyclic moiety of the radicals from the group G4 may in each case be interrupted by one or two identical or different heteroatoms from the group consisting of oxygen and sulfur, or else two R¹ of two directly adjacent (CR¹R²) groups together with the bond between the carbon atoms of the groups form a double bond or else

two R³ of two directly adjacent (CR³R⁴) groups together with the bond between the carbon atoms of the groups form a double bond, or two R³ and two R⁴ of two directly adjacent (CR³R⁴) groups together with the bond between the carbon atoms of the groups form a triple bond or else

R³ is a linkage point for the double bond in the case where Y is a trivalent unit ═N—O— adjacent to a CR³R⁴ group, and/or

—B— is a direct bond or a divalent unit from the group consisting of —O—, —S—, —NR¹¹—, —CO—, —CO₂—, —OCO₂—, —SO—, —SO₂—, —SO₂O— and —SO₂NR¹⁴— and/or

X¹ is hydrogen, (C₁-C₆)-alkyl, (C₂-C₆)-alkenyl, (C₂-C₆)-alkynyl, (C₃-C₆)-cycloalkyl or heterocyclyl having 3 to 6 ring atoms, 1, 2 or 3 of which originate from the group consisting of nitrogen, oxygen and sulfur, and where the five last-mentioned radicals are unsubstituted or substituted by one or more identical or different halogen atoms, and/or

X² is hydrogen or heterocyclyl having 3 to 9 ring atoms, 1, 2 or 3 of which originate from the group consisting of nitrogen, oxygen and sulfur, which is unsubstituted or substituted by 1, 2 or 3 identical or different halogen atoms, and/or

R⁵, R⁶ independently of one another are each a radical from the group G4 and/or

R⁷ is hydrogen, amino, alkylcarbonyl, alkylamino or dialkylamino having in each case one to four carbon atoms in the alkyl radical, is an acyclic or cyclic hydrocarbon radical having in each case one to six carbon atoms, or is heterocyclyl having three to six ring atoms and one to three hetero ring atoms from the group consisting of nitrogen, oxygen and sulfur, where each of the six last-mentioned radicals is unsubstituted or substituted by one or more identical or different radicals from the group consisting of halogen, (C₁-C₄)-alkoxy, halo-(C₁-C₄)-alkoxy, (C₁-C₄)-alkylthio, (C₂-C₄)-alkenyl, (C₂-C₄)-alkynyl, (C₂-C₄)-alkenyloxy, (C₂-C₄)-alkynyloxy, hydroxyl, amino, alkylamino, dialkylamino, nitro, carboxyl, cyano, (C₁-C₄)-alkoxycarbonyl, (C₁-C₄)-alkylcarbonyl, formyl, carbamoyl, mono- and di-(C₁-C₄)-alkylaminocarbonyl, (C₁-C₄)-alkylsulfonyl, halo-(C₁-C₄)-alkylsulfonyl and, in the case of cyclic radicals, also (C₁-C₄)-alkyl and halo-(C₁-C₄)-alkyl, and/or

R⁸ is (C₁-C₆)-alkyl, which is unsubstituted or substituted by one or more identical or different radicals from the group consisting of halogen, cyano, nitro, thiocyanato, hydroxyl, (C₁-C₄)-alkoxy, (C₁-C₄)-alkylthio, (C₁-C₄)-alkylsulfonyl, (C₂-C₄)-alkenyl, (C₂-C₄)-alkynyl, phenyl, (C₃-C₉)-cycloalkyl, (C₃-C₉)-cycloalkoxy and heterocyclyl having three to six ring atoms and one to three hetero ring atoms from the group consisting of oxygen, nitrogen and sulfur which is unsubstituted or substituted by one or more identical or different radicals from the group consisting of halogen, amino, (C₁-C₄)-alkyl, (C₁-C₄)-alkoxy, halo-(C₁-C₄)-alkyl and halo-(C₁-C₄)-alkoxy, is (C₃-C₈)-cycloalkyl or (C₃-C₈)-cycloalkoxy, where the two last-mentioned radicals are unsubstituted or substituted by one or more identical or different radicals from the group consisting of halogen, nitro, cyano, (C₁-C₄)-alkyl, (C₁-C₄)-alkoxy, halo-(C₁-C₄)-alkyl and halo-(C₁-C₄)-alkoxy, and/or

R⁹, R¹⁰ independently of one another are each hydrogen, amino, (C₁-C₆)-alkylcarbonyl, (C₁-C₆)-alkylamino, di-[(C₁-C₆)-alkyl]amino, (C₁-C₆)-alkyl, (C₃-C₈)-cycloalkyl, (C₁-C₆)-alkoxy, (C₃-C₈)-cycloalkoxy or heterocyclyl having 3 to 6 ring atoms and 1 to 3 hetero ring atoms from the group consisting of oxygen, nitrogen and sulfur, where each of the eight last-mentioned radicals is unsubstituted or substituted by one or more identical or different radicals from the group consisting of halogen, amino, hydroxyl and, in the case of cyclic radicals, also (C₁-C₄)-alkyl, (C₁-C₄)-alkoxy, halo-(C₁-C₄)-alkyl and halo-(C₁-C₄)-alkoxy, and/or

R¹¹ is hydrogen, amino, (C₁-C₆)-alkylamino, di-[(C₁-C₆)-alkyl]amino, (C₁-C₆)-alkyl, (C₃-C₈)-cycloalkyl, (C₃-C₈)-cycloalkyl-(C₁-C₆)-alkyl, (C₁-C₆)-alkoxy, (C₁-C₆)-alkoxy-(C₁-C₆)-alkoxy, (C₃-C₈)-cycloalkoxy, (C₁-C₆)-alkylcarbonyl, where the nine last-mentioned radicals are unsubstituted or substituted by one or more identical or different radicals from the group consisting of halogen, amino, hydroxyl and, in the case of cyclic radicals, also (C₁-C₄)-alkyl, (C₁-C₄)-alkoxy, halo-(C₁-C₄)-alkyl and halo-(C₁-C₄)-alkoxy, and/or

R¹², R¹³ independently of one another are each hydrogen, (C₁-C₆)-alkyl, (C₂-C₆)-alkenyl, (C₂-C₆)-alkynyl, phenyl, phenyl-(C₁-C₆)-alkyl, (C₃-C₈)-cycloalkyl, (C₃-C₈)-cycloalkyl-(C₁-C₆)-alkyl, where the cyclic moiety of the four last-mentioned radicals is in each case unsubstituted or substituted by one or more identical or different radicals from the group consisting of (C₁-C₄)-alkyl, halo-(C₁-C₄)-alkyl, (C₁-C₄)-alkoxy and halo-(C₁-C₄)-alkoxy, or

R¹² and R¹³ together with the N—CO—N group which carries them form a 5- to 8-membered ring which, in addition to the two abovementioned nitrogen atoms, may contain a further heteroatom from the group consisting of oxygen, nitrogen and sulfur and which is unsubstituted or substituted by one or more identical or different radicals from the group consisting of halogen, (C₁-C₄)-alkyl and halo-(C₁-C₄)-alkyl;

R¹⁴ is hydrogen or (C₁-C₆)-alkyl or (C₃-C₈)-cycloalkyl, each of which is unsubstituted or substituted by one or more identical or different radicals from the group consisting of halogen, phenyl, hydroxyl, (C₁-C₄)-alkoxy and halo-(C₁-C₄)-alkoxy and/or

R¹⁵ and R¹⁶ independently of one another are each hydrogen, phenyl, (C₁-C₆)-alkoxy, phenyl-(C₁-C₆)-alkyl, (C₁-C₆)-alkyl, (C₁-C₆)-alkylthio, where the five last-mentioned radicals are unsubstituted or substituted, and where the aliphatic carbon skeleton of the three last-mentioned radicals may be interrupted by one or more identical or different heteroatoms from the group consisting of oxygen and sulfur.

Preference is also given to compounds of the formula I and salts thereof in which

R¹ and R² of a (CR¹R²) group together with the carbon atom which carries them form a carbonyl group, a group CR¹⁵R¹⁶ or 3- to 6-membered ring which may contain one or two identical or different heteroatoms from the group consisting of oxygen, nitrogen and sulfur and which is unsubstituted or substituted by one or more identical or different radicals from the group consisting of halogen, nitro, cyano and —B—X¹, or

two R¹ of two directly adjacent or not directly adjacent (CR¹R²) groups together with the carbon atoms which carry or link them form an unsubstituted or substituted 3- to 6-membered ring which may contain one or two identical or different heteroatoms from the group consisting of oxygen, nitrogen and sulfur and which is unsubstituted or substituted by one or more identical or different radicals from the group consisting of halogen, nitro, cyano and —B—X¹.

Likewise, preference is given to compounds of the formula I in which

R³ and R⁴ together with the carbon atom which carries them form a carbonyl group, a group CR¹⁵R¹⁶ or a 3- to 6-membered ring, which may contain one or two identical or different heteroatoms from the group consisting of oxygen, nitrogen and sulfur and which is unsubstituted or substituted by one or more identical or different radicals from the group consisting of halogen, nitro, cyano and —B—X¹, or

two R³ of two directly adjacent or not directly adjacent (CR³R⁴) groups together with the carbon atoms which carry or link them form an unsubstituted or substituted 3- to 6-membered ring which may contain one or two identical or different heteroatoms from the group consisting of oxygen, nitrogen and sulfur and which is unsubstituted or substituted by one or more identical or different radicals from the group consisting of halogen, nitro, cyano and —B—X¹.

Moreover, preference is given to compounds of the formula I and salts thereof in which

R³ and R⁵ of two directly adjacent or not directly adjacent (CR³R⁴)— or (CR⁵R⁶)— groups together with the carbon atoms which link them form an unsubstituted or substituted 3- to 6-membered ring which may contain one or two identical or different heteroatoms from the group consisting of oxygen, nitrogen and sulfur and which is unsubstituted or substituted by one or more identical or different radicals from the group consisting of halogen, nitro, cyano and —B—X¹, or

R⁵ and R⁶ together with the carbon atom which carries them form a carbonyl group, a group CR¹⁵R¹⁶ or a 3- to 6-membered ring which may contain one or two identical or different heteroatoms from the group consisting of oxygen, nitrogen and sulfur and which is unsubstituted or substituted by one or more identical or different radicals from the group consisting of halogen, nitro, cyano and —B—X¹, or

R⁶ is heterocyclyl.

Likewise, preference is given to compounds of the formula I and salts thereof in which

R⁹ and R¹⁰ together with the nitrogen atom which carries them form a heterocycle having a total of three to six ring atoms and from among these one to four hetero ring atoms, where the other hetero ring atoms which may be present in addition to the nitrogen atom that is present are selected from the group consisting of oxygen, nitrogen and sulfur and where this heterocycle is unsubstituted or substituted by up to three identical or different radicals preferably from the group consisting of halogen, amino, hydroxyl, (C₁-C₄)-alkyl, (C₁-C₄)-alkoxy, halo-(C₁-C₄)-alkyl and halo-(C₁-C₄)-alkoxy.

Moreover, preference is given to compounds of the formula I and salts thereof in which

R¹⁵ and R¹⁶ together with the carbon atom which carries them form a 3- to 6-membered ring which may contain one or two identical or different heteroatoms from the group consisting of oxygen, nitrogen and sulfur and which is unsubstituted or substituted by one or more identical or different radicals from the group consisting of halogen, (C₁-C₄)-alkyl and halo-(C₁-C₄)-alkyl.

Particular preference is given to compounds of the formula I and salts thereof in which

aryl is a mono- or bicyclic aromatic radical having 5 to 10 ring atoms, 1, 2, 3 or 4 of which may in each case independently of one another originate from the group consisting of oxygen, sulfur and nitrogen, which is unsubstituted or substituted by one or more identical or different radicals from the group consisting of halogen, hydroxyl, amino, formyl, (C₁-C₄)-alkoxycarbonyl, (C₁-C₄)-alkylcarbonyl, halo-(C₁-C₄)-alkoxycarbonyl, halo-(C₁-C₄)-alkylcarbonyl, (C₁-C₄)-alkyl, (C₂-C₄)-alkenyl, (C₂-C₄)-alkynyl, (C₁-C₄)-alkoxy, (C₁-C₄)-alkoxy-(C₁-C₄)-alkyl, halo-(C₁-C₄)-alkoxy-(C₁-C₄)-alkyl, halo-(C₁-C₄)-alkoxy-[halo-(C₁-C₄)-alkyl], (C₁-C₄)-alkoxy-[halo-(C₁-C₄)-alkyl], (C₃-C₆)-cycloalkyl, (C₃-C₆)-Cycloalkyl-(C₁-C₄)-alkyl, (C₃-C₆)-cycloalkyl-[halo-(C₁-C₄)-alkyl], halo-(C₃-C₆)-cycloalkyl-[halo-(C₁-C₄)-alkyl], halo-(C₃-C₆)-cycloalkyl-(C₁-C₄)-alkyl, (C₁-C₆)-alkylamino, di-[(C₁-C₆)-alkyl]amino, (C₁-C₆)-alkylaminocarbonyl, aminosulfonyl, (C₁-C₆)-alkylaminosulfonyl, di-[(C₁-C₆)-alkyl]aminosulfonyl, halo-(C₁-C₄)-alkyl and halo-(C₁-C₄)-alkoxy,

—Y— is a divalent unit from the group consisting of —O—, —S—, —NR¹¹—, —CO₂—, —SO—, —SO₂—, —SO₂O— and —SO₂NR¹⁴— or a unit from the group consisting of —NH—CO—NH— and —OCONH— or a unit from the group consisting of —O—NH—, —O—N═ and —OCH₂CH₂O—,

R¹, R² independently of one another are in each case a radical from a group G5 which comprises hydrogen, (C₁-C₆)-alkyl, (C₂-C₄)-alkenyl, (C₂-C₄)-alkynyl, (C₁-C₆)-alkoxy, (C₃-C₆)-cycloalkyl, (C₃-C₆)-cycloalkoxy, aryl-(C₁-C₆)-alkyl and (C₃-C₆)-cycloalkyl-(C₁-C₆)-alkyl, where the cyclic moiety of the four last-mentioned radicals is in each case unsubstituted or substituted by one or more identical or different radicals from the group consisting of halogen, nitro, cyano and —B—X¹, where —B— and X¹ are as defined below, and where the non-cyclic moiety of the eight last-mentioned radicals from the group G5 is in each case unsubstituted or substituted by one or more identical or different radicals from the group consisting of halogen, nitro, cyano and —B—X², where X² is as defined below, and where the non-cyclic moiety of the radicals from the group G5 may in each case be interrupted by one or more identical or different heteroatoms from the group consisting of oxygen and sulfur, or

two R¹ of two directly adjacent (CR¹R²) groups together with the bond between the carbon atoms of the groups form a double bond, or two R¹ and two R² of two directly adjacent (CR¹R²) groups together with the bond between the carbon atoms of the groups form a triple bond or

R¹ is a linkage point for the double bond in the case where Y is a trivalent unit ═N—O— adjacent to a CR¹R² group,

R³, R⁴ independently of one another are in each case a radical from a group G6 which comprises hydrogen, (C₁-C₆)-alkyl, (C₂-C₄)-alkenyl, (C₂-C₄)-alkynyl, (C₁-C₆)-alkoxy, (C₃-C₈)-cycloalkyl, (C₃-C₈)-cycloalkoxy, aryl, aryl-(C₁-C₄)-alkyl, aryl-(C₁-C₄)-alkoxy, (C₃-C₈)-cycloalkyl-(C₁-C₄)-alkyl, (C₃-C₈)-cycloalkyl-(C₁-C₄)-alkoxy, (C₃-C₈)-cycloalkoxy-(C₁-C₄)-alkyl and (C₃-C₈)-cycloalkoxy-(C₁-C₄)-alkoxy, where the cyclic moiety of the nine last-mentioned radicals is in each case unsubstituted or substituted by one or more identical or different radicals from the group consisting of halogen, nitro, cyano and —B—X¹, where —B— and X¹ are as defined below, and where the non-cyclic moiety of the thirteen last-mentioned radicals from the group G6 is in each case unsubstituted or substituted by one or more identical or different radicals from the group consisting of halogen, nitro, cyano and —B—X², where X² is as defined below, and where the non-cyclic moiety of the radicals from the group G6 may in each case be interrupted by a heteroatom from the group consisting of oxygen and sulfur, or else

two R³ of two directly adjacent (CR³R⁴) groups together with the bond between the carbon atoms of the groups form a double bond, or two R³ and two R⁴ of two directly adjacent (CR³R⁴) groups together with the bond between the carbon atoms of the groups form a triple bond or else

R³ is a linkage point for the double bond in the case where Y is a trivalent unit —O—N═ adjacent to a CR³R⁴ group, and

—B— is a direct bond or a divalent unit from the group consisting of —O—, —S—, —NR¹¹—, —CO—, —CO₂—, —SO—, —SO₂— and —SO₂NR¹⁴—,

X¹ is hydrogen, (C₁-C₆)-alkyl, (C₂-C₄)-alkenyl, (C₂-C₄)-alkynyl, (C₃-C₆)-cycloalkyl or heterocyclyl having 3 to 6 ring atoms, 1, 2 or 3 of which originate from the group consisting of nitrogen, oxygen and sulfur, and where the five last-mentioned radicals are unsubstituted or substituted by up to five identical or different halogen atoms,

X² is hydrogen or is heterocyclyl having 3 to 6 ring atoms, 1, 2 or 3 of which originate from the group consisting of nitrogen, oxygen and sulfur, which is unsubstituted or substituted by 1, 2 or 3 identical or different halogen atoms,

R⁵, R⁶ independently of one another are each a radical from the group G6,

R⁷ is hydrogen, amino, alkylcarbonyl, alkylamino or dialkylamino having in each case one to four carbon atoms in the alkyl radical, or is an acyclic or cyclic hydrocarbon radical having in each case one to six carbon atoms, where each of the five last-mentioned radicals is unsubstituted or substituted by one or more identical or different radicals from the group consisting of halogen, (C₁-C₄)-alkoxy, halo-(C₁-C₄)-alkoxy, (C₂-C₄)-alkenyl, (C₂-C₄)-alkynyl, (C₂-C₄)-alkenyloxy, (C₂-C₄)-alkynyloxy, hydroxyl, amino, (C₁-C₄)-alkylamino, di-[(C₁-C₄)-alkyl]amino, nitro, carboxyl, cyano, (C₁-C₄)-alkoxycarbonyl, (C₁-C₄)-alkylcarbonyl, formyl, carbamoyl, (C₁-C₄)-alkylsulfonyl, halo-(C₁-C₄)-alkylsulfonyl and, in the case of cyclic radicals, also (C₁-C₄)-alkyl and halo-(C₁-C₄)-alkyl,

R⁸ is (C₁-C₆)-alkyl which is unsubstituted or substituted by one or more identical or different radicals from the group consisting of halogen, cyano, hydroxyl, (C₁-C₄)-alkoxy, (C₁-C₄)-alkylthio, (C₁-C₄)-alkylsulfonyl, (C₂-C₄)-alkenyl, (C₂-C₄)-alkynyl, phenyl, (C₃-C₉)-cycloalkyl, (C₃-C₉)-cycloalkoxy, is (C₃-C₈)-cycloalkyl or (C₃-C₈)-cycloalkoxy, where the two last-mentioned radicals are substituted or substituted by one or more identical or different radicals from the group consisting of halogen, nitro, cyano, (C₁-C₄)-alkyl, (C₁-C₄)-alkoxy, halo-(C₁-C₄)-alkyl and halo-(C₁-C₄)-alkoxy,

R⁹, R¹⁰ independently of one another are each hydrogen, amino, (C₁-C₄)-alkylcarbonyl, (C₁-C₄)-alkylamino, di-[(C₁-C₄)-alkyl]amino, (C₁-C₄)-alkyl, (C₃-C₈)-cycloalkyl, (C₁-C₄)-alkoxy, (C₃-C₈)-cycloalkoxy or heterocyclyl having 3 to 6 ring atoms and 1 to 3 hetero ring atoms from the group consisting of oxygen, nitrogen and sulfur, where each of the eight last-mentioned radicals is unsubstituted or substituted by up to four identical or different radicals from the group consisting of halogen, amino, hydroxyl and, in the case of cyclic radicals, also (C₁-C₄)-alkyl, (C₁-C₄)-alkoxy, halo-(C₁-C₄)-alkyl and halo-(C₁-C₄)-alkoxy,

R¹¹ is hydrogen, amino, (C₁-C₄)-alkylamino, di-[(C₁-C₄)-alkyl]amino, (C₁-C₄)-alkyl, (C₃-C₈)-cycloalkyl, (C₃-C₈)-cycloalkyl-(C₁-C₄)-alkyl, (C₁-C₄)-alkoxy, (C₁-C₄)-alkoxy-(C₁-C₄)-alkoxy, (C₃-C₈)-cycloalkoxy, (C₁-C₄)-alkylcarbonyl, where the nine last-mentioned radicals are unsubstituted or substituted by one or more identical or different halogen atoms and, in the case of cyclic radicals, also (C₁-C₄)-alkyl, (C₁-C₄)-alkoxy, halo-(C₁-C₄)-alkyl and halo-(C₁-C₄)-alkoxy,

R¹⁴ is hydrogen or (C₁-C₄)-alkyl or (C₃-C₆)-cycloalkyl, each of which is unsubstituted or substituted by one or more identical or different radicals from the group consisting of halogen, (C₁-C₄)-alkoxy and halo-(C₁-C₄)-alkoxy and/or

R¹⁵, R¹⁶ independently of one another are each hydrogen, phenyl, (C₁-C₄)-alkoxy, phenyl-(C₁-C₄)-alkyl, (C₁-C₄)-alkyl, (C₁-C₄)-alkylthio, where the five last-mentioned radicals are unsubstituted or substituted by one or more identical or different halogen atoms and, in the case of cyclic radicals, also by one or more identical or different radicals from the group consisting of halogen, (C₁-C₄)-alkyl and halo-(C₁-C₄)-alkyl.

Preference is also given to compounds according to the invention of the formula I which only contain one or more of the abovementioned preferred radicals.

Particular preference is also given to compounds of the formula I, in which

R¹ and R² of a (CR¹R²) group together with the carbon atom which carries them form a carbonyl group, a group CR¹⁵R¹⁶ or a 3- to 6-membered ring which may contain one or two identical or different heteroatoms from the group consisting of oxygen, nitrogen and sulfur and which is unsubstituted or substituted by up to four identical or different radicals from the group consisting of halogen, nitro, cyano and —B—X¹, or

two R¹ of two directly adjacent or not directly adjacent (CR¹R²) groups together with the carbon atoms which carry or link them form an unsubstituted or substituted 3- to 6-membered ring which may contain one or two identical or different heteroatoms from the group consisting of oxygen, nitrogen and sulfur and which is unsubstituted or substituted by up to four identical or different radicals from the group consisting of halogen, nitro, cyano and —B—X¹.

Particular preference is likewise given to compounds of the formula I in which

R³ and R⁴ together with the carbon atom which carries them form a carbonyl group, a group CR¹⁵R¹⁶ or a 3- to 6-membered ring, which may contain one or two identical or different heteroatoms from the group consisting of oxygen, nitrogen and sulfur and which is unsubstituted or substituted by up to four identical or different radicals from the group consisting of halogen, nitro, cyano and —B—X¹, or

two R³ of two directly adjacent or not directly adjacent (CR³R⁴) groups together with the carbon atoms which carry or link them form an unsubstituted or substituted 3- to 6-membered ring which may contain one or two identical or different heteroatoms from the group consisting of oxygen, nitrogen and sulfur and which is unsubstituted or substituted by up to four identical or different radicals from the group consisting of halogen, nitro, cyano and —B—X¹.

Moreover, particular preference is given to compounds of the formula I in which

R³ and R⁵ of two directly adjacent or not directly adjacent (CR³R⁴)— or (CR⁵R⁶)— groups together with the carbon atoms which link them form an unsubstituted or substituted 3- to 6-membered ring which may contain one or two identical or different heteroatoms from the group consisting of oxygen, nitrogen and sulfur and which is unsubstituted or substituted by up to four identical or different radicals from the group consisting of halogen, nitro, cyano and —B—X¹, or

R⁵ and R⁶ together with the carbon atom which carries them form a carbonyl group, a group CR¹⁵R¹⁶ or a 3- to 6-membered ring which may contain one or two identical or different heteroatoms from the group consisting of oxygen, nitrogen and sulfur and which is unsubstituted or substituted by one or more identical or different radicals from the group consisting of halogen, nitro, cyano, thiocyanato and —B—X¹, or

R⁶ is heterocycyl.

Particular preference is also given to compounds of the formula I in which

R⁹ and R¹⁰ together with the nitrogen atom which carries them form a heterocycle having a total of three to six ring atoms and from among these one to four hetero ring atoms, where the other hetero ring atoms which may be present in addition to the nitrogen atom that is present are selected from the group consisting of oxygen, nitrogen and sulfur and where this heterocycle is unsubstituted or substituted.

Particular preference is likewise given to compounds of the formula I in which

R¹⁵ and R¹⁶ together with the carbon atom which carries them form a 3-6-membered ring which may contain one or two identical or different heteroatoms from the group consisting of oxygen, nitrogen and sulfur and which is unsubstituted or substituted by up to four identical or different radicals from the group consisting of halogen, (C₁-C₄)-alkyl and halo-(C₁-C₄)-alkyl.

Very particular preference is given to compounds of the formula I in which

aryl is phenyl, naphthyl, pyridinyl, pyrimidinyl, furanyl, benzofuranyl, pyrrolyl, oxazolyl, thiazolyl, imidazolyl, oxadiazolyl or thienyl, each of which is unsubstituted or substituted by one or more identical or different radicals from the group consisting of halogen, hydroxyl, amino, formyl, (C₁-C₄)-alkoxycarbonyl, (C₁-C₄)-alkylcarbonyl, (C₁-C₄)-alkyl, (C₂-C₄)-alkenyl, (C₂-C₄)-alkynyl, (C₁-C₄)-alkoxy, (C₁-C₄)-alkylamino, di-[(C₁-C₄)-alkyl]amino, aminocarbonyl, (C₁-C₄)-alkylaminocarbonyl, di-(C₁-C₄)-alkylaminocarbonyl, aminosulfonyl, (C₁-C₄)-alkylaminosulfonyl, di-[(C₁-C₄)-alkyl]aminosulfonyl, halo-(C₁-C₄)-alkyl, halo-(C₁-C₄)-alkoxy, (C₁-C₂)-alkylthio, (C₁-C₂)-alkylsulfinyl, (C₁-C₂)-alkylsulfonyl, halo-(C₁-C₂)-alkylthio, halo-(C₁-C₂)-alkylsulfinyl and halo-(C₁-C₂)-alkylsulfonyl;

—Y— is a divalent unit from the group consisting of —O—, —S—, —NR¹¹—, —CO₂—, —SO— and —SO₂— or else a divalent unit from the group consisting of —NH—CO—NH— and —O—CO—NH-or else a unit from the group consisting of —O—NH—, —O—N═ and —OCH₂CH₂O—,

m is an integer from 0 to 5, preferably 0, 1, 2 or 3, in particular O or 1;

n is an integer from 1 to 5, preferably 1, 2, 3 or 4, in particular 1, 2 or 3;

R¹, R² independently of one another are each a radical from a group G7 which comprises (C₁-C₄)-alkyl, (C₂-C₄)-alkenyl, (C₂-C₄)-alkynyl, (C₁-C₄)-alkoxy, hydrogen, (C₃-C₆)-Cycloalkyl, where the last-mentioned radical is unsubstituted or substituted by up to four identical or different radicals from the group consisting of halogen and —B—X¹, and where the four first-mentioned radicals from the group G7 are unsubstituted or substituted by one or more identical or different radicals from the group consisting of halogen and —B—X², where X² is as defined below, or else

R¹ and R² together with the carbon atom which carries them form a carbonyl group, or else

R¹ is a linkage point for the double bond in the case where Y is a trivalent unit ═N—O— which is adjacent to a CR¹R² group, and

R³, R⁴ independently of one another are each a radical from a group G8 which comprises hydrogen, (C₁-C₄)-alkyl, (C₂-C₄)-alkenyl, (C₂-C₄)-alkynyl, (C₁-C₆)-alkoxy, (C₃-C₆)-cycloalkyl, (C₃-C₆)-cycloalkoxy, aryl, aryl-(C₁-C₂)-alkyl, aryl-(C₁-C₂)-alkoxy, (C₃-C₆)-cycloalkyl-(C₁-C₂)-alkyl and (C₃-C₈)-cycloalkoxy-(C₁-C₄)-alkyl, where the cyclic moiety of the seven last-mentioned radicals is in each case unsubstituted or substituted by up to four identical or different radicals from the group consisting of halogen, nitro, cyano and —B—X¹, where —B— and X¹ are as defined below, and where the non-cyclic moiety of the eleven last-mentioned radicals from the group G8 is in each case unsubstituted or substituted by one or more identical or different radicals from the group consisting of halogen, nitro, cyano and —B—X², where X² is as defined below, or else

R³ and R⁴ together with the carbon atom which carries them form a carbonyl group, or else

R³ is a linkage point for the double bond in the case where Y is a trivalent unit —O—N═ which is adjacent to a CR³R⁴ group,

—B— is a direct bond or a divalent unit from the group consisting of —O—, —S—, —NR¹¹—, —CO— and —CO₂—;

X¹ is hydrogen, (C₁-C₄)-alkyl, (C₂-C₄)-alkenyl, (C₂-C₄)-alkynyl or (C₃-C₆)-cycloalkyl, the four last-mentioned radicals of which are unsubstituted or substituted by up to five identical or different halogen atoms,

X² is hydrogen or heterocyclyl having 3 to 6 ring atoms, 1, 2 or 3 of which originate from the group consisting of nitrogen, oxygen or sulfur, which is unsubstituted or substituted by one or more identical or different halogen atoms,

R⁵ is hydrogen, (C₁-C₄)-alkyl, halo-(C₁-C₄)-alkyl or (C₃-C₆)-cycloalkyl,

R⁶ is hydrogen, (C₁-C₄)-alkyl, (C₂-C₄)-alkenyl, (C₂-C₄)-alkynyl, (C₁-C₄)-alkoxy, (C₃-C₆)-cycloalkyl, (C₃-C₆)-cycloalkyl-(C₁-C₄)-alkyl, (C₃-C₆)-cycloalkoxy-(C₁-C₄)-alkyl, (C₃-C₆)-cycloalkoxy, (C₁-C₄)-alkoxy-(C₁-C₄)-alkyl or halo-(C₁-C₄)-alkyl,

R⁷ is hydrogen, amino or (C₁-C₄)-alkyl,

R⁸ (C₁-C₄)-alkyl, halo-(C₁-C₄)-alkyl, (C₃-C₆)-cycloalkyl, halo-(C₃-C₆)-cycloalkyl or (C₃-C₆)-cycloalkyl-(C₁-C₄)-alkyl,

R⁹, R¹⁰ independently of one another are each hydrogen, amino, formyl or (C₁-C₄)-alkyl, or

R¹¹ is hydrogen, amino, (C₁-C₄)-alkylamino, di-[(C₁-C₄)-alkyl]amino, (C₁-C₄)-alkyl or (C₃-C₆)-cycloalkyl, where the five last-mentioned radicals are unsubstituted or substituted by one or more identical or different halogen atoms, and/or

R¹⁴ is hydrogen or (C₁-C₄)-alkyl.

Particular preference is also given to compounds according to the invention of the formula I which contain only one or more of the abovementioned preferred radicals.

Very particular preference is also to given to compounds of the formula I in which two directly adjacent or not directly adjacent (CR¹R²) groups together with the carbon atoms which carry or link them form a 3-, 5- or 6-membered ring which may contain up to 2 heteroatoms from the group consisting of oxygen, sulfur and nitrogen and which is unsubstituted or substituted by up to four identical or different radicals from the group consisting of halogen and —B—X¹.

Very particular preference is likewise given to compounds of the formula I in which two R³ of two directly adjacent or not directly adjacent (CR³R⁴) groups together with the carbon atoms which carry or link them form a 3-, 5- or 6-membered ring which may contain up to two heteroatoms from the group consisting of oxygen, sulfur and nitrogen and which is unsubstituted or substituted by up to four identical or different radicals from the group consisting of halogen and —B—X¹.

Very particular preference is likewise given to compounds of the formula I in which

R³ and R⁵ of two directly adjacent or not directly adjacent (CR³R⁴)— or (CR⁵R⁶) groups together with the carbon atoms which link them form a 3-, 5- or 6-membered ring which may contain up to two heteroatoms from the group consisting of oxygen, sulfur and nitrogen and which is unsubstituted or substituted by up to four identical or different radicals from the group consisting of halogen and —B—X¹.

Moreover, very particular preference is given to compounds of the formula I in which

R⁹ and R¹⁰ together with the carbon atom which carries them form a heterocycle having three to six ring atoms and one to two hetero ring atoms, where the other hetero ring atoms which may be present in addition to the nitrogen atom which is present are selected from the group consisting of oxygen and nitrogen.

The present invention also provides processes for preparing the compounds of the formula I or salts thereof, which comprise

a) reacting a compound of the formula (II)

R⁸-Fu (II)

in which Fu is a functional group from the group consisting of carboxylic ester, carboxylic orthoester, carbonyl chloride, carboxamide, carboxylic anhydride and trichloromethyl

with a biguanidide of the formula (III) or an acid addition salt thereof

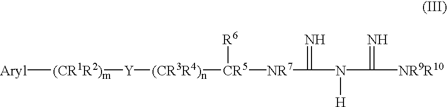

or

b) reacting a compounds of the formula (IV)

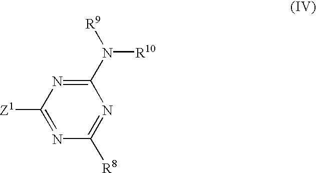

in which Z¹ is an exchangeable radical or a leaving group, for example chlorine, trichloromethyl, (C₁-C₄)-alkylsulfonyl or unsubstituted or substituted phenyl-(C₁-C₄)-alkylsulfonyl or (C₁-C₄)-alkylphenylsulfonyl, with a suitable amine of the formula (V) or an acid addition salt thereof

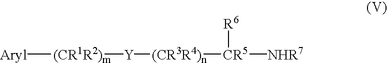

where in the formulae (II), (III), (IV) and (V) the radicals R¹ to R¹⁰, Y and n and m are as defined under formula (I).

The compounds of the formulae (II) and (III) are preferably reacted base-catalyzed in an inert organic solvent, such as, for example, tetrahydrofuran, (THF), dioxane, acetonitrile, dimethylformamide (DMF), methanol and ethanol, at temperatures between −10° C. and the boiling point of the solvent, preferably between 20° C. and 60° C. If acid addition salts of the formula (III) are used, these are usually liberated in situ with the aid of a base. Suitable bases or basic catalysts are alkali metal hydroxides, alkali metal hydrides, alkali metal carbonates, alkali metal alkoxides, alkaline earth metal hydroxides, alkaline earth metal hydrides, alkaline earth metal carbonates or organic bases, such as triethylamine or 1,8-diazabicyclo[5.4.0]undec-7-ene (DBU). The base in question is employed, for example, in the range of from 0.1 to 3 molar equivalents, based on the compound of the formula (III). The compound of the formula (II) can be employed, in relation to the compound of the formula (III) for example, in equimolar amounts or in an excess of up to 2 molar equivalents. In principle, the corresponding processes are known from the literature (compare: Comprehensive Heterocyclic Chemistry, A. R. Katritzky, C. W. Rees, Pergamon Press, Oxford, N.Y., 1984, Vol.3; Part 2B; ISBN 0-08-030703-5, p.290).

The compounds of the formulae (IV) and (V) are preferably reacted base-catalyzed in an inert organic solvent, such as, for examle, THF, dioxane, acetonitrile, DMF, methanol and ethanol, at temperatures between −10° C. and the boiling point of the solvent or solvent mixture in question, preferably at from 20° C. to 60° C.; the compound (V), if it is employed as an acid addition salt, is liberated in situ with a base, if appropriate. Suitable bases or basic catalysts are alkali metal hydroxides, alkali metal hydrides, alkali metal carbonates, alkali metal alkoxides, alkaline earth metal hydroxides, alkaline earth metal hydrides, alkaline earth metal carbonates or organic bases, such as triethylamine or 1,8-diazabicyclo[5.4.0]undec-7-ene (DBU). The base in question is usually employed in the range from 1 to 3 molar equivalents, based on the compound of the formula (IV), and the compound of the formula (IV) can be employed, for example, in equimolar amounts based on the compound of the formula (V), or in an excess of up to 2 molar equivalents. In principle, the corresponding processes are known from the literature (cf. Comprehensive Heterocyclic Chemistry, A. R. Katritzky, C. W. Rees, Pergamon Press, Oxford, N.Y., 1984, Vol.3, Part 2B, p. 482).

The starting materials of the formulae (II), (III), (IV) and (V) are either commercially available, or they can be prepared by or analogously to processes known from the literature. The compounds can also be prepared, for example, by one of the processes described below.

The compound of the formula (IV), or a direct presursor thereof, can be prepared, for example, as follows:

1. Reaction of a compound of the formula (II) with an amidinothiourea derivative of the formula (VI),

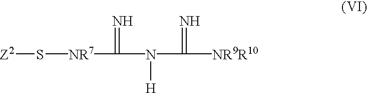

in which Z² is (C₁-C₄)-alkyl or phenyl-(C₁-C₄)-alkyl and R⁹ and R¹⁰ are as defined under formula (I) gives compounds of the formula (IV) in which Z¹=—SZ².

2. Reaction of a n amidine of the formula (VII) or an acid addition salt thereof,

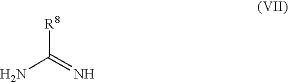

in which R⁸ is as defined under formula (I)

with an N-cyano dithioiminocarbonate of the formula (VIII),

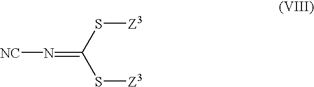

in which Z³ is (C₁-C₄)-alkyl or phenyl-(C₁-C₄)-alkyl gives compounds of the formula (IV) in which Z¹=—S-Z³.

3. Reaction of an alkali metal dicyanamide with a carboxylic acid derivative of the abovementioned formula (II) gives compounds of the formula (IV) in which Z¹=NH₂.

4. Reaction of trichloroacetonitrile with a nitrile of the formula (IX),

R⁸—CN (IX)

in which R⁸ is as defined under formula (I) initially gives compounds of the formula (X),

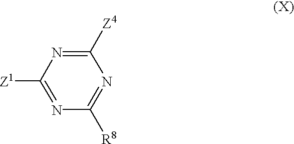

in which Z¹ and Z⁴ are each CCl₃ which, by subsequent reaction with compounds of the formula HNR⁹R¹⁰ (R⁹ and R¹⁰ as under formula (I)) give compounds of the formula (IV), in which Z¹=CCl₃.

The reaction of the carboxylic acid derivatives of the formula (II) with the amidinothiourea derivatives of the formula (VI) is preferably carried out base-catalyzed in an organic solvent, such as, for example, acetone, THF, dioxane, acetonitrile, DMF, methanol, ethanol, at temperatures from −10° C. to the boiling point of the solvent, preferably at from 0° C. to 20° C. However, the reaction can also be carried out in water or in aqueous solvent mixtures comprising one or more of the abovementioned organic solvents. If (VI) is employed as acid addition salt, it can be liberated in situ using a base, if appropriate. Suitable bases or basic catalysts are alkali metal hydroxides, alkali metal hydrides, alkali metal carbonates, alkali metal alkoxides, alkaline earth metal hydroxides, alkaline earth metal hydrides, alkaline earth metal carbonates or organic bases, such as triethylamine or 1,8-diazabicyclo[5.4.0]undec-7-ene (DBU). The base in question is employed, for example, in the range of from 1 to 3 molar equivalents, based on the compound of the formula (VI). Compounds of the formulae (II) and (VI) can be employed, for example, in equimolar amounts, or with an excess of up to 2 molar equivalents of the compound of the formula (II). In principle, the corresponding processes are known from the literature (cf.: H. Eilingsfeld, H. Scheuermann, Chem. Ber.; 1967, 100, 1874), the corresponding intermediates of the formula (IV) are novel.

The reaction of the amidines of the formula (VII) with the N-cyano dithioiminocarbonates of the formula (VIII) is preferably carried out base-catalyzed in an inert organic solvent, such as, for example, acetonitrile, DMF, dimethylacetamide (DMA), N-methylpyrrolidone (NMP), methanol and ethanol, at temperatures from −10° C. to the boiling point of the solvent, preferably at from 20° C. to 80° C. If (VII) is employed as acid addition salt, it can be liberated in situ using a base, if appropriate. Suitable bases or basic catalysts are alkali metal hydroxides, alkali metal hydrides, alkali metal carbonates, alkali metal alkoxides, alkaline earth metal hydroxides, alkaline earth metal hydrides, alkaline earth metal carbonates or organic bases, such as triethylamine or 1,8-diazabicyclo[5.4.0]undec-7-ene (DBU). The base in question is employed, for example, in the range of from 1 to 3 molar equivalents, based on the compound of the formula (VIII), compounds of the formulae (VII) and (VIII) can usually be employed in equimolar amounts or in an excess of 2 molar equivalents of the compound of the formula (II). In principle, the corresponding processes are known from the literature (cf.: T. A. Riley, W. J. Henney, N. K. Dailey, B. E. Wilson, R. K Robins; J. Heterocyclic Chem.; 1986, 23 (6), 1706-1714), the corresponding intermediates of the formula (IV) are novel.

Intermediates of the formula (X) where Z¹=chlorine can be prepared by reacting alkali metal dicyanamide with a carboxylic acid derivative of the formula (II), in which case Fu is preferably the functional group carbonyl chloride or carboxamide. The reaction components are, for example, reacted with acid catalysis in an inert organic solvent, such as, for example, toluene, chlorobenzene, chlorinated hydrocarbons, at temperatures between −10° C. and the boiling point of the solvent, preferably at from 20° C. to 80° C., and the resulting intermediates can be chlorinated in situ using an appropriate chlorinating reagent, for example phosphorus oxychloride. Suitable acids are, for example, hydrohalic acids, such as HCl, or else Lewis acids, such as, for example, AlCl₃ or BF₃ (cf. U.S. Pat. No. 5,095,113).

Intermediates of the formula (X) where Z¹, Z⁴=trihalomethyl can be prepared by reacting the corresponding trihaloacetonitriles with a carbonitrile of the formula (IX). The reaction components are, for example, reacted with acid catalysis in an inert organic solvent such as toluene, chlorobenzene or chlorinated hydrocarbons at temperatures between −40□C and the boiling point of the solvent, preferably at from −10□C to 30□C. Examples of suitable acids are hydrohalic acids such as HCl or else Lewis acids such as AlCl₃ or BF₃ (cf. EP-A-1 30 939).

Intermediates of the formula (IV) in which Z¹=(C₁-C₄)alkylmercapto or unsubstituted phenyl-(C₁-C₄)-alkylmercapto can be converted in an inert organic solvent, for example toluene, chlorobenzene, chlorinated hydrocarbons or others, at temperatures between −40□C and the boiling point of the solvent, preferably at from 20□C to 80□C, using an appropriate chlorinating reagent, for example elemental chlorine or phosphorus oxychloride, to more reactive chlorotriazines of the formula (IV), in which Z¹ is Cl (cf. J. K Chakrabarti, D. E. Tupper; Tetrahedron 1975, 31(16), 1879-1882).

Intermediates of the formula (IV) in which Z¹=(C₁-C₄)alkylmercapto or unsubstituted or substituted phenyl-(C₁-C₄)-alkylmercapto or (C₁-C₄)alkyl-phenylthio can be oxidized in an appropriate solvent, for example chlorinated hydrocarbons, acetic acid, water, alcohols, acetone or mixtures thereof, at temperatures between 0□C and the boiling point of the solvent, preferably from 20□C to 80□C, using a suitable oxidation reagent such as m-chloroperbenzoic acid, hydrogen peroxide, potassium peroxomonosulfate (cf.: T. A. Riley, W. J. Henney, N. K. Dalley, B. E. Wilson, R. K. Robins; J. Heterocyclic Chem.; 1986, 23 (6), 1706-1714).

For the preparation of the acid addition salts of the compounds of the formula (I), suitable acids are the following: hydrohalic acids such as hydrochloric acid or hydrobromic acid, and also phosphoric acid, nitric acid, sulfuric acid, mono- or bifunctional carboxylic acids and hydroxycarboxylic acids such as acetic acid, maleic acid, succinic acid, fumaric acid, tartaric acid, citric acid, salicylic acid, sorbic acid or lactic acid, and also sulfonic acids such as p-toluenesulfonic acid or 1,5-naphthalenedisulfonic acid. The acid addition compounds of the formula (I) can be obtained in a simple manner by the customary methods of forming salts, for example by dissolving a compound of the formula (I) in an appropriate organic solvent such as, for example, methanol, acetone, methylene chloride or benzine and adding the acid at temperatures from 0 to 100□C, and can be isolated in a known manner, for example by filtration, and can if desired be purified by washing with an inert organic solvent.

The base addition salts of the compounds of formula (I) are preferably prepared in inert polar solvents such as, for example, water, methanol or acetone at temperatures from 0 to 100° C. Examples of suitable bases for preparing the salts according to the invention are alkali metal carbonates, such as potassium carbonate, alkali metal hydroxides and alkaline earth metal hydroxides, for example NaOH or KOH, alkali metal hydrides and alkaline earth metal hydrides, for example NaH, alkali metal alkoxides and alkaline earth metal alkoxides, for example sodium methoxide and potassium tert-butoxide, or ammonia or ethanolamine.

By the “inert solvents” mentioned in the above process variants, solvents are meant which are in each case inert under the respective reaction conditions but which need not be inert under any reaction conditions.

The novel compounds of the formulae have an outstanding herbicidal activity against a broad spectrum of economically important monocotyledonous and dicotyledonous weeds. The active compounds also act effectively against difficult-to-control perennial weeds which produce shoots from rhizomes, rootstocks or other perennial organs. In this context it does not matter whether the substances are applied before sowing, pre-emergence or post-emergence. Individually, examples may be mentioned of some representatives of the monocotyledonous and dicotyledonous weed flora which can be controlled by the compounds according to the invention, without such mention being intended to restrict the invention to specific species. Examples of monocotyledonous weed species against which the active compounds act effectively are Avena, Lolium, Alopecurus, Phalaris, Echinochloa, Digitaria, Setaria and Cyperus species from the annual group and examples of perennial species are Agropyron, Cynodon, Imperata and Sorghum and also perennial Cyperus species. In the case of dicotyledonous weed species, the spectrum of action extends to species such as, for example, Galium, Viola, Veronica, Lamium, Stellaria, Amaranthus, Sinapis, lpomoea, Matricaria, Abutilon and Sida among the annuals and Convolvulus, Cirsium, Rumex and Artemisia among the perennial weeds. The novel active compounds also display outstanding control of weeds which occur under the specific growing conditions in rice, examples being Echinochloa, Sagittaria, Alisma, Eleocharis, Scirpus and Cyperus.

Where the novel compounds are applied to the surface of the soil before germination, either the weed seedlings are prevented completely from emerging, or the weeds grow until they reach the cotyledon stage, but then stop growing and, finally, die off completely after three to four weeks have elapsed. Where the active compounds are applied post-emergence to the green parts of plants, there is likewise a very rapid and drastic termination of growth after treatment, and the weed plants remain at the growth stage they were at at the time of application, or die off completely after a certain time, so that in this manner competition from weeds, which is damaging to the crop plants, is eliminated very early on and in a sustained manner.

Even though the novel compounds have an excellent herbicidal activity with respect to monocotyledonous and dicotyledonous weeds, crop plants of economically important crops, for example wheat, barley, rye, rice, maize, sugar beet, cotton and soya, suffer only minimal or zero damage. For these reasons the present compounds are highly suitable for the selective control of unwanted plant growth in crops of agriculturally useful plants or in plantations of ornamentals.

Owing to their herbicidal and plant growth-regulatory properties, the active compounds can also be employed for controlling harmful plants in crops of genetically engineered plants which are known or still to be developed. The transgenic plants generally have particularly advantageous properties, for example resistance to certain pesticides, in particular certain herbicides, resistance to plant diseases or causative organisms of plant diseases, such as certain insects or microorganisms such as fungi, bacteria or viruses. Other particular properties relate, for example, to the quantity, quality, storage-stability, composition and to specific ingredients of the harvested product. Thus, transgenic plants having an increased starch content or a modified quality of the starch or those having a different fatty acid composition of the harvested product are known.

The use of the compounds according to the invention of the formula I or their salts in economically important transgenic crops of useful and ornamental plants, for example of cereal, such as wheat, barley, rye, oats, millet, rice, maniok and corn, or else in crops of sugarbeet, cotton, soya, rapeseed, potato, tomato, pea and other vegetable species is preferred. The compounds of the formula I can preferably be used as herbicides in crops of useful plants which are resistant or which have been made resistant by genetic engineering toward the phytotoxic effects of the herbicides.

Conventional ways for preparing novel plants which have modified properties compared to known plants comprise, for example, traditional cultivation methods and the generation of mutants. Alternatively, novel plants having modified properties can be generated with the aid of genetic engineering methods (see, for example, EP-A 0 221 044, EP-A 0 131 624). For example, there have been described several cases of

genetically engineered changes in crop plants in order to modify the starch synthesized in the plants (for example WO 92/11376, WO 92/14827, WO 91/19806),

transgenic crop plants which are resistant to certain herbicides of the glufosinate type (cf., for example, EP-A 0 242 236, EP-A 0 242 246) or glyphosate type (WO 92/00377), or of the sulfonylurea type (EP-A 0 257 993, U.S. Pat. No. 5,013,659),

transgenic crop plants, for example cotton, having the ability to produce Bacillus thuringiensis toxins (Bt toxins) which give the plants resistance to certain pests (EP-A 0 142 924, EP-A 0 193 259),

transgenic crop plants having a modified fatty add composition (WO 91113972).

Numerous molecular biological techniques which allow the preparation of novel transgenic plants having modified properties are known in principle; see, for example, Sambrook et al., 1989, Molecular Cloning, A Laboratory Manual, 2nd ed. Cold Spring Harbor Laboratory Press, Cold Spring Harbor, N.Y.; or Winnacker “Gene und Klone” [Genes and Clones], VCH Weinheim, 2nd edition 1996, or Christou, “Trends in Plant Science” 1 (1996) 423-431.

In order to carry out such genetic engineering manipulations, it is possible to introduce nucleic acid molecules into plasmids which allow a mutagenesis or a change in the sequence to occur by recombination of DNA sequences. Using the abovementioned standard processes it is possible, for example, to exchange bases, to remove partial sequences or to add natural or synthetic sequences. To link the DNA fragments with each other, it is possible to attach adaptors or linkers to the fragments.

Plant cells having a reduced activity of a gene product can be prepared, for example, by expressing at least one appropriate antisense-RNA, a sense-RNA to achieve a cosuppression effect, or by expressing at least one appropriately constructed ribozyme which specifically cleaves transcripts of the abovementioned gene product.

To this end it is possible to employ both DNA molecules which comprise the entire coding sequence of a gene product including any flanking sequences that may be present, and DNA molecules which comprise only parts of the coding sequence, it being necessary for these parts to be long enough to cause an antisense effect in the cells. It is also possible to use DNA sequences which have a high degree of homology to the coding sequences of a gene product but which are not entirely identical.

When expressing nucleic acid molecules in plants, the synthesized protein can be localized in any desired compartment of the plant cells. However, to achieve localization in a certain compartment, it is, for example, possible to link the coding region with DNA sequences which ensure localization in a certain compartment. Such sequences are known to the person skilled in the art (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106).

The transgenic plant cells can be regenerated to whole plants using known techniques. The transgenic plants can in principle be plants of any desired plant species, i.e. both monocotyledonous and dicotyledonous plants.

In this manner, it is possible to obtain transgenic plants which have modified properties by overexpression, suppression or inhibition of homologous (=natural) genes or gene sequences or by expression of heterologous (=foreign) genes or gene sequences.

The compounds according to the invention can preferably be used in transgenic crops which are resistant to herbicides from the group consisting of the sulfonylureas, glufosinate-ammonium or glyphosate-isopropylammonium and analogous active compounds.

When using the active compounds according to the invention in transgenic crops, in addition to the effects against harmful plants which can be observed in other crops, there are frequently effects which are specific for the application in the respective transgenic crop, for example a modified or specifically broadened spectrum of weeds which can be controlled, modified application rates which can be used for the application, preferably good combinability with the herbicides to which the transgenic crops are resistant, and an effect on the growth and the yield of the transgenic crop plants.

The invention therefore also provides for the use of the compounds according to the invention as herbicides for controlling harmful plants in transgenic crop plants.

Furthermore, the substances according to the invention exhibit outstanding growth-regulatory properties in crop plants. They intervene with a regulatory action in the endogenous plant metabolism and can therefore be employed for the targeted control of plant contents and for facilitating the harvest, for example by provoking desiccation and stunted growth. Moreover, they are also suitable for generally controlling and inhibiting unwanted vegetative growth without killing the plants in doing so. In the case of numerous monocotyledonous and dicotyledonous crops, inhibition of vegetative growth plays an important role, since it allows lodging to be reduced or prevented completely.

The invention also provides herbicidal and plant growth-regulatory compositions comprising compounds of the formula I. The novel compounds can be applied in the form of customary formulations, known to the person skilled in the art, such as wettable powders, emulsifiable concentrates, sprayable solutions, dusts or granules.

The compounds of the formula I can be formulated in various ways depending on the prevailing biological and/or chemico-physical parameters. Examples of suitable formulation options are: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), oil- or water-based dispersions, oil-miscible solutions, capsule suspensions (CS), dusts (DP), seed-dressing compositions, granules for broadcasting and soil application, granules (GR) in the form of microgranules, spray granules, coating granules and adsorption granules, water-dispersible granules (WG), water-soluble granules (SG), ULV formulations, microcapsules and waxes. These individual formulation types are known in principle and are described, for example, in Winnacker-Küchler, “Chemische Technologie” [Chemical Technology], Volume 7, C. Hauser Verlag Munich, 4th. Edition, 1986; Wade van Valkenburg, “Pesticide Formulations”, Marcel Dekker, N.Y., 1973; K. Martens, “Spray Drying” Handbook, 3rd Ed. 1979, G. Goodwin Ltd. London.

The necessary formulation auxiliaries, such as inert materials, surfactants, solvents and other additives, are likewise known and are described, for example, in Watkins, “Handbook of Insecticide Dust Diluents and Carriers”, 2nd Ed., Darland Books, Caldwell N.J., H.v. Olphen, “Introduction to Clay Colloid Chemistry”; 2nd Ed., J. Wiley & Sons, N.Y.; C. Marsden, “Solvents Guide”; 2nd Ed., Interscience, N.Y. 1963; McCutcheon's “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964; Schonfeldt, “Grenzflächenaktive Äthylenoxidaddukte” [Surface-active ethylene oxide adducts], Wiss. Verlagsgesell., Stuttgart 1976; Winnacker-Küchler, “Chemische Technologie” [Chemical Technology], Volume 7, C. Hauser Verlag Munich, 4th Edition 1986.

Wettable powders are preparations which are uniformly dispersible in water and which contain, in addition to the active compound and as well as a diluent or inert substance, surfactants of the ionic and/or nonionic type (wetting agents, dispersants), for example polyethoxylated alkyl phenols, polyethoxylated fatty alcohols, polyethoxylated fatty amines, fatty alcohol polyglycol ethersulfates, alkanesulfonates, alkylbenzenesulfonates, sodium ligninsulfonate, sodium 2,2′-dinaphthylmethane-6,6′-disulfonate, sodium dibutylnaphthalenesulfonate or else sodium oleoylmethyltaurinate. To prepare the wettable powders, the herbicidally active compounds are finely ground, for example in customary apparatus such as hammer mills, fan mills and air-jet mills, and are mixed simultaneously or subsequently with the formulation auxiliaries.

Emulsifiable concentrates are prepared by dissolving the active compound in an organic solvent, for example butanol, cyclohexanone, dimethylformamide, xylene or else relatively high-boiling aromatic compounds or hydrocarbons or mixtures of the organic solvents, with the addition of one or more surfactants of the ionic and/or nonionic type (emulsifiers). Examples of emulsifiers which can be used are calcium alkylarylsulfonates, such as Ca dodecylbenzenesulfonate, or nonionic emulsifiers, such as fatty acid polyglycol esters, alkylaryl polyglycol ethers, fatty alcohol polyglycol ethers, propylene oxide-ethylene oxide condensation products, alkyl polyethers, sorbitan esters, for example sorbitan fatty acid esters or polyoxyethylene sorbitan esters, for example polyoxyethylene sorbitan fatty acid esters.

Dusts are obtained by grinding the active compound with finely divided solid substances, for example talc, natural clays, such as kaolin, bentonite and pyrophyllite, or diatomaceous earth.

Suspension concentrates can be water- or oil-based. They can be prepared, for example, by wet milling using commercially customary bead mills, with or without the addition of surfactants as already mentioned above, for example, in the case of the other formulation types.

Emulsions, for example oil-in-water emulsions (EW), can be prepared for example by means of stirrers, colloid mills and/or static mixers using aqueous organic solvents and, if desired, surfactants as already mentioned above, for example, in the case of the other formulation types.

Granules can be prepared either by spraying the active compound onto adsorptive, granulated inert material or by applying active-compound concentrates to the surface of carriers such as sand, kaolinites or granulated inert material, by means of adhesive binders, for example polyvinyl alcohol, sodium polyacrylate or else mineral oils. Suitable active compounds can also be granulated in the manner which is customary for the preparation of fertilizer granules, if desired as a mixture with fertilizers.

Water-dispersible granules are generally prepared by the customary processes, such as spray-drying, fluidized-bed granulation, disk granulation, mixing using high-speed mixers, and extrusion without solid inert material.

For the preparation of disk fluidized-bed, extruder and spray granules, see for example processes in “Spray-Drying Handbook” 3rd ed. 1979, G. Goodwin Ltd., London; J. E. Browning, “Agglomeration”, Chemical and Engineering 1967, pages 147 ff.; “Perry's Chemical Engineer's Handbook”, 5th Ed., McGraw-Hill, New York 1973, pp. 8-57.

For further details on the formulation of crop protection products, see for example G. C. Klingman, “Weed Control as a Science”, John Wiley and Sons Inc., New York, 1961, pages 81-96 and J. D. Freyer, S. A. Evans, “Weed Control Handbook”, 5th Ed., Blackwell Scientific Publications, Oxford, 1968, pages 101-103.

The agrochemical formulations generally contain from 0.1 to 99% by weight, in particular from 0.1 to 95% by weight, of active compound of the formula I. In wettable powders the concentration of active compound is, for example, from about 10 to 90% by weight, the remainder to 100% by weight consisting of customary formulation constituents. In emulsifiable concentrates the concentration of active compound can be from about 1 to 90%, preferably from 5 to 80%, by weight. Formulations in the form of dusts contain from 1 to 30% by weight of active compound, preferably most commonly from 5 to 20% by weight of active compound, while sprayable solutions contain from about 0.05 to 80%, preferably from 2 to 50%, by weight of active compound. In the case of water-dispersible granules the content of active compound depends partly on whether the active compound is in liquid or solid form and on the granulation auxiliaries, fillers, etc. that are used. In water-dispersible granules the content of active compound is, for example, between 1 and 95% by weight, preferably between 10 and 80% by weight.

In addition, said formulations of active compound may comprise the tackifiers, wetting agents, dispersants, emulsifiers, penetrants, preservatives, antifreeze agents, solvents, fillers, carriers, colorants, antifoams, evaporation inhibitors and pH and viscosity regulators which are customary in each case.

Based on these formulations it is also possible to produce combinations with other pesticidally active substances, for example insecticides, acaricides, herbicides and fungicides, and also with safeners, fertilizers and/or growth regulators, for example in the form of a ready-mix or tank mix.

Suitable active ingredients which can be combined with the active ingredients according to the invention in mixed formulations or in a tank mix are, for example, known active ingredients as described for example in Weed Research 26, 441 445 (1986), or “The Pesticide Manual”, 11th edition, The British Crop Protection Council and the Royal Soc. of Chemistry, 1997 and in the literature cited therein. For example the following active ingredients may be mentioned as herbicides which are known and which can be combined with the compounds of the formula I (note: the compounds are either named by the “common name” in accordance with the International Organization for Standardization (ISO) or by the chemical names, if appropriate together with a customary code number): acetochlor; acifluorfen; aclonifen; AKH 7088, i.e. [[[1-[5-[2-chloro-4-(trifluoromethyl)phenoxy]-2-nitrophenyl]-2-methoxyethylidene]amino]oxy]acetic acid and its methyl ester; alachlor; alloxydim; ametryn; amidosulfuron; amitrol; AMS, i.e. ammonium sulfamate; anilofos; asulam; atrazine; azimsulfurone (DPX-A8947); aziprotryn; barban; BAS 516H, i.e. 5-fluoro-2-phenyl4H-3,1-benzoxazin4-one; benazolin; benfluralin; benfuresate; bensulfuron-methyl; bensulide; bentazone; benzofenap; benzofluor; benzoylprop-ethyl; benzthiazuron; bialaphos; bifenox; bromacil; bromobutide; bromofenoxim; bromoxynil; bromuron; buminafos; busoxinone; butachlor; butamifos; butenachlor; buthidazole; butralin; butylate; cafenstrole (CH-900); carbetamide; cafentrazone (ICI-A0051); CDM, i.e. 2-chloro-N,N-di-2-propenylacetamide; CDEC, i.e. 2-chloroallyl diethyldithiocarbamate; chlomethoxyfen; chloramben; chlorazifop-butyl, chlormesulon (ICI-A0051); chlorbromuron; chlorbufam; chlorfenac; chlorflurecol-methyl; chloridazon; chlorimuron ethyl; chlornitrofen; chlorotoluron; chloroxuron; chlorpropham; chlorsulfuron; chlorthal-dimethyl; chlorthiamid; cinmethylin; cinosulfuron; clethodim; clodinafop and its ester derivatives (for example clodinafop-propargyl); clomazone; clomeprop; cloproxydim; clopyralid; cumyluron (JC 940); cyanazine; cycloate; cyclosuffamuron (AC 104); cycloxydim; cycluron; cyhalofop and its ester derivatives (for example butyl ester, DEH-112); cyperquat; cyprazine; cyprazole; daimuron; 2,4-DB; dalapon; desmedipham; desmetryn; di-allate; dicamba; dichlobenil; dichlorprop; diclofop and its esters such as diclofop-methyl; diethatyl; difenoxuron; difenzoquat; diflufenican; dimefuron; dimethachlor; dimethametryn; dimethenamid (SAN-582H); dimethazone, clomazon; dimethipin; dimetrasulfuron, dinitramine; dinoseb; dinoterb; diphenamid; dipropetryn; diquat; dithiopyr; diuron; DNOC; eglinazine-ethyl; EL 77, i.e. 5-cyano-1-(1,1-dimethylethyl)-N-methyl-1H-pyrazole4-carboxamide; endothal; EPTC; esprocarb; ethalfluralin; ethametsulfuron-methyl; ethidimuron; ethiozin; ethofumesate; F5231, i.e. N-[2-chloro4-fluoro-5-[4-(3-fluoropropyl)4,5-dihydro-5-oxo-1H-tetrazol-1-yl]-phenyl]ethanesulfonamide; ethoxyfen and its esters (for example ethyl ester, HN-252); etobenzanid (HW 52); fenoprop; fenoxan, fenoxaprop and fenoxaprop-P and their esters, for example fenoxaprop-P-ethyl and fenoxaprop-ethyl; fenoxydim; fenuron; flamprop-methyl; flazasulfuron; fluazifop and fluazifop-P and their esters, for example fluazifop-butyl and fluazifop-P-butyl; fluchloralin; flumetsulam; flumeturon; flumiclorac and its esters (for example pentyl ester, S-23031); flumioxazin (S482); flumipropyn; flupoxam (KNW-739); fluorodifen; fluoroglycofen-ethyl; flupropacil (UBIC4243); fluridone; flurochloridone; fluroxypyr; flurtamone; fomesafen; fosamine; furyloxyfen; glufosinate; glyphosate; halosafen; halosulfuron and its esters (for example methyl ester, NC-319); haloxyfop and its esters; haloxyfop-P (=R-haloxyfop) and its esters; hexazinone; imazamethabenz-methyl; imazapyr; imazaquin and salts such as the ammonium salt; imazethamethapyr; imazethapyr; imazosulfuron; ioxynil; isocarbamid; isopropalin; isoproturon; isouron; isoxaben; isoxapyrifop; karbutilate; lactofen; lenacil; linuron; MCPA; MCPB; mecoprop; mefenacet; mefluidid; metamitron; metazachlor; methabenzthiazuron; metham; methazole; methoxyphenone; methyldymron; metobenzuron; metobromuron; metolachlor; metosulam (XRD 511); metoxuron; metribuzin; metsulfuron-methyl; MH; molinate; monalide; monocarbamide dihydrogensulfate; monolinuron; monuron; MT 128, i.e. 6-chloro-N-(3-chloro-2-propenyl)-5-methyl-N-phenyl-3-pyridazin-amine; MT 5950, i.e. N-[3-chloro-4-(1-methylethyl)-phenyl]-2-methyl-pentanamide; naproanilide; napropamide; naptalam; NC 310, i.e. 4-(2,4-dichlorobenzoyl)-1-methyl-5-benzyloxypyrazole; neburon; nicosulfuron; nipyraclophen; nitralin; nitrofen; nitrofluorfen; norflurazon; orbencarb; oryzalin; oxadiargyl (RP-020630); oxadiazon; oxyfluorfen; paraquat; pebulate; pendimethalin; perfluidone; phenisopham; phenmedipham; picloram; piperophos; piributicarb; pirifenop-butyl; pretilachlor; primisulfuron-methyl; procyazine; prodiamine; profluralin; proglinazine-ethyl; prometon; prometryn; propachlor; propanil; propaquizafop and its esters; propazine; propham; propisochlor; propyzamide; prosulfalin; prosulfocarb; prosulfuron (CGA-152005); prynachlor; pyrazolinate; pyrazon; pyrazosulfuron-ethyl; pyrazoxyfen; pyridate; pyrithiobac (KIH-2031); pyroxofop and its esters (for example propargyl ester); quinclorac; quinmerac; quinofop and its ester derivatives, quizalofop and quizalofop-P and their ester derivatives, for example quizalofop-ethyl; quizalofop-P-tefuryl and -ethyl; renriduron; rimsulfuron (DPX-E 9636); S 275, i.e. 2-[4-chloro-2-fluoro-5-(2-propynyloxy)phenyl]-4,5,6,7-tetrahydro-2H-indazole; secbumeton; sethoxydim; siduron; simazine; simetryn; SN 106279, i.e. 2-[7-[2-chloro-4-(trifluoromethyl)phenoxy]-2-naphthalenyl]oxy]propanoic acid and its methyl ester; sulfentrazon (FMC-97285, F-6285); sulfazuron; sulfometuron-methyl; sulfosate (ICI-A0224); TCA; tebutam (GCP-5544); tebuthiuron; terbacil; terbucarb; terbuchlor; terbumeton; terbuthylazine; terbutryn; TFH 450, i.e. N,N-diethyl-3-[(2-ethyl-6-methylphenyl)sulfonyl]-1H-1,2,4-triazole-1-carboxamide; thenylchlor (NSK-850); thiazafluron; thiazopyr (Mon-13200); thidiazimin (SN-24085); thifensulfuron-methyl; thiobencarb; tiocarbazil; tralkoxydim; tri-allate; triasulfuron; triazofenamide; tribenuron-methyl; triclopyr; tridiphane; trietazine; trifluralin; triflusulfuron and its esters (for example methyl ester, DPX-46037); trimeturon; tsitodef; vernolate; WL 110547, i.e. 5-phenoxy-1-[3-(trifluoromethyl)phenyl]-1H-tetrazole; UBH-509; D-489; LS 82-556; KPP-300; NC-324; NC-330; KH-218; DPX-N8189; SC-0774; DOWCO-535; DK-8910; V-53482; PP-600; MBH-001; KIH-9201; ET-751; KIH-6127 and KIH-2023.

For use, the formulations which are present in commercially available form are, if appropriate, diluted in the customary manner, for example using water in the case of wettable powders, emulsifiable concentrates, dispersions and water-dispersible granules. Products in the form of dusts, granules for soil application or broadcasting and sprayable solutions are usually not further diluted with other inert substances prior to use.

The application rate of the compounds of the formula I required varies with the external conditions, such as temperature, humidity, the nature of the herbicide used and the like. It can vary within wide limits, for example between 0.001 and 10.0 kg/ha or more of active substance, but it is preferably between 0.005 and 5 kg/ha.

The examples below illustrate the invention.

### A. CHEMICAL EXAMPLES

1. Preparation of 2-amino-4-fluoroisopropyl-6-[N-(1-methyl-3-phenoxyprop-1-yl)amino]-1,3,5-triazine (see Table 2, Example No. 368)

0.45 g (0.019 mol) of sodium was dissolved in 50 ml of anhydrous methanol. 1.85 g (0.006 mol) of 3-biguanido-1-phenoxybutane hydrochloride were added, followed by 1.3 g (0.010 mol) of ethyl fluoroisobutyrate. The mixture was heated at reflux for 3 h. The solvent was removed under reduced pressure and the residue was taken up in dichloromethane/water and extracted repeatedly. The organic phase was dried over MgSO₄ and filtered, and the solvent was removed under reduced pressure. Purification by silica gel column chromatography using ethyl acetate/hepane (1:1) gave 0.45 g (24%) of the title compound.

2. Preparation of 2-amino-4-isopropyl-6-{N-[1-methyl-2-(4-chlorobenzyl-oxy)ethyl]amino}-1,3,5-triazine (see Table 1, Example No. 166)

1.00 g (0.004 mol) of 2-aminopropyl 4-chlorobenzyl ether hydrochloride was initially charged together with 2.34 g (0.017 mol) of potassium carbonate and 0.73 g (0.004 mol) of 2-amino-4-chloro-4-isopropyl-1,3,5-triazine, and the mixture was heated at reflux for 3 h. The solvent was removed under reduced pressure and the residue was taken up in ethyl acetate/water and extracted repeatedly. The organic phase was dried over MgSO₄ and filtered, and the solvent was removed under reduced pressure. Purification by silica gel column chromatography using ethyl acetate/heptane (1:1) gave 1.20 g (89%) of the title compound.

3. Preparation of 2-amino-4-isopropyl-6-{N-[2-(3,4-dichlorobenzoyl-oxy)ethyl]amino}-1,3,5-triazine

0.5 g (0.0024 mol) of 3,4-dichlorobenzoyl chloride was initially charged in 30 ml of 1,4-dioxane, and 0.47 g (0.0024 mol) of 2-amino-4-isopropyl-6-[N-(2-hydroxyethyl)amino-1,3,5-triazine and 0.483 g (0.005 mol) of triethylamine were added. The mixture was heated at reflux for 4 h. The precipitated solid was filtered off, and the solvent was removed under reduced pressure. The residue was taken up in ethyl acetate/water and extracted repeatedly. The organic phase was dried over MgSO₄ and filtered, and solvent was removed under reduced pressure. This gave 0.65 g (73%) of the title compound.

The examples listed in the tables below were prepared by the methods mentioned above, or they can be obtained by the methods mentioned above.

The meanings of the abbreviations used in the tables are as follows:

| | | | | | |
|---|---|---|---|---|---|
| Et = | ethyl | Me = | methyl | c = | cyclo |
| n = | normal | i = | iso | s = | secondary |
| t = | tertiary | | | | |
| m.p. = | melting point | | | | |

An asymmetrical divalent group Y, such as Y═C(O)O (i.e. —CO—O—) is, in the context of the tables, a group where the linkage point of the group atom shown at the left is attached to the molecular moiety which is shown, in the formula (I), as being to the left of Y.

TABLE 1
Compounds of the formula (Ia) (=formula I where n = 1 and R¹, R², R⁵,
R⁷, R⁹, R¹⁰ are each H and aryl = phenyl which is substituted as shown in the table)

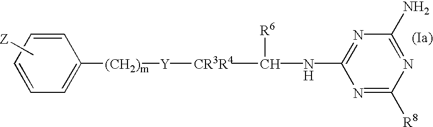

| No. | Z | m | Y | R³ | R⁴ | R⁶ | R⁸ | Phys. data |
|---|---|---|---|---|---|---|---|---|
| 1 | 2,4-Cl₂ | 0 | C(O)O | H | H | Me | i-Propyl | oil |
| 2 | 2,4-Cl₂ | 0 | C(O)O | H | H | Et | i-Propyl | oil |
| 3 | 2,4-Cl₂ | 0 | C(O)O | H | H | H | i-Propyl | oil |
| 4 | 2,4-Cl₂ | 0 | C(O)O | H | H | Me | CF₃ | |
| 5 | 2,4-F₂ | 0 | C(O)O | H | H | Me | i-Propyl | oil |
| 6 | 2,4-F₂ | 0 | C(O)O | H | H | Et | i-Propyl | oil |
| 7 | 2,4-F₂ | 0 | C(O)O | H | H | H | i-Propyl | oil |
| 8 | 2,4-F₂ | 1 | C(O)O | H | H | n-Butyl | CF₂CHF₂ | |
| 9 | 2,5-F₂ | 0 | C(O)O | H | H | Me | i-Propyl | oil |
| 10 | 2,5-F₂ | 0 | C(O)O | H | H | Et | i-Propyl | oil |
| 11 | 2,5-F₂ | 0 | C(O)O | H | H | H | i-Propyl | oil |
| 12 | 2,5-F₂ | 1 | C(O)O | H | H | H | CF(CH₃)₂ | |
| 13 | 2,6-F₂ | 0 | C(O)O | H | H | Me | i-Propyl | oil |
| 14 | 2,6-F₂ | 0 | C(O)O | H | H | Et | i-Propyl | oil |
| 15 | 2,6-F₂ | 0 | C(O)O | H | H | H | i-Propyl | oil |
| 16 | 2,6-F₂ | 1 | C(O)O | H | H | Me | CF₃ | |
| 17 | 2-CF₃ | 0 | C(O)O | H | H | Et | i-Propyl | |
| 18 | 2-Cl | 0 | C(O)O | H | H | Me | i-Propyl | oil |
| 19 | 2-Cl | 0 | C(O)O | H | H | Et | i-Propyl | oil |
| 20 | 2-Cl | 0 | C(O)O | H | H | H | i-Propyl | oil |
| 21 | 2-Cl | 0 | C(O)O | H | H | n-Butyl | CF₂CHF₂ | |
| 22 | 2-Cl,4-F | 0 | C(O)O | H | H | Me | i-Propyl | oil |
| 23 | 2-Cl,4-F | 0 | C(O)O | H | H | Et | i-Propyl | oil |
| 24 | 2-Cl,4-F | 0 | C(O)O | H | H | H | i-Propyl | oil |
| 25 | 2-Cl,4-F | 0 | C(O)O | Me | H | Me | CF(CH₃)₂ | |
| 26 | 2-Cl,4-NO₂ | 0 | C(O)O | H | H | Me | i-Propyl | oil |
| 27 | 2-Cl,4-NO₂ | 0 | C(O)O | H | H | Et | i-Propyl | oil |
| 28 | 2-Cl,4-NO₂ | 0 | C(O)O | H | H | H | i-Propyl | oil |
| 29 | 2-Cl,4-NO₂ | 0 | C(O)O | H | H | 1-Butyl | i-Propyl | |
| 30 | 2-Cl,5-NO₂ | 0 | C(O)O | H | H | Me | i-Propyl | oil |
| 31 | 2-Cl,5-NO₂ | 0 | C(O)O | H | H | Et | i-Propyl | oil |
| 32 | 2-Cl,5-NO₂ | 0 | C(O)O | H | H | H | i-Propyl | oil |
| 33 | 2-Cl,5-NO₂ | 0 | C(O)O | H | H | Me | CF(CH₃)₂ | |
| 34 | 2-F | 0 | C(O)O | H | H | Me | i-Propyl | oil |
| 35 | 2-F | 0 | C(O)O | H | H | Et | i-Propyl | oil |
| 36 | 2-F | 0 | C(O)O | H | H | H | i-Propyl | oil |
| 37 | 2-F | 0 | C(O)O | Me | H | CH₂OEt | CF(CH₃)₂ | |
| 38 | 2-F,3-CF₃ | 0 | C(O)O | H | H | Et | i-Propyl | |
| 39 | 2-F,4-CF₃ | 0 | C(O)O | H | H | Et | i-Propyl | |
| 40 | 2-F,5-CF₃ | 0 | C(O)O | H | H | Et | i-Propyl | |
| 41 | 2-Me | 0 | C(O)O | H | H | Me | i-Propyl | oil |
| 42 | 2-Me | 0 | C(O)O | H | H | Et | i-Propyl | oil |
| 43 | 2-Me | 0 | C(O)O | H | H | H | i-Propyl | oil |
| 44 | 2-Me | 2 | C(O)O | H | H | CH₂-c-Propyl | CF(CH₃)₂ | |
| 45 | 2-OAc | 0 | C(O)O | H | H | Me | i-Propyl | oil |
| 46 | 2-OAc | 0 | C(O)O | H | H | Et | i-Propyl | oil |
| 47 | 2-OAc | 0 | C(O)O | H | H | H | i-Propyl | oil |
| 48 | 2-OAc | 1 | C(O)O | Me | H | c-Pentyl | CHFCH₃ | |
| 49 | 3,4-Cl₂ | 0 | C(O)O | H | H | Me | i-Propyl | oil |
| 50 | 3,4-Cl₂ | 0 | C(O)O | H | H | Et | i-Propyl | oil |
| 51 | 3,4-Cl₂ | 0 | C(O)O | H | H | H | i-Propyl | oil |
| 52 | 3,4-Cl₂ | 0 | C(O)O | H | H | s-Butyl | CHFCF₃ | |
| 53 | 3,5-(CF₃)₂ | 0 | C(O)O | H | H | Et | i-Propyl | |
| 54 | 3,5-Cl₂ | 0 | C(O)O | H | H | Me | i-Propyl | oil |
| 55 | 3,5-Cl₂ | 0 | C(O)O | H | H | Et | i-Propyl | oil |
| 56 | 3,5-Cl₂ | 0 | C(O)O | H | H | H | i-Propyl | oil |
| 57 | 3,5-Cl₂ | 0 | C(O)O | H | H | CH₂OMe | CHFCH₃ | |
| 58 | 3,5-F₂ | 1 | SO₂ | Me | H | Me | CF₂CHF₂ | |
| 59 | 3,5-F₂ | 2 | 0 | H | H | Et | CF₂CHF₂ | |
| 60 | 3,5-F₂ | 1 | 0 | H | H | c-Butyl | CF₂CHF₂ | |
| 61 | 3,5-F₂ | 1 | 0 | H | H | H | CF₂CHF₂ | |
| 62 | 3,5-Me₂ | 1 | C(O)O | Et | H | H | CF₂CHF₂ | |
| 63 | 3,5-Me₂ | 1 | 0 | H | H | Me | i-Propyl | m.p. 80° C. |
| 64 | 3,5-Me₂ | 1 | 0 | H | H | Me | CF(CH₃)₂ | resin |
| 65 | 3,5-Me₂ | 1 | 0 | Me | H | Et | CHFCH₃ | |
| 66 | 3,5-Me₂ | 1 | 0 | H | H | c-Butyl | CF₂CHF₂ | |
| 67 | 3,5-Me₂ | 1 | 0 | H | H | c-Pentyl | CF(CH₃)₂ | |
| 68 | 3,5-Me₂ | 1 | 0 | Et | H | H | CF₂CHF₂ | |
| 69 | 3,5-(OMe)₂ | 1 | C(O)O | Me | H | H | CHFCH₃ | |
| 70 | 3,5-(OMe)₂ | 1 | 0 | H | H | Me | CF₃ | |
| 71 | 3,5-(OMe)₂ | 1 | S | Me | H | Et | CF(CH₃)₂ | |
| 72 | 3,5-(OMe)₂ | 1 | SO₂ | H | H | c-Butyl | CHFCH₃ | |
| 73 | 3,5-(OMe)₂ | 1 | O | H | H | c-Pentyl | i-Propyl | |
| 74 | 3,5-(OMe)₂ | 1 | SO₂ | Me | H | H | CHFCH₃ | |
| 75 | 3-Br | 1 | O | H | H | Me | i-Propyl | resin |
| 76 | 3-Br | 1 | O | H | H | Me | CF(CH₃)₂ | m.p. 101° C. |
| 77 | 3-Br | 1 | 0 | H | H | Me | CF₂CHF₂ | m.p. 128° C. |
| 78 | 3-CF₃ | 0 | C(O)O | H | H | Me | i-Propyl | oil |
| 79 | 3-CF₃ | 0 | C(O)O | H | H | Et | i-Propyl | oil |
| 80 | 3-CF₃ | 0 | C(O)O | H | H | H | i-Propyl | oil |
| 81 | 3-CF₃ | 1 | SO₂ | H | H | Et | CF₃ | |
| 82 | 3-CF₃ | 1 | O | H | H | c-Propyl | CHFCH₃ | |
| 83 | 3-CF₃ | 2 | O | H | H | c-Butyl | i-Propyl | |
| 84 | 3-Cl | 0 | C(O)O | H | H | Me | i-Propyl | oil |
| 85 | 3-Cl | 0 | C(O)O | H | H | Et | i-Propyl | oil |
| 86 | 3-Cl | 0 | C(O)O | H | H | H | i-Propyl | oil |
| 87 | 3-Cl | 0 | C(O)O | H | H | CF₃ | CF₂CHF₂ | |
| 88 | 3-Cl | 1 | C(O)O | H | H | H | CF(CH3)₂ | |
| 89 | 3-Cl | 1 | O | H | H | Me | i-Propyl | resin |
| 90 | 3-Cl | 1 | O | H | H | Me | CF(CH₃)₂ | resin |
| 91 | 3-Cl | 1 | O | H | H | Me | CF₂CHF₂ | m.p. 103° C. |
| 92 | 3-Cl | 1 | O | H | H | Et | CF₃ | |
| 93 | 3-Cl | 1 | SO₂ | H | H | c-Butyl | CF(CH₃)₂ | |
| 94 | 3-Cl | 1 | SO₂ | H | H | c-Pentyl | CF₂CHF₂ | |
| 95 | 3-Cl | 1 | SO₂ | H | H | H | CF(CH₃)₂ | |
| 96 | 3-Cl,5-Me | 1 | O | c-Propyl | H | Et | CF₂CHF₂ | |
| 97 | 3-Cl,5-Me | 1 | O | H | H | c-Propyl | CF₃ | |
| 98 | 3-Cl,5-Me | 1 | O | H | H | c-Butyl | CHFCH₃ | |
| 99 | 3-F | 0 | C(O)O | H | H | Me | i-Propyl | oil |
| 100 | 3-F | 0 | C(O)O | H | H | Et | i-Propyl | oil |
| 101 | 3-F | 0 | C(O)O | H | H | H | i-Propyl | oil |
| 102 | 3-F | 0 | C(O)O | H | H | i-Propyl | i-Propyl | |
| 103 | 3-F | 1 | C(O)O | c-Propyl | H | H | i-Propyl | |
| 104 | 3-F | 1 | O | H | H | Me | i-Propyl | |
| 105 | 3-F | 2 | O | H | H | Et | i-Propyl | |
| 106 | 3-F | 2 | O | H | H | c-Butyl | i-Propyl | |
| 107 | 3-F | 2 | O | c-Propyl | H | H | i-Propyl | |
| 108 | 3-F,4-CF₃ | 0 | C(O)O | H | H | Et | i-Propyl | |
| 109 | 3-F,5-CF₃ | 0 | C(O)O | H | H | Et | i-Propyl | |
| 110 | 3-F,5-Me | 2 | O | H | H | Me | i-Propyl | |
| 111 | 3-F,5-Me | 1 | O | H | H | Et | CHFCH₃ | |
| 112 | 3-F,5-Me | 2 | O | H | H | c-Propyl | i-Propyl | |
| 113 | 3-F,5-Me | 1 | S | H | H | c-Butyl | CF(CH₃)₂ | |
| 114 | 3-F,5-OMe | 2 | S | H | H | Me | CF₂CHF₂ | |
| 115 | 3-F,5-OMe | 1 | O | H | H | Et | CF(CH₃)₂ | |
| 116 | 3-F,5-OMe | 2 | S | H | H | c-Propyl | CF₂CHF₂ | |
| 117 | 3-F,5-OMe | 1 | SO₂ | H | H | c-Butyl | CF₃ | |
| 118 | 3-Me | 1 | C(O)O | H | H | H | CF₃ | |
| 119 | 3-Me | 1 | O | H | H | Me | i-Propyl | resin |
| 120 | 3-Me | 1 | O | H | H | Me | CF(CH₃)₂ | m.p. 112° C. |
| 121 | 3-Me | 1 | O | H | H | Me | CF₂CHF₂ | resin |
| 122 | 3-Me | 1 | O | H | H | Et | CF(CH₃)₂ | |
| 123 | 3-Me | 1 | O | H | H | c-Butyl | CF₃ | |
| 124 | 3-Me | 1 | O | H | H | c-Pentyl | CF₂CHF₂ | |
| 125 | 3-Me | 1 | O | H | H | H | CF₃ | |
| 126 | 3-Me,4-F | 2 | O | H | H | Me | CHFCH₃ | |
| 127 | 3-Me,4-F | 2 | SO₂ | H | H | Et | CF(CH₃)₂ | |
| 128 | 3-Me,4-F | 1 | O | H | H | c-Propyl | CHFCH₃ | |
| 129 | 3-Me,4-F | 1 | O | H | H | c-Butyl | CF(CH₃)₂ | |
| 130 | 3-Me,4-F | 1 | O | c-Propyl | H | H | CF(CH₃)₂ | |
| 131 | 3-OCF₃ | 0 | C(O)O | H | H | Me | i-Propyl | oil |
| 132 | 3-OCF₃ | 0 | C(O)O | H | H | Et | i-Propyl | oil |
| 133 | 3-OCF₃ | 0 | C(O)O | H | H | H | i-Propyl | oil |
| 134 | 3-OCF₃ | 0 | C(O)O | H | H | Me | CF(CH₃)₂ | |
| 135 | 3-OEt | 2 | O | c-Propyl | H | Me | CF(CH₃)₂ | |
| 136 | 3-OEt | 2 | S | H | H | Et | i-Propyl | |
| 137 | 3-OEt | 2 | O | Et | H | c-Propyl | CF(CH₃)₂ | |
| 138 | 3-OEt | 1 | O | Me | H | c-Butyl | CF₃ | |
| 139 | 3-OMe | 1 | C(O)O | H | H | H | i-Propyl | |
| 140 | 3-OMe | 1 | S | H | H | Me | CF(CH₃)₂ | |
| 141 | 3-OMe | 1 | O | H | H | Et | CF₃ | |
| 142 | 3-OMe | 1 | O | H | H | c-Butyl | i-Propyl | |
| 143 | 3-OMe | 1 | O | c-Propyl | H | c-Pentyl | CHFCH₃ | |
| 144 | 3-OMe | 1 | O | H | H | H | i-Propyl | |
| 145 | 3-OMe,5-Cl | 2 | 0 | H | H | Et | CHFCH₃ | |
| 146 | 3-OMe,5-Cl | 2 | S | H | H | c-Butyl | CHFCH₃ | |
| 147 | 3-OMe,5-Cl | 2 | S | Me | Me | H | CHFCH₃ | |
| 148 | 4-Br | 0 | C(O)O | H | H | Me | i-Propyl | oil |
| 149 | 4-Br | 0 | C(O)O | H | H | Et | i-Propyl | oil |
| 150 | 4-Br | 0 | C(O)O | H | H | H | i-Propyl | oil |
| 151 | 4-Br | 1 | C(O)O | Me | H | c-Pentyl | CHFCH₃ | |
| 152 | 4-Br | 1 | O | H | H | Me | i-Propyl | m.p. 82° C. |
| 153 | 4-Br | 1 | O | H | H | Me | CF(CH₃)₂ | resin |
| 154 | 4-Br | 1 | O | H | H | Me | CF₂CHF₂ | m.p. 114° C. |
| 155 | 4-CF₃ | 0 | C(O)O | H | H | Me | i-Propyl | oil |
| 156 | 4-CF₃ | 0 | C(O)O | H | H | Et | i-Propyl | oil |
| 157 | 4-CF₃ | 0 | C(O)O | H | H | H | i-Propyl | oil |
| 158 | 4-CF₃ | 0 | C(O)O | H | H | Me | i-Propyl | oil |
| 159 | 4-CF₃ | 0 | C(O)O | H | H | Et | i-Propyl | oil |
| 160 | 4-CF₃ | 0 | C(O)O | H | H | H | i-Propyl | oil |
| 161 | 4-CF₃ | 0 | C(O)O | H | H | CH₂-c-Butyl | CF₃ | |
| 162 | 4-Cl | 0 | C(O)O | H | H | Me | i-Propyl | oil |
| 163 | 4-Cl | 0 | C(O)O | H | H | Et | i-Propyl | oil |
| 164 | 4-Cl | 0 | C(O)O | H | H | H | i-Propyl | oil |
| 165 | 4-Cl | 0 | C(O)O | H | H | CH₂F | CHFCH₃ | |
| 166 | 4-Cl | 1 | O | H | H | Me | i-Propyl | resin |
| 167 | 4-Cl | 1 | O | H | H | Me | CF(CH₃)₂ | resin |
| 168 | 4-Cl | 1 | 0 | H | H | Me | CF₂CHF₂ | m.p. 104° C. |
| 169 | 4-F | 0 | C(O)O | H | H | Me | i-Propyl | oil |
| 170 | 4-F | 0 | C(O)O | H | H | Et | i-Propyl | oil |
| 171 | 4-F | 0 | C(O)O | H | H | H | i-Propyl | oil |
| 172 | 4-F | 1 | C(O)O | H | H | t-Butyl | i-Propyl | |
| 173 | 4-F,5-CF₃ | 0 | C(O)O | H | H | Et | i-Propyl | |
| 174 | 4-I | 0 | C(O)O | H | H | Me | i-Propyl | oil |
| 175 | 4-I | 0 | C(O)O | H | H | Et | i-Propyl | oil |
| 176 | 4-I | 0 | C(O)O | H | H | H | i-Propyl | oil |
| 177 | 4-I | 0 | C(O)O | H | H | H | CF(CH₃)₂ | |
| 178 | 4-Me | 0 | C(O)O | H | H | Me | i-Propyl | oil |
| 179 | 4-Me | 0 | C(O)O | H | H | Et | i-Propyl | oil |
| 180 | 4-Me | 0 | C(O)O | H | H | H | i-Propyl | oil |
| 181 | 4-Me | 0 | C(O)O | H | H | i-Propyl | i-Propyl | |
| 182 | 4-Me | 1 | O | H | H | Me | i-Propyl | resin |
| 183 | 4-Me | 1 | O | H | H | Me | CF(CH₃)₂ | resin |
| 184 | 4-n-Bu | 0 | C(O)O | H | H | Me | i-Propyl | oil |
| 185 | 4-n-Bu | 0 | C(O)O | H | H | Et | i-Propyl | oil |
| 186 | 4-n-Bu | 0 | C(O)O | H | H | H | i-Propyl | oil |
| 187 | 4-n-Bu | 2 | C(O)O | H | H | H | CF(CH₃)₂ | |
| 188 | 4-OCF₃ | 0 | C(O)O | H | H | Me | i-Propyl | oil |
| 189 | 4-OCF₃ | 0 | C(O)O | H | H | Et | i-Propyl | oil |
| 190 | 4-OCF₃ | 0 | C(O)O | H | H | H | i-Propyl | oil |
| 191 | 4-OCF₃ | 0 | C(O)O | H | H | Me | CF(CH₃)₂ | |
| 192 | 4-t-Bu | 0 | C(O)O | H | H | Me | i-Propyl | oil |
| 193 | 4-t-Bu | 0 | C(O)O | H | H | Et | i-Propyl | oil |
| 194 | 4-t-Bu | 0 | C(O)O | H | H | H | i-Propyl | oil |
| 195 | 4-t-Bu | 0 | C(O)O | Me | H | s-Butyl | CF(CH₃)₂ | |
| 196 | H | 0 | C(O)O | H | H | Me | i-Propyl | oil |
| 197 | H | 0 | C(O)O | H | H | Et | i-Propyl | oil |
| 198 | H | 0 | C(O)O | H | H | H | i-Propyl | oil |
| 199 | H | 0 | C(O)O | H | H | Me | CF(CH₃)₂ | |
| 200 | H | 1 | O | H | H | Me | i-Propyl | m.p. 104° C. |
| 201 | H | 1 | O | H | H | Me | CF(CH₃)₂ | resin |
| 202 | H | 1 | O | H | H | Me | CF₂CHF₂ | m.p. 118° C. |
| 203 | H | 1 | O | Me | H | Me | CHFCH₃ | |
| 204 | H | 1 | SO₂ | Me | H | Et | CHFCH₃ | |
| 205 | H | 1 | O | Me | H | c-Butyl | CF(CH₃)₂ | |
| 206 | H | 1 | O | Me | Me | H | CF(CH₃)₂ | |

TABLE 2
Compounds of the formula (Ib) (=formula I where n = 2, R¹, R², R³, R⁵,
R⁷, R⁹, R¹⁰ are each hydrogen and aryl = phenyl which is
substituted as shown in the table)

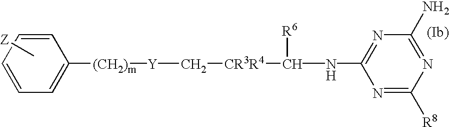

| No. | Z | m | Y | R³ | R⁴ | R⁶ | R⁸ | Phys. data |
|---|---|---|---|---|---|---|---|---|
| 207 | 3,5-F₂ | 0 | O | H | H | Me | CF(CH₃)2 | resin |
| 208 | 3,5-(OMe)₂ | 0 | O | H | H | t-Butyl | i-Propyl | |
| 209 | 3-Cl | 0 | O | H | H | Me | i-Propyl | resin |
| 210 | 3-F | 0 | O | Et | Et | H | CF₂CHF₂ | |
| 211 | 3-Me | 0 | O | H | H | c-Propyl | CF₂CHF₂ | |
| 212 | 3-Me,4-F | 0 | O | H | H | Me | CF(CH₃)₂ | resin |
| 213 | 3-OMe | 0 | O | H | H | Me | i-Propyl | resin |
| 214 | H | 0 | O | Me | Me | H | i-Propyl | oil |
| 215 | 3,4,5-Cl₃ | 1 | O | H | H | c-Pentyl | CHFCH₃ | |
| 216 | 3,5-F₂ | 0 | O | H | H | Et | CF₂CHF₂ | resin |
| 217 | 3,5-Me₂ | 0 | O | H | H | Et | CF(CH₃)₂ | resin |
| 218 | 3-CF₃ | 1 | C(O)O | H | H | Me | CHFCH₃ | |
| 219 | 3-Cl | 0 | O | H | H | c-Propyl | CF₂CHF₂ | |
| 220 | 3-Cl | 0 | O | H | H | Et | CHFCH₃ | resin |
| 221 | 3-Cl,5-Me | 1 | O | H | H | CH₂-cButyl | i-Propyl | |
| 222 | 3-F | 0 | O | H | H | Et | CF(CH₃)₂ | resin |
| 223 | 3-F | 0 | O | Et | H | H | CHFCH₃ | |
| 224 | 3-Me | 0 | O | Me | Me | H | i-Propyl | |
| 225 | 3-Me | 0 | O | Me | H | Me | CF(CH₃)₂ | resin |
| 226 | 3-Me,4-F | 0 | O | H | H | Et | CHFCH₃ | resin |
| 227 | 3-Me,4-F | 0 | O | H | H | c-Propyl | CF₂CHF₂ | resin |
| 228 | 3-OMe | 0 | O | H | H | Et | CF₂CHF₂ | resin |
| 229 | 3-OMe,5-Cl | 1 | O | H | H | n-Butyl | CF₂CHF₂ | |
| 230 | H | 0 | O | Me | H | Me | CF(CH₃)₂ | |
| 231 | H | 0 | O | H | H | Et | i-Propyl | resin |
| 232 | 3,5-F₂ | 0 | O | H | H | c-Propyl | CF₂CHF₂ | |
| 233 | 3,5-(OEt)₂ | 1 | S | H | H | CH₂OMe | CHFCH₃ | |
| 234 | 3-Cl | 0 | O | Me | H | Me | CHFCH₃ | resin |
| 235 | 3-F | 0 | O | Et | Et | H | CF(CH₃)₂ | |
| 236 | 3-Me | 0 | O | H | H | c-Propyl | CF(CH₃)₂ | resin |
| 237 | 3-Me | 0 | SO₂ | H | H | i-Propyl | i-Propyl | |
| 238 | 3-OMe | 0 | O | H | H | c-Butyl | CHFCH₃ | |
| 239 | H | 0 | O | H | H | c-Butyl | CHFCH₃ | resin |
| 240 | 3,5-F₂ | 0 | O | H | H | c-Propyl | CHFCH₃ | |
| 241 | 3,5-(OEt)₂ | 0 | O | H | H | CH₂-cButyl | CF₂CHF₂ | |
| 242 | 3-Cl | 0 | O | Me | H | Me | CF(CH₃)₂ | resin |
| 243 | 3-F | 0 | O | Et | Et | H | i-Propyl | |
| 244 | 3-Me | 0 | O | H | H | c-Propyl | i-Propyl | resin |
| 245 | 3-Me | 0 | O | H | H | Et | CF(CH₃)₂ | resin |
| 246 | 3-OMe | 0 | O | H | H | c-Butyl | CF(CH₃)₂ | |
| 247 | H | 0 | O | H | H | c-Butyl | CF(CH₃)₂ | |
| 248 | 3,5-Cl₂ | 0 | O | H | Et | i-Propyl | i-Propyl | |
| 249 | 3,5-(OEt)₂ | 1 | O | H | H | n-Butyl | CF₂CHF₂ | |
| 250 | 3-Cl | 0 | O | Me | Me | H | CHFCH₃ | |
| 251 | 3-F | 0 | O | H | H | c-Butyl | CF(CH₃)₂ | |
| 252 | 3-F,5-OMe | 1 | O | H | H | s-Butyl | i-Propyl | |
| 253 | 3-Me | 0 | O | H | H | Me | CF(CH₃)₂ | resin |
| 254 | 3-OEt | 1 | C(O)O | H | H | CH₂-cButyl | i-Propyl | |
| 255 | H | 0 | O | H | H | c-Propyl | CHFCH₃ | resin |
| 256 | 3,4,5-Cl₃ | 0 | O | H | H | CF₃ | CHFCH₃ | |
| 257 | 3,5-Cl₂ | 0 | O | H | H | CH₂OEt | CF(CH₃)₂ | |
| 258 | 3,5-Cl₂ | 1 | SO₂ | H | H | Me | CF₃ | |
| 259 | 3,5-Me₂ | 0 | O | H | H | c-Pentyl | CHFCH₃ | |
| 260 | 3,5-(OEt)₂ | 0 | O | H | H | Me | CHFCF₃ | |
| 261 | 3,5-(OEt)₂ | 0 | C(O)O | H | H | i-Propyl | i-Propyl | |
| 262 | 3-Cl | 0 | O | H | H | c-Butyl | CHFCH₃ | |
| 263 | 3-Cl | 0 | O | Me | Me | H | i-Propyl | resin |
| 264 | 3-Cl | 0 | O | Me | Me | H | CF(CH₃)₂ | resin |
| 265 | 3-F | 0 | O | H | H | c-Propyl | CF(CH₃)₂ | |
| 266 | 3-F | 0 | O | H | H | c-Propyl | CF₂CHF₂ | |
| 267 | 3-F | 0 | O | H | H | c-Butyl | i-Propyl | |
| 268 | 3-F,5-Me | 0 | SO2 | H | H | Me | CF(CH₃)₂ | |
| 269 | 3-F,5-Me | 0 | O | H | H | n-Butyl | CF₂CHF₂ | |
| 270 | 3-F,5-OMe | 0 | C(O)O | H | H | c-Pentyl | CF₃ | |
| 271 | 3-Me | 0 | O | Me | H | H | i-Propyl | resin |
| 272 | 3-Me | 0 | O | Me | H | H | CF₂CHF₂ | resin |
| 273 | 3-Me | 0 | O | H | H | Me | i-Propyl | resin |
| 274 | 3-Me,4-F | 0 | O | H | H | c-Butyl | CHFCH₃ | resin |
| 275 | 3-OEt | 2 | O | H | H | CH₂F | i-Propyl | |
| 276 | 3-aEt | 0 | O | H | H | CH₂-c-Propyl | CF₃ | |
| 277 | 3-Ph | 1 | SO₂ | H | Me | t-Butyl | i-Propyl | |
| 278 | H | 0 | O | H | H | c-Propyl | i-Propyl | resin |
| 279 | H | 0 | O | H | H | c-Propyl | CF(CH₃)₂ | resin |
| 280 | H | 0 | O | H | H | Me | CF₃ | |
| 281 | 3,5-F₂ | 0 | O | H | H | Me | i-Propyl | resin |
| 282 | 3,5-Me₂ | 0 | O | H | H | Me | CF(CH₃)₂ | m.p. 131° C. |
| 283 | 3,5-(OMe)₂ | 0 | C(O)O | H | H | s-Butyl | CHFCF₃ | |
| 284 | 3-Cl | 0 | O | H | H | c-Propyl | i-Propyl | resin |
| 285 | 3-Cl | 0 | O | Me | H | Me | CF₂CHF₂ | resin |
| 286 | 3-Cl | 0 | O | H | H | CH₂-c-Propyl | CF(CH₃)₂ | |
| 287 | 3-F | 0 | O | Et | Et | H | CHFCH₃ | |
| 288 | 3-F | 0 | O | H | H | CH₂-c-Butyl | CF₃ | |
| 289 | 3-Me | 0 | O | H | H | c-Propyl | CHFCH₃ | resin |
| 290 | 3-Me | 0 | O | Me | Me | H | CHFCH₃ | |
| 291 | 3-Me,4-F | 0 | O | H | H | Me | i-Propyl | resin |
| 292 | 3-Me,4-F | 0 | O | H | H | c-Propyl | i-Propyl | resin |
| 293 | 3-OMe | 0 | O | H | H | c-Butyl | CF₂CHF₂ | |
| 294 | 3-OMe,5-Cl | 0 | O | H | H | Me | i-Propyl | resin |
| 295 | H | 0 | O | H | H | c-Butyl | CF₂CHF₂ | |
| 296 | H | 0 | O | Me | H | Me | CF₂CHF₂ | |
| 297 | 3,5-F₂ | 0 | O | H | H | Me | CF₂CHF₂ | resin |
| 298 | 3,5-(OMe)₂ | 0 | O | H | Me | CF₃ | CF₂CHF₂ | |
| 299 | 3-Cl | 0 | O | H | H | Me | CF(CH₃)₂ | resin |
| 300 | 3-F | 0 | O | H | H | Me | i-Propyl | resin |
| 301 | 3-Me | 0 | O | H | H | c-Butyl | i-Propyl | resin |
| 302 | 3-Me,4-F | 0 | O | H | H | Me | CHFCH₃ | |
| 303 | 3-OMe | 0 | O | H | H | Me | CF(CH₃)₂ | resin |
| 304 | H | 0 | O | Me | Me | H | CF(CH₃)₂ | resin |
| 305 | 3,5-F₂ | 0 | O | H | H | Et | i-Propyl | resin |
| 306 | 3,5-(OMe)₂ | 0 | O | H | H | CH₂F | CHFCH₃ | |
| 307 | 3-Cl | 0 | O | H | H | Me | CF₂CHF₂ | resin |
| 308 | 3-F | 0 | O | H | H | Me | CF(CH₃)₂ | resin |
| 309 | 3-Me | 0 | O | H | H | c-Butyl | CF(CH₃)₂ | resin |
| 310 | 3-Me,4-F | 0 | O | H | H | Me | CF₂CHF₂ | resin |
| 311 | 3-OMe | 0 | O | H | H | Me | CF₂CHF₂ | resin |
| 312 | H | 0 | O | Me | Me | H | CHFCH₃ | resin |
| 313 | 3,5-Cl₂ | 0 | SO2 | H | H | CH₂OMe | CHFCH₃ | |
| 314 | 3,5-(OEt)₂ | 0 | O | H | H | CH₂OEt | CHFCH₃ | |
| 315 | 3-Cl | 0 | O | H | H | c-Butyl | CF₂CHF₂ | |
| 316 | 3-F | 0 | O | H | H | c-Propyl | CHFCH₃ | |
| 317 | 3-F.5-Me | 0 | O | H | H | i-Propyl | i-Propyl | |
| 318 | 3-Me | 0 | O | Me | H | H | CF(CH₃)₂ | resin |
| 319 | 3-Me,4-F | 0 | O | H | H | c-Butyl | CF₂CHF₂ | |
| 320 | 3-Ph | 0 | O | H | H | CF₃ | CF₂CHF₂ | |
| 321 | 3,5-F₂ | 0 | O | H | H | c-Propyl | i-Propyl | |
| 322 | 3,5-(OEt)₂ | 0 | S | H | H | CH₂F | CHFCF₃ | |
| 323 | 3-Cl | 0 | O | Me | Me | H | CF₂CHF₂ | |
| 324 | 3-F | 0 | O | H | H | c-Butyl | CHFCH₃ | |
| 325 | 3-F,5-OMe | 0 | O | H | H | 1-Butyl | CF₂CHF₂ | |
| 326 | 3-Me | 0 | O | H | H | Me | CF₂CHF₂ | resin |
| 327 | 3-OEt | 1 | 0 | H | Et | CH₂OMe | CF₂CHF₂ | |
| 328 | H | 0 | O | H | H | c-Propyl | CF₂CHF₂ | |
| 329 | 3,5-Me₂ | 0 | O | H | H | Me | CF₂CHF₂ | resin |
| 330 | 3-Cl | 0 | O | H | H | c-Propyl | CF(CH₃)₂ | resin |
| 331 | 3-Cl,5-Me | 1 | C(O)O | H | Me | CH₂F | CF(CH₃)₂ | |
| 332 | 3-F | 0 | O | H | H | H | i-Propyl | |
| 333 | 3-Me | 0 | O | Me | Me | H | CF₂CHF₂ | |
| 334 | 3-Me,4-F | 0 | O | H | H | c-Propyl | CF(CH₃)₂ | resin |
| 335 | 3-OMe,5-Cl | 0 | O | H | H | Me | CF(CH₃)₂ | resin |
| 336 | H | 0 | O | H | H | Me | i-Propyl | resin |
| 337 | 3,4,5-Cl₃ | 0 | S | H | H | s-Butyl | CF(CH₃)₂ | |
| 338 | 3,5-F₂ | 0 | O | H | H | c-Propyl | CF(CH₃)₂ | |
| 339 | 3,5-Me₂ | 0 | O | H | H | Me | i-Propyl | resin |
| 340 | 3,5-Me₂ | 0 | O | H | H | Et | i-Propyl | resin |
| 341 | 3,5-Me₂ | 0 | O | H | H | Et | CHFCH₃ | resin |
| 342 | 3,5-(OEt)₂ | 1 | O | H | H | CH₂-c-Propyl | i-Propyl | |
| 343 | 3-CF₃ | 0 | S | H | H | Me | CF(CH₃)₂ | |
| 344 | 3-Cl | 0 | O | H | H | c-Propyl | CHFCH₃ | resin |
| 345 | 3-Cl | 0 | O | H | H | c-Butyl | i-Propyl | |
| 346 | 3-Cl | 0 | O | Me | H | Me | i-Propyl | resin |
| 347 | 3-Cl | 0 | O | H | H | Et | CF₂CHF₂ | resin |
| 348 | 3-Cl,5-Me | 0 | O | H | Et | CH₂-c-Propyl | CF₃ | |
| 349 | 3-Cl,5-Me | 0 | O | H | H | CH₂OMe | CF₂CHF₂ | |
| 350 | 3-F | 0 | O | H | H | c-Butyl | CF₂CHF₂ | |
| 351 | 3-F | 0 | O | H | H | Et | CHFCH₃ | resin |
| 352 | 3-F | 2 | 0 | H | H | H | CF(CH₃)₂ | |
| 353 | 3-F | 0 | O | H | H | H | CF₂CHF₂ | |
| 354 | 3-F,5-OMe | 0 | O | H | H | CF₃ | CHFCH₃ | |
| 355 | 3-Me | 0 | O | Me | Me | H | CF(CH₃)₂ | |
| 356 | 3-Me | 0 | O | Me | H | Me | i-Propyl | oil |
| 357 | 3-Me | 0 | O | Me | H | Me | CHFCH₃ | oil |
| 358 | 3-Me | 0 | O | H | H | Et | i-Propyl | resin |
| 359 | 3-Me,4-F | 0 | O | H | H | Et | CF₂CHF₂ | |
| 360 | 3-Me,4-F | 0 | O | H | H | c-Propyl | CHFCH₃ | resin |
| 361 | 3-Me,4-F | 0 | O | H | H | c-Butyl | i-Propyl | |
| 362 | 3-OMe | 0 | O | H | H | c-Butyl | i-Propyl | |
| 363 | 3-OMe | 0 | O | H | Me | n-Butyl | CF₂CHF₂ | |
| 364 | 3-OMe,5-Cl | 0 | O | H | H | Me | CF₂CHF₂ | resin |
| 365 | 3-Ph | 1 | 0 | H | H | c-Pentyl | CHFCH₃ | |
| 366 | H | 0 | O | H | H | c-Butyl | i-Propyl | resin |
| 367 | H | 0 | O | Me | H | Me | CHFCH₃ | |
| 368 | H | 0 | O | H | H | Me | CF(CH₃)₂ | resin |
| 369 | H | 0 | O | H | H | Et | CF(CH₃)₂ | resin |
| 370 | 3,5-F₂ | 0 | O | H | H | Et | CF(CH₃)₂ | resin |
| 371 | 3-CF₃ | 0 | O | H | H | CH₂OEt | CF(CH₃)₂ | |
| 372 | 3-Cl | 0 | O | H | H | Et | i-Propyl | resin |
| 373 | 3-F | 0 | O | H | H | Me | CF₂CHF₂ | resin |
| 374 | 3-Me | 0 | O | H | H | c-Butyl | CHFCH₃ | resin |
| 375 | 3-Me,4-F | 0 | O | H | H | Et | i-Propyl | resin |
| 376 | 3-OMe | 0 | O | H | H | Et | i-Propyl | resin |
| 377 | H | 0 | O | Me | Me | H | CF₂CHF₂ | |
| 378 | 3,5-F₂ | 0 | O | H | H | Et | CHFCH₃ | resin |
| 379 | 3-CF₃ | 0 | O | H | H | Me | CF₃ | |
| 380 | 3-Cl | 0 | O | H | H | Et | CF(CH₃)₂ | resin |
| 381 | 3-F | 0 | O | H | H | Et | i-Propyl | resin |
| 382 | 3-Me | 0 | O | H | H | c-Butyl | CF₂CHF₂ | |
| 383 | 3-Me,4-F | 0 | O | H | H | Et | CF(CH₃)₂ | resin |
| 384 | 3-OMe | 0 | O | H | H | Et | CF(CH₃)₂ | resin |
| 385 | H | 0 | O | Me H | Me | i-Propyl | | |
| 386 | 3,4,5-Cl₃ | 0 | O | H | H | t-Butyl | CF3 | |
| 387 | 3,5-Me₂ | 0 | O | H | H | Et | CF₂CHF₂ | resin |
| 388 | 3-Cl | 0 | O | H | H | c-Butyl | CF(CH₃)₂ | resin |
| 389 | 3-F | 0 | O | H | H | c-Propyl | i-Propyl | |
| 390 | 3-F,5-Me | 2 | O | H | H | CH₂OEt | CHFCH₃ | |
| 391 | 3-Me | 0 | O | Me | H | Me | CF₂CHF₂ | |
| 392 | 3-Me,4-F | 0 | O | H | H | c-Butyl | CF(CH₃)₂ | |
| 393 | 3-Ph | 0 | SO₂ | H | H | 5-Butyl | CF(CH₃)₂ | |
| 394 | H | 0 | O | H | H | Et | CF₂CHF₂ | resin |
| 395 | 4-F | 0 | O | H | H | c-Butyl | i-Propyl | resin |
| 396 | 4-F | 0 | O | H | H | c-Butyl | CF(CH₃)₂ | resin |
| 397 | 4-F | 0 | O | H | H | c-Butyl | CHFCH₃ | resin |
| 398 | 4-F | 0 | O | H | H | c-Butyl | CF₂CHF₂ | resin |
| 399 | 3,4-F₂ | 0 | O | H | H | c-Butyl | i-Propyl | resin |
| 400 | 3,4-F₂ | 0 | O | H | H | c-Butyl | CF(CH₃)₂ | resin |
| 401 | 3,4-F₂ | 0 | O | H | H | c-Butyl | CHFCH₃ | resin |
| 402 | 3,4-F₂ | 0 | O | H | H | c-Butyl | CF₂CHF₂ | resin |
| 403 | H | 0 | O | H | H | c-Butyl | CCl(CH₃)₂ | resin |
| 404 | 3-Me | 0 | O | H | H | c-Butyl | CCl(CH₃)₃ | resin |
| 405 | H | 0 | O | H | H | c-Butyl | Me | resin |
| 406 | 3-Me | 0 | O | H | H | c-Butyl | Et | resin |
| 407 | H | 0 | O | H | H | c-Butyl | Me | resin |
| 408 | 3-Me | 0 | O | H | H | c-Butyl | Et | resin |
| 409 | 3-Me | 0 | O | H | H | c-Butyl | CF₂CF₃ | resin |
| 410 | 4-F | 0 | O | H | H | c-Propyl | i-Propyl | resin |
| 411 | 4-F | 0 | O | H | H | c-Propyl | CF(CH₃)₂ | resin |
| 412 | 4-F | 0 | O | H | H | c-Propyl | CHFCH₃ | resin |
| 413 | 4-F | 0 | O | H | H | c-Propyl | CF₂CHF₂ | resin |
| 414 | 3,4-F₂ | 0 | O | H | H | c-Propyl | i-Propyl | |
| 415 | 3,4-F₂ | 0 | O | H | H | c-Propyl | CF(CH₃)₂ | |
| 416 | 3,4-F₂ | 0 | O | H | H | c-Propyl | CHFCH₃ | |
| 417 | 3,4-F₂ | 0 | O | H | H | c-Propyl | CF₂CHF₂ | |
| 418 | H | 0 | O | H | H | c-Propyl | CCl(CH₃)₂ | resin |
| 419 | 3-Me | 0 | O | H | H | c-Propyl | CCl(CH₃)₃ | resin |
| 420 | H | 0 | O | H | H | c-Propyl | Me | resin |
| 421 | 3-Me | 0 | O | H | H | c-Propyl | Et | resin |
| 422 | H | 0 | O | H | H | c-Propyl | Me | resin |
| 423 | 3-Me | 0 | O | H | H | c-Propyl | Et | resin |
| 424 | 3-Me | 0 | O | H | H | c-Propyl | CF₂CF₃ | resin |
| 425 | 3-Me | 0 | O | H | H | Et | Me | resin |
| 426 | 3-Me | 0 | O | H | H | Et | Et | resin |
| 427 | 3-Me | 0 | O | H | H | Et | CF₂CF₃ | resin |
| 428 | 4-F | 1 | O | H | H | Et | CF(CH₃)₂ | |
| 429 | 4-F | 1 | O | H | H | Et | CHFCH₃ | |
| 430 | 3-Cl | 1 | O | H | H | Et | CF(CH₃)₂ | |
| 431 | 3-Cl | 1 | O | H | H | Et | CHFCH₃ | |
| 432 | 3-Me | 1 | O | H | H | Et | CF(CH₃)₂ | |
| 433 | 3-Me | 1 | O | H | H | Et | CHFCH₃ | |
| 434 | 3-Me,4-F | 1 | O | H | H | Et | CF(CH₃)₂ | |
| 435 | 3-Me,4-F | 1 | O | H | H | Et | CHFCH₃ | |
| 436 | 3,5-Me₂ | 1 | O | H | H | Et | CF(CH₃)₂ | |
| 437 | 3,5-Me₂ | 1 | O | H | H | Et | CHFCH₃ | |
| 438 | 3-F | 1 | O | H | H | Et | CF(CH₃)₂ | |
| 439 | 3-F | 1 | O | H | H | Et | CHFCH₃ | |
| 440 | H | 1 | O | H | H | Et | CF(CH₃)₂ | |
| 441 | H | 1 | O | H | H | Et | CHFCH₃ | |
| 442 | 3-Cl,5-F | 1 | O | H | H | Et | CF(CH₃)₂ | |
| 443 | 3-Cl,5-F | 1 | O | H | H | Et | CHFCH₃ | |
| 444 | 3-Cl | 2 | O | H | H | Et | CF(CH₃)₂ | |
| 445 | 3-Cl | 2 | O | H | H | Et | CHFCH₃ | |
| 446 | 3-Me | 2 | O | H | H | Et | CF(CH₃)₂ | |
| 447 | 3-Me | 2 | O | H | H | Et | CHFCH₃ | |
| 448 | 3-Me,4-F | 2 | O | H | H | Et | CF(CH₃)₂ | |
| 449 | 3-Me,4-F | 2 | O | H | H | Et | CHFCH₃ | |
| 450 | 3,5-Me₂ | 2 | O | H | H | Et | CF(CH₃)₂ | |
| 451 | 3,5-Me₂ | 2 | O | H | H | Et | CHFCH₃ | |
| 452 | 3-F | 2 | O | H | H | Et | CF(CH₃)₂ | |
| 453 | 3-F | 2 | O | H | H | Et | CHFCH₃ | |
| 454 | H | 2 | O | H | H | Et | CF(CH₃)₂ | |
| 455 | H | 2 | O | H | H | Et | CHFCH₃ | |
| 456 | 3-Cl | 0 | O | Et | Et | H | CF(CH₃)₂ | resin |
| 457 | 3-Cl | 0 | O | Et | Et | H | CHFCH₃ | resin |
| 458 | 3-Me | 0 | O | H | H | Me | Me | resin |
| 459 | 3-Me | 0 | O | H | H | Me | Et | resin |
| 460 | 3-Me | 0 | O | H | H | Me | CF₂CF₃ | resin |
| 461 | 4-F | 1 | O | H | H | Me | CF(CH₃)₂ | |
| 462 | 4-F | 1 | O | H | H | Me | CHFCH₃ | |
| 463 | 3-Cl | 1 | O | H | H | Me | CF(CH₃)₂ | |
| 464 | 3-Ci | 1 | O | H | H | Me | CHFCH₃ | |
| 465 | 3-Me | 1 | O | H | H | Me | CF(CH₃)₂ | |
| 466 | 3-Me | 1 | O | H | H | Me | CHFCH₃ | |
| 467 | 3-Me,4-F | 1 | O | H | H | Me | CF(CH₃)₂ | |
| 468 | 3-Me,4-F | 1 | O | H | H | Me | CHFCH₃ | |
| 469 | 3,5-Me₂ | 1 | O | H | H | Me | CF(CH₃)₂ | |
| 470 | 3,5-Me₂ | 1 | O | H | H | Me | CHFCH₃ | |
| 471 | 3-F | 1 | O | H | H | Me | CF(CH₃)₂ | |
| 472 | 3-F | 1 | O | H | H | Me | CHFCH₃ | |
| 473 | H | 1 | O | H | H | Me | CF(CH₃)₂ | |
| 474 | H | 1 | O | H | H | Me | CHFCH₃ | |
| 475 | 3-Cl,5-F | 1 | O | H | H | Me | CF(CH₃)₂ | |
| 476 | 3-Cl,5-F | 1 | O | H | H | Me | CHFCH₃ | |
| 477 | 3-Cl | 2 | O | H | H | Me | CF(CH₃)₂ | |
| 478 | 3-Cl | 2 | O | H | H | Me | CHFCH₃ | |
| 479 | 3-Me | 2 | O | H | H | Me | CF(CH₃)₂ | |
| 480 | 3-Me | 2 | O | H | H | Me | CHFCH₃ | |
| 481 | 3-Me,4-F | 2 | O | H | H | Me | CF(CH₃)₂ | |
| 482 | 3-Me,4-F | 2 | O | H | H | Me | CHFCH₃ | |
| 483 | 3,5-Me₂ | 2 | O | H | H | Me | CF(CH₃)₂ | |
| 484 | 3,5-Me₂ | 2 | O | H | H | Me | CHFCH₃ | |
| 485 | 3-F | 2 | O | H | H | Me | CF(CH₃)₂ | |
| 486 | 3-F | 2 | O | H | H | Me | CHFCH₃ | |
| 487 | H | 2 | O | H | H | Me | CF(CH₃)₂ | |
| 488 | H | 2 | O | H | H | Me | CHFCH₃ | |

TABLE 3
Compounds of the formula (Ic) (=formula I where n = 3;
R¹, R², R⁵, R⁷, R⁹, R¹⁰ are each hydrogen and
aryl = phenyl which is substituted as shown in the table)

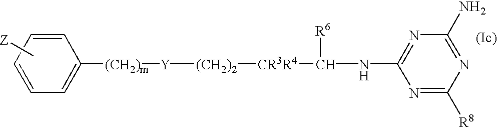

| No. | Z | m | Y | R³ | R⁴ | R⁶ | R⁸ | Phys. data |
|---|---|---|---|---|---|---|---|---|
| 489 | 3-F | 0 | O | H | H | Me | CHFCH₃ | |
| 490 | H | 0 | O | H | H | Et | CF(CH₃)₂ | resin |
| 491 | 3-Me | 0 | O | H | Me | c-Propyl | i-Propyl | |
| 492 | 3-Cl | 0 | O | H | H | c-Propyl | CF₂CHF₂ | |
| 493 | 3-F | 0 | O | H | H | c-Butyl | CHFCH₃ | |
| 494 | 3,5-Me₂ | 0 | O | H | H | t-Butyl | i-Propyl | |
| 495 | 3-Cl,5-Me | 2 | SO₂ | H | H | Me | CHFCF₃ | |
| 496 | 3-F,5-Me | 0 | O | H | H | CH₂F | CF(CH₃)₂ | |
| 497 | H | 0 | O | H | H | Et | CF₂CHF₂ | |
| 498 | 3-F | 0 | O | H | H | c-Propyl | CF(CH₃)₂ | |
| 499 | 3,5-Me₂ | 0 | O | H | H | CH₂F | CHFCH₃ | |
| 500 | 3-F,5-OMe | 0 | O | H | H | CH₂-c-Butyl | i-Propyl | |
| 501 | H | 0 | O | H | H | Me | CF(CH₃)₂ | resin |
| 502 | 3-Me | 0 | O | H | H | Me | CF(CH₃)₂ | resin |
| 503 | 3-Me | 0 | O | H | H | Et | CF₂CHF₂ | |
| 504 | 3-Cl | 0 | O | H | H | Et | CF₂CHF₂ | |
| 505 | H | 0 | O | H | H | c-Butyl | CF(CH₃)₂ | |
| 506 | 3-Me | 0 | O | H | H | c-Butyl | CF(CH₃)₂ | |
| 507 | 3,5-(OMe)₂ | 1 | S | H | Et | H | CF(CH₃)₂ | |
| 508 | 3,5-F₂ | 0 | O | H | H | c-Pentyl | CHFCH₃ | |
| 509 | 3-CF₃ | 2 | S | H | H | i-Propyl | i-Propyl | |
| 510 | H | 0 | O | H | H | Et | i-Propyl | resin |
| 511 | 3-Cl | 0 | O | H | H | H | CHFCH₃ | |
| 512 | 3,5-Me₂ | 0 | O | H | H | s-Butyl | CHFCF₃ | |
| 513 | 3-F,5-Me | 1 | 0 | H | H | CF₃ | CHFCH₃ | |
| 514 | 3-Me | 0 | O | H | H | Et | CF(CH₃)₂ | resin |
| 515 | 3-F | 0 | O | H | H | c-Propyl | CF₂CHF₂ | |
| 516 | 3,5-(OMe)₂ | 1 | C(O)O | H | Et | CH₂-c-Butyl | CF₃ | |
| 517 | 3-F,5-OMe | 0 | O | H | H | CH₂OEt | CHFCH₃ | |
| 518 | H | 0 | O | H | H | Me | CHFCH₃ | resin |
| 519 | 3-Me | 0 | O | H | H | Me | CF₂CHF₂ | |
| 520 | 3-Cl | 0 | O | H | H | Et | i-Propyl | |
| 521 | 3-F | 0 | O | H | H | Et | CF(CH₃)₂ | |
| 522 | H | 0 | O | H | H | c-Butyl | CHFCH₃ | |
| 523 | 3-Me | 0 | O | H | H | H | CF₂CHF₂ | |
| 524 | 3,5-F₂ | 1 | 0 | H | Me | Me | CF₃ | |
| 525 | 3-OMe,5-Cl | 0 | O | H | H | t-Butyl | i-Propyl | |
| 526 | 3-CF₃ | 0 | O | H | H | n-ButyI | CF₂CHF₂ | |
| 527 | 3-Cl | 0 | O | H | H | Me | CF₂CHF₂ | |
| 528 | H | 0 | O | H | H | c-Propyl | CF(CH₃)₂ | resin |
| 529 | 3-Cl | 0 | O | H | H | c-Butyl | CF₂CHF₂ | |
| 530 | 3-OEt | 1 | O | H | H | CH₂-c-Butyl | i-Propyl | |
| 531 | H | 0 | O | H | H | Me | i-Propyl | resin |
| 532 | 3-Me | 0 | O | H | H | Me | CHFCH₃ | resin |
| 533 | 3-F | 0 | O | H | H | H | CF(CH₃)₂ | |
| 534 | 3-Me,4-F | 0 | O | H | H | Me | CF₂CHF₂ | |
| 535 | 3-Me | 0 | O | H | H | Et | CHFCH₃ | resin |
| 536 | 3-F | 0 | O | H | H | Et | i-Propyl | |
| 537 | H | 0 | O | H | H | c-Propyl | CF₂CHF₂ | |
| 538 | 3-Cl | 0 | O | H | H | c-Propyl | CF(CH₃)₂ | |
| 539 | H | 0 | O | H | H | c-Butyl | i-Propyl | |
| 540 | 3-Me | 0 | O | H | H | c-Butyl | CHFCH₃ | |
| 541 | 3-F | 0 | O | H | H | c-Butyl | CF(CH₃)₂ | |
| 542 | 3-OMe | 0 | S | H | H | c-Pentyl | CHFCH₃ | |
| 543 | 3,5-(OMe)₂ | 0 | O | H | H | CH₂OMe | CHFCH₃ | |
| 544 | 3-OMe,5-Cl | 0 | S | H | H | s-Butyl | CF(CH₃)₂ | |
| 545 | 3-OEt | 0 | O | H | H | CH_{2pk OEt} | CHFCH₃ | |
| 546 | 3-F,5-Me | 0 | S | H | H | t-Butyl | CF₃ | |
| 547 | 3-CF₃ | 0 | O | H | H | Me | CF(CH₃)₂ | |
| 548 | 3-Me,4-F | 0 | O | H | H | Me | CF(CH₃)₂ | |
| 549 | 3-Me | 0 | O | H | H | c-Propyl | CF₂CHF₂ | |
| 550 | 3-OMe | 0 | C(O)O | H | H | i-Propyl | i-Propyl | |
| 551 | 3-Cl,5-Me | 0 | O | H | H | c-Pentyl | CHFCH₃ | |
| 552 | 3-Cl | 0 | O | H | H | Me | CHFCH₃ | |
| 553 | 3-Me | 0 | O | H | H | Et | i-Propyl | resin |
| 554 | H | 0 | O | H | H | c-Propyl | i-Propyl | resin |
| 555 | 3-F | 0 | O | H | H | c-Propyl | CHFCH₃ | |
| 556 | 3-Cl | 0 | O | H | Me | c-Butyl | CHFCH₃ | |
| 557 | 3,5-(OMe)₂ | 0 | O | H | H | CH₂-c-Propyl | CF(CH₃)₂ | |
| 558 | 3-OEt | 0 | O | H | H | CH₂-c-Propyl | CF₃ | |
| 559 | 3-F,5-OMe | 0 | SO₂ | H | H | CH₂OMe | CF₂CHF₂ | |
| 560 | 3-Cl | 0 | O | H | H | Me | CF(CH₃)₂ | |
| 561 | 3-F | 0 | O | H | Et | H | CF₂CHF₂ | |
| 562 | 3-Cl | 0 | O | H | H | c-Butyl | CF(CH₃)₂ | |
| 563 | 3-OMe,5-Cl | 0 | O | H | H | CH₂F | i-Propyl | |
| 564 | 3-Cl | 0 | O | H | H | Me | i-Propyl | |
| 565 | 3-F | 0 | O | H | H | Et | CHFCH₃ | |
| 566 | 3-Cl | 0 | O | H | H | c-Butyl | i-Propyl | |
| 567 | 3-OMe,5-Cl | 0 | O | H | Me | CF₃ | CF₂CHF₂ | |
| 568 | H | 0 | O | H | H | Et | CHFCH₃ | resin |
| 569 | 3-F | 0 | O | H | H | c-Propyl | i-Propyl | |
| 570 | 3,5-Me₂ | 0 | O | H | H | CF₃ | CF₂CHF₂ | |
| 571 | 3-F,5-OMe | 0 | O | H | H | CH₂-c-Propyl | CF₃ | |
| 572 | 3-F | 0 | O | H | H | Me | i-Propyl | |
| 573 | H | 0 | O | H | H | c-Propyl | CHFCH₃ | resin |
| 574 | 3-F | 0 | O | H | H | H | i-Propyl | |
| 575 | 3-OEt | 0 | O | H | H | H | CF₂CHF₂ | |
| 576 | 3-Me,4-F | 0 | O | H | H | H | i-Propyl | |
| 577 | 3-Me | 0 | O | H | H | c-Propyl | CHFCH₃ | resin |
| 578 | 3-OMe | 0 | O | H | Me | Me | CF₃ | |
| 579 | 3-Cl,5-Me | 0 | O | H | H | n-Butyl | CF₂CHF₂ | |
| 580 | 3-F | 0 | O | H | H | Me | CF₂CHF₂ | |
| 581 | 3-Me,4-F | 0 | O | H | H | Me | CHFCH₃ | |
| 582 | 3-Me | 0 | O | H | H | c-Propyl | CF(CH₃)₂ | resin |
| 583 | 3-Cl | 0 | O | H | H | c-Propyl | i-Propyl | |
| 584 | 3-F | 0 | O | H | H | c-Butyl | CF₂CHF₂ | |
| 585 | 3-OMe | 1 | 0 | H | H | n-Butyl | CF₂CHF₂ | |
| 586 | 3-Cl,5-Me | 0 | O | H | H | i-Propyl | i-Propyl | |
| 587 | 3-F,5-Me | 0 | O | H | Et | H | CF(CH₃)₂ | |
| 588 | H | 0 | O | H | H | Me | CF₂CHF₂ | resin |
| 589 | 3-Cl | 0 | O | H | H | Et | CF(CH₃)₂ | |
| 590 | H | 0 | O | H | H | c-Butyl | CF₂CHF₂ | |
| 591 | 3,5-F₂ | 0 | S | H | H | i-Propyl | i-Propyl | |
| 592 | 3-CF₃ | 0 | SO₂ | H | H | c-Pentyl | CF₃ | |
| 593 | 3-Me | 0 | O | H | H | Me | i-Propyl | resin |
| 594 | 3-Cl | 0 | O | H | H | Et | CHFCH₃ | |
| 595 | 3-Me | 0 | O | H | H | c-Butyl | i-Propyl | resin |
| 596 | 3,5-F₂ | 0 | O | H | H | n-Butyl | CF₂CHF₂ | |

TABLE 4
Compounds of the formula (Id) (=formula I where n = 4
R¹, R², R⁵, R⁷, R⁹ and R¹⁰ are each hydrogen and
aryl phenyl which is substituted as shown in the table)

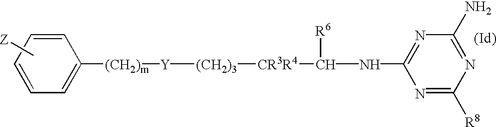

| | | | | | | | | Phys. |
|---|---|---|---|---|---|---|---|---|
| No. | Z | m | Y | R | R | R | R | data |
| 597 | 3-F | 0 | O | H | H | Me | CHFCH₃ | |
| 598 | H | 0 | O | H | H | Et | CF(CH₃)₂ | oil |
| 599 | 3-Me | 0 | O | H | Me | c-Propyl | 1-Propyl | |
| 600 | 3-Cl | 0 | O | H | H | c-Propyl | CF₂CHF₂ | |
| 601 | 3-F | 0 | O | H | H | c-Butyl | CHFCH₃ | |
| 602 | 3,5-Me₂ | 0 | O | H | H | t-Butyl | 1-Propyl | |
| 603 | 3-Cl,5-Me | 2 | SO₂ | H | H | Me | CHFCF₃ | |
| 604 | 3-F,5-Me | 0 | O | H | H | CH₂F | CF(CH₃)₂ | |
| 605 | H | 0 | O | H | H | Et | CF₂CHF₂ | oil |
| 606 | 3-F | 0 | O | H | H | c-Propyl | CF(CH₃)₂ | |
| 607 | 3,5-Me₂ | 0 | O | H | H | CH₂F | CHFCH₃ | |
| 608 | 3-F,5-OMe | 0 | O | H | H | CH₂-c-Butyl | i-Propyl | |
| 609 | H | 0 | O | H | H | Me | CF(CH₃)₂ | oil |
| 610 | 3-Me | 0 | O | H | H | Me | CF(CH₃)₂ | |
| 611 | 3-Me | 0 | O | H | H | Et | CF₂CHF₂ | |
| 612 | 3-Cl | 0 | O | H | H | Et | CF₂CHF₂ | |
| 613 | H | 0 | O | H | H | c-Butyl | CF(CH₃)₂ | |
| 613 | H | 0 | O | H | H | c-Butyl | CF(CH₃)₂ | |
| 614 | 3-Me | 0 | O | H | H | c-Butyl | CF(CH₃)₂ | |
| 615 | 3,5-(OMe)₂ | 1 | S | H | Et | H | CF(CH₃)₂ | |
| 616 | 3,5-F | 0 | O | H | H | c-Pentyl | CHFCH₃ | |
| 617 | 3-CF₃ | 2 | S | H | H | 1-Propyl | 1-Propyl | |
| 618 | H | 0 | O | H | H | Et | 1-Propyl | oil |
| 619 | 3-Cl | 0 | O | H | H | H | CHFCH₃ | |
| 620 | 3,5-Me₂ | 0 | O | H | H | s-Butyl | CHFCF₃ | |
| 621 | 3-F,5-Me | 1 | 0 | H | H | CF₃ | CHFCH₃ | |
| 622 | 3-Me | 0 | O | H | H | Et | CF(CH₃)₂ | |
| 623 | 3-F | 0 | O | H | H | c-Propyl | CF₂CHF₂ | |
| 624 | 3,5-(OMe)₂ | 1 | C(O)O | H | Et | CH₂-c-Butyl | CF₃ | |
| 625 | 3-F,5-OMe | 0 | 0 | H | H | CH₂OEt | CHFCH₃ | |
| 626 | H | 0 | O | H | H | Me | CHFCH₃ | oil |
| 627 | 3-Me | 0 | O | H | H | Me | CF₂CHF₂ | |
| 628 | 3-Cl | 0 | O | H | H | Et | i-Propyl | |
| 629 | 3-F | 0 | O | H | H | Et | CF(CH₃)₂ | |
| 630 | H | 0 | O | H | H | c-Butyl | CHFCH₃ | |
| 631 | 3-Me | 0 | O | H | H | H | CF₂CHF₂ | |
| 632 | 3,5-F₂ | 1 | 0 | H | Me | Me | CF₃ | |
| 633 | 3-OMe,5-Cl | 0 | 0 | H | H | t-Butyl | i-Propyl | |
| 634 | 3-CF₃ | 0 | O | H | H | n-Butyl | CF₂CHF₂ | |
| 635 | 3-Ct | 0 | O | H | H | Me | CF₂CHF₂ | |
| 636 | H | 0 | O | H | H | c-Propyl | CF(CH₃)₂ | oil |
| 637 | 3-Cl | 0 | O | H | H | c-Butyl | CF₂CHF₂ | |
| 638 | 3-OEt | 1 | 0 | H | H | CH₂-c-Butyl | i-Propyl | |
| 639 | H | 0 | O | H | H | Me | 1-Propyl | oil |
| 640 | 3-Me | 0 | O | H | H | Me | CHFCH₃ | |
| 641 | 3-F | 0 | O | H | H | H | CF(CH₃)₂ | |
| 642 | 3-Me,4-F | 0 | O | H | H | Me | CF₂CHF₂ | |
| 643 | 3-Me | 1 | 0 | H | H | Et | CHFCH₃ | |
| 644 | 3-F | 0 | O | H | H | Et | 1-Propyl | |
| 645 | H | 0 | O | H | H | c-Propyl | CF₂CHF₂ | oil |
| 646 | 3-Cl | 0 | O | H | H | c-Propyl | CF(CH₃)₂ | |
| 647 | H | 0 | O | H | H | c-Butyl | i-Propyl | |
| 648 | 3-Me | 0 | O | H | H | c-Butyl | CHFCH₃ | |
| 649 | 3-F | 1 | 0 | H | H | c-Butyl | CF(CH₃)₂ | |
| 650 | 3-OMe | 0 | S | H | H | c-Pentyl | CHFCH₃ | |
| 651 | 3,5-OMe | 0 | O | H | H | CH₂OMe | CHFCH₃ | |
| 652 | 3-OMe,5-Cl | 0 | S | H | H | s-Butyl | CF(CH₃)₂ | |
| 653 | 3-OEt | 0 | O | H | H | CH₂OEt | CHFCH₃ | |
| 654 | 3-F,5-Me | 0 | S | H | H | t-Butyl | CF₃ | |
| 655 | 3-CF₃ | 0 | O | H | H | Me | CF(CH₃)₂ | |
| 656 | 3-Me,4-F | 1 | O | H | H | Me | CF(CH₃)₂ | |
| 657 | 3-Me | 0 | O | H | H | c-Propyl | CF₂CHF₂ | |
| 658 | 3-0Me | 0 | C(O)O | H | H | i-Propyl | i-Propyl | |
| 659 | 3-Cl,5-Me | 0 | O | H | H | c-Pentyl | CHFCH₃ | |
| 660 | 3-Cl | 0 | O | H | H | Me | CHFCH₃ | |
| 661 | 3-Me | 0 | O | H | H | Et | i-Propyl | |
| 662 | H | 1 | 0 | H | H | c-Propyl | i-Propyl | |
| 664 | 3-Cl | 0 | O | H | Me | c-Butyl | CHFCH₃ | |
| 665 | 3,5-(OMe)₂ | 0 | O | H | H | CH₂-c-Propyl | CF(CH₃)₂ | |
| 666 | 3-OEt | 0 | O | H | H | CH₂-c-Propyl | CF₃ | |
| 667 | 3-F,5-OMe | 0 | SO₂ | H | H | CH₂OMe | CF₂CHF₂ | |
| 668 | 3-Cl | 0 | O | H | H | Me | CF(CH₃)₂ | |
| 669 | 3-F | 0 | O | H | Et | H | CF₂CHF₂ | |
| 670 | 3-Cl | 0 | O | H | H | c-Butyl | CF(CH₃)₂ | |
| 671 | 3-OMe,5-Cl | 0 | O | H | H | CH₂F | i-Propyl | |
| 672 | 3-Cl | 0 | O | H | H | Me | i-Propyl | |
| 673 | 3-F | 0 | O | H | H | Et | CHFCH₃ | |
| 674 | 3-Cl | 0 | O | H | H | c-Butyl | i-Propyl | |
| 675 | 3-OMe,5-Cl | 0 | O | H | Me | CF₃ | CF₂CHF₂ | |
| 676 | H | 1 | 0 | H | H | Et | CHFCH₃ | |
| 677 | 3-F | 0 | O | H | H | c-Propyl | i-Propyl | |
| 678 | 3,5-Me₂ | 0 | O | H | H | CF₃ | CF₂CHF₂ | |
| 679 | 3-F,5-OMe | 0 | O | H | H | CH₂-c-Propyl | CF₃ | |
| 680 | 3-F | 0 | O | H | H | Me | i-Propyl | |
| 681 | H | 0 | O | H | H | c-Propyl | CHFCH₃ | oil |
| 682 | 3-F | 0 | O | H | H | H | i-Propyl | |
| 683 | 3-OEt | 0 | O | H | H | H | CF₂CHF₂ | |
| 684 | 3-Me,4-F | 0 | O | H | H | H | i-Propyl | |
| 685 | 3-Me | 0 | O | H | H | c-Propyl | CHFCH₃ | |
| 686 | 3-OMe | 0 | O | H | Me | Me | CF₃ | |
| 687 | 3-Cl,5-Me | 0 | O | H | H | n-Butyl | CF₂CHF₂ | |
| 688 | 3-F | 0 | O | H | H | Me | CF₂CHF₂ | |
| 688 | 3-F | 0 | O | H | H | Me | CF₂CHF₂ | |
| 689 | 3-Me,4-F | 0 | O | H | H | Me | CHFCH₃ | |
| 690 | 3-Me | 0 | O | H | H | c-Propyl | CF(CH₃)₂ | |
| 691 | 3-Cl | 0 | O | H | H | c-Propyl | i-Propyl | |
| 692 | 3-F | 0 | O | H | H | c-Butyl | CF₂CHF₂ | |
| 693 | 3-OMe | 1 | O | H | H | n-Butyl | CF₂CHF₂ | |
| 694 | 3-Cl,5-Me | 0 | O | H | H | i-Propyl | i-Propyl | |
| 695 | 3-F,5-Me | 0 | O | H | Et | H | CF(CH₃)₂ | |
| 696 | H | 0 | O | H | H | Me | CF₂CHF₂ | oil |
| 697 | 3-Cl | 0 | O | H | H | Et | CF(CH₃)₂ | |
| 698 | H | 0 | O | H | H | c-Butyl | CF₂CHF₂ | oil |
| 699 | 3,5-F | 1 | S | H | H | i-Propyl | i-Propyl | |
| 700 | 3-CF₃ | 0 | SO₂ | H | H | c-Pentyl | CF₃ | |
| 701 | 3-Me | 0 | O | H | H | Me | i-Propyl | |
| 702 | 3-Cl | 0 | O | H | H | Et | CHFCH₃ | |
| 703 | 3-Me | 0 | O | H | H | c-Butyl | i-Propyl | |
| 704 | 3,5-F₂ | 0 | O | H | H | n-Butyl | CF₂CHF₂ | |

TABLE 5
Compounds of the formula (Ie) (=formula I where n = 5, R¹, R²,
R⁵, R⁷, R⁹ and R¹⁰ are each hydrogen and
aryl = phenyl which is substituted as shown in the table)

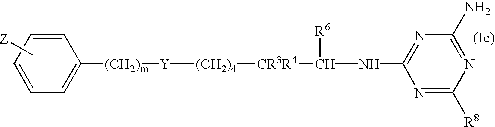

| | | | | | | | | Phys. |
|---|---|---|---|---|---|---|---|---|
| No. | Z | m | Y | R³ | R⁴ | R⁶ | R⁸ | data |
| 705 | 3-F | 0 | O | H | H | Me | CHFCH₃ | |
| 706 | H | 0 | O | H | H | Et | CF(CH₃)₂ | |
| 707 | 3-Me | 0 | O | H | Me | c-Propyl | i-Propyl | |
| 708 | 3-Cl | 0 | O | H | H | c-Propyl | CF₂CHF₂ | |
| 709 | 3-F | 0 | O | H | H | c-Butyl | CHFCH₃ | |
| 710 | 3,5-Me₂ | 0 | O | H | H | t-Butyl | i-Propyl | |
| 711 | 3-Cl,5-Me | 2 | SO₂ | H | H | Me | CHFCF₃ | |
| 712 | 3-F,5-Me | 0 | O | H | H | CH₂F | CF(CH₃)₂ | |
| 713 | H | 0 | O | H | H | Et | CF₂CHF₂ | |
| 714 | 3-F | 0 | O | H | H | c-Propyl | CF(CH₃)₂ | |
| 715 | 3,5-Me₂ | 0 | O | H | H | CH₂F | CHFCH₃ | |
| 716 | 3-F,5-OMe | 0 | O | H | H | CH₂-cButyl | i-Propyl | |
| 717 | H | 0 | O | H | H | Me | CF(CH₃)₂ | |
| 718 | 3-Me | 0 | O | H | H | Me | CF(CH₃)₂ | |
| 719 | 3-Me | 0 | O | H | H | Et | CF₂CHF₂ | |
| 720 | 3-Cl | 0 | O | H | H | Et | CF₂CHF₂ | |
| 721 | H | 0 | O | H | H | c-Butyl | CF(CH₃)₂ | |
| 722 | 3-Me | 0 | O | H | H | c-Butyl | CF(CH₃)₂ | |
| 723 | 3,5-(OMe)₂ | 1 | S | H | Et | H | CF(CH₃)₂ | |
| 724 | 3,5-F₂ | 0 | O | H | H | c-Pentyl | CHFCH₃ | |
| 725 | 3-CF₃ | 2 | S | H | H | i-Propyl | i-Propyl | |
| 726 | H | 0 | O | H | H | Et | i-Propyl | |
| 727 | 3-Cl | 0 | O | H | H | H | CHFCH₃ | |
| 728 | 3,5-Me₂ | 0 | O | H | H | s-Butyl | CHFCF₃ | |
| 729 | 3-F,5-Me | 1 | 0 | H | H | CF₃ | CHFCH₃ | |
| 730 | 3-Me | 0 | O | H | H | Et | CF(CH₃)₂ | |
| 731 | 3-F | 0 | O | H | H | c-Propyl | CF₂CHF₂ | |
| 732 | 3,5-(OMe)₂ | 1 | C(O)O | H | Et | CH₂-c-Butyl | CF₃ | |
| 733 | 3-F,5-OMe | 0 | O | H | H | CH₂OEt | CHFCH₃ | |
| 734 | H | 0 | O | H | H | Me | CHFCH₃ | |
| 735 | 3-Me | 0 | O | H | H | Me | CF₂CHF₂ | |
| 736 | 3-Cl | 0 | O | H | H | Et | i-Propyl | |
| 737 | 3-F | 0 | O | H | H | Et | CF(CH₃)₂ | |
| 738 | H | 0 | O | H | H | c-Butyl | CHFCH₃ | |
| 739 | 3-Me | 0 | O | H | H | H | CF₂CHF₂ | |
| 740 | 3,5-F₂ | 1 | 0 | H | Me | Me | CF₃ | |
| 741 | 3-OMe, | 0 | O | H | H | 1-Butyl | i-Propyl | |
| | 5-Cl | | | | | | | |
| 742 | 3-CF₃ | 0 | O | H | H | n-Butyl | CF₂CHF₂ | |
| 743 | 3-Cl | 0 | O | H | H | Me | CF₂CHF₂ | |
| 744 | H | 0 | O | H | H | c-Propyl | CF(CH₃)₂ | |
| 745 | 3-Cl | 0 | O | H | H | c-Butyl | CF₂CHF₂ | |
| 746 | 3-OEt | 1 | 0 | H | H | CH₂-c-Butyl | i-Propyl | |
| 747 | H | 0 | O | H | H | Me | i-Propyl | |
| 748 | 3-Me | 0 | O | H | H | Me | CHFCH₃ | |
| 749 | 3-F | 0 | O | H | H | H | CF(CH₃)₂ | |
| 750 | 3-Me,4-F | 0 | O | H | H | Me | CF₂CHF₂ | |
| 751 | 3-Me | 1 | 0 | H | H | Et | CHFCH₃ | |
| 752 | 3-F | 0 | O | H | H | Et | i-Propyl | |
| 753 | H | 0 | O | H | H | c-Propyl | CF₂CHF₂ | |
| 754 | 3-Cl | 0 | O | H | H | c-Propyl | CF(CH₃)₂ | |
| 755 | H | 0 | O | H | H | c-Butyl | i-Propyl | |
| 756 | 3-Me | 0 | O | H | H | c-Butyl | CHFCH₃ | |
| 757 | 3-F | 1 | O | H | H | c-Butyl | CF(CH₃)₂ | |
| 758 | 3-OMe | 0 | S | H | H | c-Pentyl | CHFCH₃ | |
| 759 | 3,5-(OMe)₂ | 0 | O | H | H | CH₂OMe | CHFCH₃ | |
| 760 | 3-OMe, 5-Cl | 0 | S | H | H | s-Butyl | CF(CH₃)₂ | |
| 761 | 3-OEt | 0 | O | H | H | CH₂OEt | CHFCH₃ | |
| 762 | 3-F,5-Me | 0 | S | H | H | t-Butyl | CF₃ | |
| 763 | 3-CF₃ | 0 | O | H | H | Me | CF(CH₃)₂ | |
| 764 | 3-Me,4-F | 1 | 0 | H | H | Me | CF(CH₃)₂ | |
| 765 | 3-Me | 0 | O | H | H | c-Propyl | CF₂CHF₂ | |
| 766 | 3-OMe | 0 | C(O)O | H | H | i-Propyl | i-Propyl | |
| 767 | 3-Cl,5-Me | 0 | O | H | H | c-Pentyl | CHFCH₃ | |
| 768 | 3-Cl | 0 | O | H | H | Me | CHFCH₃ | |
| 769 | 3-Me | 0 | O | H | H | Et | i-Propyl | |
| 770 | H | 1 | O | H | H | c-Propyl | i-Propyl | |
| 771 | 3-F | 0 | O | H | H | c-Propyl | CHFCH₃ | |
| 772 | 3-Cl | 0 | O | H | Me | c-Butyl | CHFCH₃ | |
| 773 | 3,5-(OMe)₂ | 0 | O | H | H | CH₂-c-Propyl | CF(CH₃)₂ | |
| 774 | 3-OEt | 0 | O | H | H | CH₂-c-Propyl | CF₃ | |
| 775 | 3-F,5-OMe | 0 | SO₂ | H | H | CH₂OMe | CF₂CHF₂ | |
| 776 | 3-Cl | 0 | O | H | H | Me | CF(CH₃)₂ | |
| 777 | 3-F | 0 | O | H | Et | H | CF₂CHF₂ | |
| 778 | 3-Cl | 0 | O | H | H | c-Butyl | CF(CH₃)₂ | |
| 779 | 3-OMe,5-Cl | O | O | H | H | CH₂F | i-Propyl | |
| 780 | 3-Cl | 0 | O | H | H | Me | i-Propyl | |
| 781 | 3-F | 0 | O | H | H | Et | CHFCH₃ | |
| 782 | 3-Cl | 0 | O | H | H | c-Butyl | i-Propyl | |
| 783 | 3-OMe,5-Cl | 0 | O | H | Me | CF₃ | CF₂CHF₂ | |
| 784 | H | 1 | O | H | H | Et | CHFCH₃ | |
| 785 | 3-F | 0 | O | H | H | c-Propyl | i-Propyl | |
| 786 | 3,5-Me2 | 0 | O | H | H | CF₃ | CF₂CHF₂ | |
| 787 | 3-F,5-OMe | 0 | O | H | H | CH₂-c-Propyl | CF₃ | |
| 788 | 3-F | 0 | O | H | H | Me | i-Propyl | |
| 789 | H | 0 | O | H | H | c-Propyl | CHFCH₃ | |
| 790 | 3-F | 0 | O | H | H | H | i-Propyl | |
| 791 | 3-OEt | 0 | O | H | H | H | CF₂CHF₂ | |
| 792 | 3-Me,4-F | 0 | O | H | H | H | i-Propyl | |
| 793 | 3-Me | 0 | O | H | H | c-Propyl | CHFCH₃ | |
| 794 | 3-OMe | 0 | O | H | Me | Me | CF₃ | |
| 795 | 3-Cl,5-Me | 0 | O | H | H | n-Butyl | CF₂CHF₂ | |
| 796 | 3-F | 0 | O | H | H | Me | CF₂CHF₂ | |
| 797 | 3-Me,4-F | 0 | O | H | H | Me | CHFCH₃ | |
| 798 | 3-Me | 0 | O | H | H | c-Propyl | CF(CH₃)₂ | |
| 799 | 3-Cl | 0 | O | H | H | c-Propyl | i-Propyl | |
| 800 | 3-F | 0 | O | H | H | c-Butyl | CF₂CHF₂ | |
| 801 | 3-OMe | 1 | 0 | H | H | n-Butyl | CF₂CHF₂ | |
| 802 | 3-Cl,5-Me | 0 | O | H | H | i-Propyl | i-Propyl | |
| 803 | 3-F,5-Me | 0 | O | H | Et | H | CF(CH₃)₂ | |
| 804 | H | 0 | O | H | H | Me | CF₂CHF₂ | |
| 805 | 3-Cl | 0 | O | H | H | Et | CF(CH₃)₂ | |
| 806 | H | 0 | O | H | H | c-Butyl | CF₂CHF₂ | |
| 807 | 3,5-F₂ | 1 | S | H | H | i-Propyl | i-Propyl | |
| 808 | 3-CF₃ | 0 | SO₂ | H | H | c-Pentyl | CF₃ | |
| 809 | 3-Me | 0 | O | H | H | Me | i-Propyl | |
| 810 | 3-Cl | 0 | O | H | H | Et | CHFCH, | |
| 811 | 3-Me | 0 | O | H | H | c-Butyl | i-Propyl | |
| 812 | 3,5-F₂ | 0 | O | H | H | n-Butyl | CF₂CHF₂ | |

TABLE 6
Compounds of the formula (If) (=compounds of the formula (I)
in which m = 0, Y = O, (CR³R⁴)ₙ-CR⁵R⁶ =
cyclohexane-1,3-diyl, R⁷ = H, R⁹ = H and aryl =
phenyl which is substituted as shown in the table)

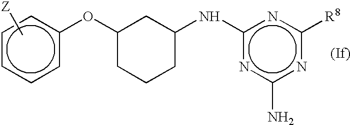

| No. | Z | R⁸ | Phys. data |
|---|---|---|---|
| 813 | 3,4-F₂ | i-Propyl | |
| 814 | 3,4-F₂ | CF(CH₃)₂ | |
| 815 | 3,4-F₂ | CHFCH₃ | |
| 816 | 3,4-F₂ | CF₂CHF₂ | |
| 817 | 3,5-Me₂ | CF(CH₃)₂ | |
| 818 | 3,5-Me₂ | CHFCH₃ | |
| 819 | 3,5-Me₂ | CF(CH₃)₂ | |
| 820 | 3,5-Me₂ | CHFCH₃ | |
| 821 | 3-Cl | CF(CH₃)₂ | |
| 822 | 3-Cl | CHFCH₃ | |
| 823 | 3-Cl,5-F | CF(CH₃)₂ | |
| 824 | 3-Cl,5-F | CHFCH₃ | |
| 825 | 3-F | CF(CH₃)₂ | |
| 826 | 3-F | CHFCH₃ | |
| 827 | 3-Me | CCl(CH₃)3 | |
| 828 | 3-Me | Et | |
| 829 | 3-Me | CF₂CF₃ | |
| 830 | 3-Me | CF(CH₃)₂ | |
| 831 | 3-Me | CHFCH₃ | |
| 832 | 3-Me,4-F | CF(CH₃)₂ | |
| 833 | 3-Me,4-F | CHFCH₃ | |
| 834 | 4-F | 1-Propyl | |
| 835 | 4-F | CF(CH₃)₂ | |
| 836 | 4-F | CHFCH₃ | |
| 837 | 4-F | CF₂CHF₂ | |
| 838 | H | CCl(CH₃)₂ | |
| 839 | H | Me | |
| 840 | H | CF(CH₃)₂ | |
| 841 | H | CHFCH₃ | |

TABLE 7
Compounds of the formula (Ig) (=compounds of the formula (I),
in which m = 0, Y = O, (CR³R⁴)ₙ-CR⁵R⁶ =
—CH₂-1,2-cycloC₆H₁₀—, R⁷ = R⁹ =R¹⁰ =
H and aryl = phenyl which is substituted as shown in the table)

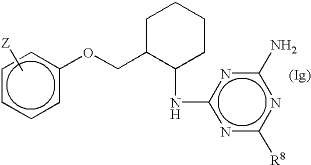

| No. | Z | R8 | Phys. data |
|---|---|---|---|
| 842 | 3,4-F₂ | i-Propyl | |
| 843 | 3,4-F₂ | CF(CH₃)₂ | |
| 844 | 3,4-F₂ | CHFCH₃ | |
| 845 | 3,4-F₂ | CF₂CHF₂ | |
| 846 | 3,5-Me₂ | CF(CH₃)₂ | |
| 847 | 3,5-Me₂ | CHFCH₃ | |
| 848 | 3,5-Me₂ | CF(CH₃)₂ | |
| 849 | 3,5-Me₂ | CHFCH₃ | |
| 850 | 3-Cl | CF(CH₃)₂ | |
| 851 | 3-Cl | CHFCH₃ | |
| 852 | 3-Cl,5-F | CF(CH₃)₂ | |
| 853 | 3-Cl,5-F | CHFCH₃ | |
| 854 | 3-F | CF(CH₃)₂ | |
| 855 | 3-F | CHFCH₃ | |
| 856 | 3-Me | CCl(CH₃)₃ | |
| 857 | 3-Me | Et | |
| 858 | 3-Me | CF₂CF₃ | |
| 859 | 3-Me | CF(CH₃)₂ | |
| 860 | 3-Me | CHFCH₃ | |
| 861 | 3-Me,4-F | CF(CH₃)₂ | |
| 862 | 3-Me,4-F | CHFCH₃ | |
| 863 | 4-F | 1-Propyl | |
| 864 | 4-F | CF(CH₃)₂ | |
| 865 | 4-F | CHFCH₃ | |
| 868 | 4-F | CF₂CHF₂ | |
| 867 | H | CCl(CH₃)₂ | |
| 868 | H | Me | |
| 869 | H | CF(CH₃)₂ | resin |
| 870 | H | CHFCH₃ | resin |

TABLE 8
Compounds of the formula (Ih) (=formula (I), in which m = 0, Y = O,
(CR³R⁴)ₙ—CR⁵R⁶ = —CH₂1,2-cyclopentyl-,
R⁷, R⁹, R¹⁰ are each H and aryl = phenyl
which is substituted as shown in the table)

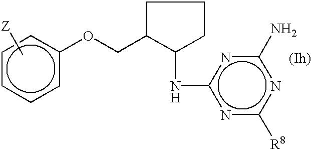

| No. | Z | R8 | Phys. data |
|---|---|---|---|
| 871 | 3,4-F₂ | i-Propyl | |
| 872 | 3,4-F₂ | CF(CH₃)₂ | |
| 873 | 3,4-F₂ | CHFCH₃ | |
| 874 | 3,4-F₂ | CF₂CHF₂ | |
| 875 | 3,5-Me₂ | CF(CH₃)₂ | |
| 876 | 3,5-Me₂ | CHFCH₃ | |
| 877 | 3,5-Me₂ | CF(CH₃)₂ | |
| 878 | 3,5-Me₂ | CHFCH₃ | |
| 879 | 3-Cl | CF(CH₃)₂ | |
| 880 | 3-Cl | CHFCH₃ | |
| 881 | 3-Cl,5-F | CF(CH₃)₂ | |
| 882 | 3-Cl,5-F | CHFCH₃ | |
| 883 | 3-F | CF(CH₃)₂ | |
| 884 | 3-F | CHFCH₃ | |
| 885 | 3-Me | CCl(CH₃)₃ | |
| 886 | 3-Me | Et | |
| 887 | 3-Me | CF₂CF₃ | |
| 888 | 3-Me | CF(CH₃)₂ | |
| 889 | 3-Me | CHFCH₃ | |
| 890 | 3-Me,4-F | CF(CH₃)₂ | |
| 891 | 3-Me,4-F | CHFCH₃ | |
| 892 | 4-F | i-Propyl | |
| 893 | 4-F | CF(CH₃)₂ | |
| 894 | 4-F | CHFCH₃ | |
| 895 | 4-F | CF₂CHF₂ | |
| 896 | H | CCl(CH₃)₂ | |
| 897 | H | Me | |
| 898 | H | CF(CH₃)₂ | |
| 899 | H | CHFCH₃ | |

TABLE 9
Compounds of the formula (Ik) (=formula (I), in which m = 1,
n = 1, R¹ and R² are each H, (CR³R⁴) =
carbonyl; R⁵, R⁷, R⁹, R¹⁰ are each H and
aryl = phenyl which is substituted as shown in the table)

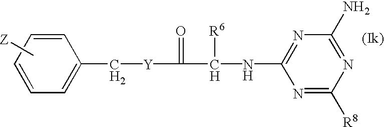

| No. | Z | Y | R⁶ | R⁸ | Phys. data |
|---|---|---|---|---|---|
| 900 | 3-F | O | Me | CHFCH₃ | |
| 901 | H | O | Et | CF(CH₃)₂ | |
| 902 | 3-Me | O | c-Propyl | i-Propyl | |
| 903 | 3-Cl | O | CH₂CH₂SCH₃ | CF₂CHF₂ | |
| 904 | 3-F | N | c-Butyl | CHFCH₃ | |
| 905 | 3,5-Me₂ | N | t-Butyl | i-Propyl | |
| 906 | 3-Cl,5-Me | N | Me | CHFCF₃ | |
| 907 | 3-F,5-Me | O | Benzyl | CF(CH₃)₂ | |
| 908 | H | N | Et | CF₂CHF₂ | |
| 909 | 3-F | O | c-Propyl | CF(CH₃)₂ | |
| 910 | 3,5-Me₂ | N | CH₂F | CHFCH₃ | |
| 911 | 3,5-Me₂ | N | s-Butyl | CHFCH₃ | |
| 912 | 3-F,5-OMe | O | CH₂-c-Butyl | 1-Propyl | |
| 913 | H | N | Me | CF(CH₃)₂ | |
| 914 | 3-Me | O | CH₂CH₂SCH₃ | CF(CH₃)₂ | |
| 915 | 3-Me | N | Et | CF₂CHF₂ | |
| 916 | 3-Cl | O | Et | CF₂CHF₂ | |
| 917 | H | O | c-Butyl | CF(CH₃)₂ | |
| 918 | 3-Me | O | c-Butyl | CF(CH₃)₂ | |
| 919 | H | N | Et | Me | |
| 920 | 3,5-F₂ | O | c-Pentyl | CHFCH₃ | |
| 921 | 3-CF₃ | O | i-Propyl | i-Propyl | |
| 922 | H | N | Et | i-Propyl | |
| 923 | 3-Cl | O | Benzyl | CHFCH₃ | |
| 924 | H | O | CH₂CH₂SCH₃ | Me | |
| 925 | 3-F,5-Me | O | CF₃ | CHFCH₃ | |
| 926 | 3-Me | N | Me | CF(CH₃)₂ | m.p. |
| | | | | | 98° C. |
| 927 | 3-F | N | c-Propyl | CF₂CHF₂ | |
| 928 | 3,5-(OMe)₂ | N | CH₂-c-Butyl | CF₃ | |
| 929 | 3-F,5-OMe | N | CH₂OEt | CHFCH₃ | |
| 930 | H | N | H | CHFCH₃ | m.p. |
| | | | | | 123° C. |
| 931 | 3-Me | N | Me | CF₂CHF₂ | |
| 932 | 3-Cl | N | Et | i-Propyl | |
| 933 | 3-F | N | Et | CF(CH₃)₂ | |
| 934 | H | N | Et | Et | |
| 935 | 3-Me | N | Benzyl | CF₂CHF₂ | |
| 938 | 3,5-F₂ | N | Me | CF₃ | |
| 937 | 3-OMe,5-Cl | O | t-Butyl | i-Propyl | |
| 938 | 3-CF₃ | O | i-Butyl | CF₂CHF₂ | |
| 939 | 3-Cl | O | Me | CF₂CHF₂ | |
| 940 | H | O | c-Propyl | CF(CH₃)₂ | |
| 941 | 3-Cl | O | c-Butyl | CF₂CHF₂ | |
| 942 | 3-OEt | N | CH₂-c-Butyl | i-Propyl | |
| 943 | H | N | Me | i-Propyl | |
| 944 | 3-Me | N | Me | CHFCH₃ | |
| 945 | 3-F | N | H | CF(CH₃)₂ | |
| 946 | 3-Me,4-F | N | Me | CF₂CHF₂ | |
| 947 | 3-Me | N | Et | CHFCH₃ | |
| 948 | 3-F | N | Et | i-Propyl | |
| 949 | H | N | c-Propyl | CF₂CHF₂ | |
| 950 | 3-Cl | N | c-Propyl | CF(CH₃)₂ | |
| 951 | H | N | s-Butyl | Me | |
| 952 | 3-Me | N | c-Butyl | CHFCH₃ | |
| 953 | 3-F | N | c-Butyl | CF(CH₃)₂ | |
| 954 | 3-OMe | N | c-Pentyl | CHFCH₃ | |
| 955 | 3,5-(OMe)₂ | N | CH2OMe | CHFCH₃ | |
| 956 | 3-OMe,5-Cl | N | CH₂CH₂SCH₃ | CF(CH₃)₂ | |
| 957 | 3-OEt | N | CH₂OEt | CHFCH₃ | |
| 958 | 3-F,5-Me | N | 1-Butyl | CF₃ | |
| 959 | 3-CF₃ | N | Me | CF(CH₃)₂ | |
| 960 | 3-Me,4-F | N | Me | CF(CH₃)₂ | |
| 961 | 3-Me | N | c-Propyl | CF₂CHF₂ | |
| 962 | 3-OMe | N | Benzyl | i-Propyl | |
| 963 | 3-Cl,5-Me | O | c-Pentyl | CHFCH₃ | |
| 964 | 3-Cl | O | Me | CHFCH₃ | |
| 965 | 3-Me | O | Et | i-Propyl | |
| 966 | H | O | c-Propyl | i-Propyl | |
| 967 | 3-F | N | c-Propyl | CHFCH₃ | |
| 968 | 3-Cl | N | c-Butyl | CHFCH₃ | |
| 969 | 3,5-(OMe)₂ | N | CH₂-cPropyl | CF(CH₃)₂ | |
| 970 | 3-OEt | N | i-ButyI | CF₃ | |
| 971 | 3-F,5-OMe | N | CH₂OMe | CF₂CHF₂ | |
| 972 | 3-Cl | N | Me | CF(CH₃)₂ | |
| 973 | 3-F | N | H | CF₂CHF₂ | |
| 974 | 3-Cl | N | c-Butyl | CF(CH₃)₂ | |
| 975 | 3-OMe,5-Cl | N | CH₂F | i-Propyl | |
| 976 | 3-Cl | N | Me | i-Propyl | |
| 977 | 3-F | N | CH₂CH₂SCH₃ | CHFCH₃ | |
| 978 | 3-Ct | N | c-Butyl | i-Propyl | |
| 979 | 3-OMe,5-Cl | N | CF₃ | CF₂CHF₂ | |
| 980 | H | N | Et | CHFCH₃ | |
| 981 | 3-F | N | c-Propyl | i-Propyl | |
| 982 | 3,5-Me₂ | N | CF₃ | CF₂CHF₂ | |
| 983 | 3-F,5-OMe | O | CH₂-c-Propyl | CF₃ | |
| 984 | 3-F | O | Me | i-Propyl | |
| 985 | H | O | c-Propyl | CHFCH₃ | |
| 986 | 3-F | O | H | i-Propyl | |
| 987 | H | N | H | i-Propyl | m.p. |
| | | | | | 143° C. |
| 988 | 3-Me,4-F | O | H | i-Propyl | |
| 989 | 3-Me | N | c-Propyl | CHFCH₃ | |
| 990 | 3-OMe | N | Me | CF₃ | |
| 991 | 3-Cl,5-Me | N | n-Butyl | CF₂CHF₂ | |
| 992 | 3-F | N | Me | CF₂CHF₂ | |
| 993 | 3-Me,4-F | N | Me | CHFCH₃ | |
| 994 | 3-Me | N | c-Propyl | CF(CH₃)₂ | |
| 995 | 3-Cl | N | c-Propyl | i-Propyl | |
| 996 | 3-F | N | c-Butyl | CF₂CHF₂ | |
| 997 | 3-OMe | N | n-Butyl | CF₂CHF₂ | |
| 998 | 3-Cl,5-Me | N | Benzyl | i-Propyl | |
| 999 | 3-F,5-Me | N | H | CF(CH₃)₂ | |
| 1000 | H | N | Me | CF₂CHF₂ | |
| 1001 | 3-Cl | N | Et | CF(CH₃)₂ | |
| 1002 | H | N | c-Butyl | CF₂CHF₂ | |
| 1003 | 3,5-F | N | i-Propyl | i-Propyl | |
| 1004 | 3-CF₃ | N | c-Pentyl | CF₃ | |
| 1005 | 3-Me | N | Me | i-Propyl | m.p. |
| | | | | | 112° C. |
| 1006 | 3-Cl | N | Et | CHFCH₃ | |
| 1007 | 3-Me | N | c-Butyl | i-Propyl | |
| 1008 | 3,5-F₂ | N | n-Butyl | CF₂CHF₂ | |

Table 10
Compounds of the formula (Im) (=formula (I), in which m = 0,
n = 1, Y = —NH—CO—NH—, R³ and R⁴ are each
H; R⁵, R⁷, R⁹, R¹⁰ are each H and aryl =
phenyl which is substituted as shown in the table)

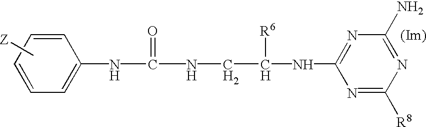

| No. | Z | R⁶ | R⁸ | Phys. data |
|---|---|---|---|---|
| 1009 | 3-F | Me | CHFCH₃ | |
| 1010 | H | Et | CF(CH₃)₂ | |
| 1011 | 3-Me | c-Propyl | i-Propyl | |
| 1012 | 3-Cl | Pentyl | CF₂CHF₂ | |
| 1013 | 3-F | c-Butyl | CHFCH₃ | |
| 1014 | 3,5-Me₂ | t-Butyl | 1-Propyl | |
| 1015 | 3-Cl,5-Me | Me | CHFCF₃ | |
| 1016 | 3-F,5-Me | CH₂OEt | CF(CH₃)₂ | |
| 1017 | H | Et | CF₂CHF₂ | |
| 1018 | 3-F | c-Propyl | CF(CH₃)₂ | |
| 1019 | 3,5-Me₂ | CH₂F | CHFCH₃ | |
| 1020 | 3,5-Me₂ | s-Butyl | CHFCH₃ | |
| 1021 | 3-F,5-OMe | CH₂-c-Butyl | i-Propyl | |
| 1022 | H | Me | CF(CH₃)₂ | |
| 1023 | 3-Me | CH₂OMe | CF(CH₃)₂ | |
| 1024 | 3-Me | Et | CF₂CHF₂ | |
| 1025 | 3-Cl | Et | CF₂CHF₂ | |
| 1026 | H | c-Butyl | CF(CH₃)₂ | |
| 1027 | 3-Me | c-Butyl | CF(CH₃)₂ | |
| 1028 | H | Et | Me | |
| 1029 | 3,5-F₂ | c-Pentyl | CHFCH₃ | |
| 1030 | 3-CF₃ | i-Propyl | i-Propyl | |
| 1031 | H | Et | i-Propyl | |
| 1032 | 3-Cl | CH₂OEt | CHFCH₃ | |
| 1033 | H | CH₂OMe | Me | |
| 1034 | 3-F,5-Me | CF₃ | CHFCH₃ | |
| 1035 | 3-Me | Me | CF(CH₃)₂ | |
| 1036 | 3-F | c-Propyl | CF₂CHF₂ | |
| 1037 | 3,5-(OMe)₂ | CH₂-c-Butyl | CF₃ | |
| 1038 | 3-F,5-OMe | CH₂OEt | CHFCH₃ | |
| 1039 | H | H | CHFCH₃ | |
| 1040 | 3-Me | Me | CF₂CHF₂ | |
| 1041 | 3-Cl | Et | i-Propyl | |
| 1042 | 3-F | Et | CF(CH₃)₂ | |
| 1043 | H | Et | Et | |
| 1044 | 3-Me | Pentyl | CF₂CHF₂ | |
| 1045 | 3,5-F₂ | Me | CF₃ | |
| 1046 | 3-OMe,5-Cl | t-Butyl | i-Propyl | |
| 1047 | 3-CF₃ | i-Butyl | CF₂CHF₂ | |
| 1048 | 3-Cl | Me | CF₂CHF₂ | |
| 1049 | H | c-Propyl | CF(CH₃)₂ | |
| 1050 | 3-Cl | c-Butyl | CF₂CHF₂ | |
| 1051 | 3-OEt | CH₂-c-Butyl | i-Propyl | |
| 1052 | H | Me | i-Propyl | |
| 1053 | 3-Me | Me | CHFCH₃ | |
| 1054 | 3-F | H | CF(CH₃)₂ | |
| 1055 | 3-Me,4-F | Me | CF₂CHF₂ | |
| 1056 | 3-Me | Et | CHFCH₃ | |
| 1057 | 3-F | Et | i-Propyl | |
| 1058 | H | c-Propyl | CF₂CHF₂ | |
| 1059 | 3-Cl | c-Propyl | CF(CH₃)₂ | |
| 1060 | H | s-Butyl | Me | |
| 1061 | 3-Me | c-Butyl | CHFCH₃ | |
| 1062 | 3-F | c-Butyl | CF(CH₃)₂ | |
| 1063 | 3-OMe | c-Pentyl | CHFCH₃ | |
| 1064 | 3,5-(OMe)₂ | CH₂OMe | CHFCH₃ | |
| 1065 | 3-OMe,5-Cl | H | CF(CH₃)₂ | |
| 1066 | 3-QEt | CH₂OEt | CHFCH₃ | |
| 1067 | 3-F,5-Me | 1-Butyl | CF₃ | |
| 1068 | 3-CF₃ | Me | CF(CH₃)₂ | |
| 1069 | 3-Me,4-F | Me | CF(CH₃)₂ | |
| 1070 | 3-Me | c-Propyl | CF₂CHF₂ | |
| 1071 | 3-OMe | Pentyl | i-Propyl | |
| 1072 | 3-Cl,5-Me | c-Pentyl | CHFCH₃ | |
| 1073 | 3-Cl | Me | CHFCH₃ | |
| 1074 | 3-Me | Et | i-Propyl | |
| 1075 | H | c-Propyl | i-P ropyl | |
| 1076 | 3-F | c-Propyl | CHFCH₃ | |
| 1078 | 3,5-(OMe)₂ | CH₂-cPropyl | CF(CH₃)₂ | |
| 1079 | 3-CEt | 1-Butyl | CF₃ | |
| 1080 | 3-F,5-OMe | CH₂OMe | CF₂CHF₂ | |
| 1081 | 3-Cl | Me | CF(CH₃)₂ | |
| 1082 | 3-F | H | CF₂CHF₂ | |
| 1083 | 3-Cl | c-Butyl | CF(CH₃)₂ | |
| 1084 | 3-OMe,5-Cl | CH₂F | i-Propyl | |
| 1085 | 3-Cl | Me | i-Propyl | |
| 1086 | 3-F | CH₂OEt | CHFCH₃ | |
| 1087 | 3-Cl | c-Butyl | i-Propyl | |
| 1088 | 3-OMe,5-Cl | CF₃ | CF₂C HF₂ | |
| 1089 | H | Et | CHFCH₃ | |
| 1090 | 3-F | c-Propyl | i-Propyl | |
| 1091 | 3,5-Me₂ | CF₃ | CF₂CHF₂ | |
| 1092 | 3-F,5-OMe | CH₂-c-Propyl | CF₃ | |
| 1093 | 3-F | Me | i-Propyl | |
| 1094 | H | c-Propyl | CHFCH₃ | |
| 1095 | 3-F | H | i-Propyl | |
| 1096 | H | H | i-Propyl | |
| 1097 | 3-Me,4-F | H | i-Propyl | |
| 1098 | 3-Me | c-Propyl | CHFCH₃ | |
| 1099 | 3-OMe | Me | CF₃ | |
| 1100 | 3-Cl,5-Me | n-Butyl | CF₂CHF₂ | |
| 1101 | 3-F | Me | CF₂CHF₂ | |
| 1102 | 3-Me,4-F | Me | CHFCH₃ | |
| 1103 | 3-Me | c-Propyl | CF(CH₃)₂ | |
| 1104 | 3-Cl | c-Propyl | i-Propyl | |
| 1105 | 3-F | c-Butyl | CF₂CHF₂ | |
| 1106 | 3-OMe | n-Butyl | CF₂CHF₂ | |
| 1107 | 3-Cl,5-Me | Hexyl | i-Propyl | |
| 1108 | 3-F,5-Me | H | CF(CH₃)₂ | |
| 1109 | H | Me | CF₂CHF₂ | |
| 1110 | 3-Cl | Et | CF(CH₃)₂ | |
| 1111 | H | c-Butyl | CF₂CHF₂ | |
| 1112 | 3,5-F₂ | i-Propyl | i-Propyl | |
| 1113 | 3-CF₃ | c-Pentyl | CF₃ | |
| 1114 | 3-Me | Me | i-Propyl | |
| 1115 | 3-Cl | Et | CHFCH₃ | |
| 1116 | 3-Me | c-Butyl | i-Propyl | |
| 1117 | 3,5-F₂ | n-Butyl | CF₂CHF₂ | |

TABLE 11
Compounds of the formula (In) (=formula (I), in which m = 0, n = 1,
Y = —NH—CO—O—, R³ and R⁴ are each H; R⁵, R⁷,
R⁹, R¹⁰ are each H and aryl = phenyl which
is substituted as shown in the table)

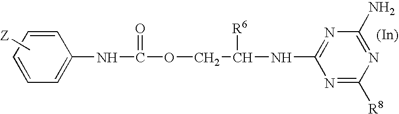

| No. | Z | R⁶ | R⁸ | Phys. data |
|---|---|---|---|---|
| 1118 | 3-Me,4-F | H | i-Propyl | |
| 1119 | 3-Me | c-Propyl | CHFCH₃ | |
| 1120 | 3-OMe | Me | CF₃ | |
| 1121 | 3-Cl | Pentyl | CF₂CHF₂ | |
| 1122 | 3-F | c-Butyl | CHFCH₃ | |
| 1123 | 3,5-Me₂ | 1-Butyl | 1-Propyl | |
| 1124 | 3-Cl,5-Me | Me | CHFCF₃ | |
| 1125 | 3-F,5-Me | CH₂OEt | CF(CH₃)₂ | |
| 1126 | H | Et | CF₂CHF₂ | |
| 1127 | 3-F | c-Propyl | CF(CH₃)₂ | |
| 1128 | 3-Cl | Et | CF(CH₃)₂ | |
| 1129 | H | c-Butyl | CF₂CHF₂ | |
| 1130 | 3,5-F₂ | 1-Propyl | 1-Propyl | |
| 1131 | 3-CF₃ | c-Pentyl | CF₃ | |
| 1132 | 3-Me | Me | i-Propyl | |
| 1133 | 3-F | Me | CHFCH₃ | |
| 1134 | H | Et | CF(CH₃)₂ | |
| 1135 | 3-Me | c-Propyl | i-Propyl | |
| 1136 | 3-Me | Et | CF₂CHF₂ | |
| 1137 | 3-Cl | Et | CF₂CHF₂ | |
| 1138 | H | c-Butyl | CF(CH₃)₂ | |
| 1139 | 3-Me | c-Butyl | CF(CH₃)₂ | |
| 1140 | H | Et | Me | |
| 1141 | 3,5-F₂ | c-Pentyl | CHFCH₃ | |
| 1142 | 3-CF₃ | i-Propyl | 1-Propyl | |
| 1143 | H | Et | 1-Propyl | |
| 1144 | 3-Cl | CH₂OEt | CHFCH₃ | |
| 1145 | H | CH₂OMe | Me | |
| 1146 | 3-F,5-Me | CF₃ | CHFCH₃ | |
| 1147 | 3,5-Me₂ | CH₂F | CHFCH₃ | |
| 1148 | 3,5-Me₂ | s-Butyl | CHFCH₃ | |
| 1149 | 3-F,5-OMe | CH₂-c-Butyl | i-Propyl | |
| 1150 | H | Me | CF(CH₃)₂ | |
| 1151 | 3-Me | CH₂OMe | CF(CH₃)₂ | |
| 1152 | 3-Me | Me | CF(CH₃)₂ | |
| 1153 | 3-F | c-Propyl | CF₂CHF₂ | |
| 1154 | 3,5-(OMe)₂ | CH₂-c-Butyl | CF₃ | |
| 1155 | 3-F,5-OMe | CH₂OEt | CHFCH₃ | |
| 1156 | H | H | CHFCH₃ | |
| 1157 | 3-Me | Me | CF₂CHF₂ | |
| 1158 | 3-Cl | Et | 1-Propyl | |
| 1159 | 3-F | Et | CF(CH₃)₂ | |
| 1160 | H | Et | Et | |
| 1161 | 3-Me | Pentyl | CF₂CHF₂ | |
| 1162 | 3,5-F₂ | Me | CF₃ | |
| 1163 | 3-OMe,5-Cl | t-Butyl | 1-Propyl | |
| 1164 | 3-CF₃ | i-Butyl | CF₂CHF₂ | |
| 1165 | 3-Cl | Me | CF₂CHF₂ | |
| 1166 | H | c-Propyl | CF(CH₃)₂ | |
| 1167 | 3-Cl | c-Butyl | CF₂CHF₂ | |
| 1168 | 3-OEt | CH₂-c-Butyl | i-Propyl | |
| 1169 | H | Me | i-Propyl | |
| 1170 | 3-Me | Me | CHFCH₃ | |
| 1171 | 3-F | H | CF(CH₃)₂ | |
| 1172 | 3-Me,4-F | Me | CF₂CHF₂ | |
| 1173 | 3-Me | Et | CHFCH₃ | |
| 1174 | 3-F | Et | i-Propyl | |
| 1175 | H | c-Propyl | CF₂CHF₂ | |
| 1176 | 3-Cl | c-Propyl | CF(CH₃)₂ | |
| 1177 | H | s-Butyl | Me | |
| 1178 | 3-Me | c-Butyl | CHFCH₃ | |
| 1179 | 3-F | c-Butyl | CF(CH₃)₂ | |
| 1180 | 3-OMe | c-Pentyl | CHFCH₃ | |
| 1181 | 3,5-(OMe)₂ | CH₂OMe | CHFCH₃ | |
| 1182 | 3-OMe,5-Cl | H | CF(CH₃)₂ | |
| 1183 | 3-OEt | CH₂OEt | CHFCH₃ | |
| 1184 | 3-F,5-Me | t-Butyl | CF₃ | |
| 1185 | 3-CF₃ | Me | CF(CH₃)₂ | |
| 1186 | 3-Me,4-F | Me | CF(CH₃)₂ | |
| 1187 | 3-Me | c-Propyl | CF₂CHF₂ | |
| 1188 | 3-OMe | Pentyl | i-Propyl | |
| 1189 | 3-Cl,5-Me | c-Pentyl | CHFCH₃ | |
| 1190 | 3-Cl | Me | CHFCH₃ | |
| 1191 | 3-Me | Et | i-Propyl | |
| 1192 | H | c-Propyl | i-Propyl | |
| 1193 | 3-F | c-Propyl | CHFCH₃ | |
| 1194 | 3-Cl | c-Butyl | CHFCH₃ | |
| 1195 | 3,5-(OMe)₂ | CH₂-cPropyl | CF(CH₃)₂ | |
| 1196 | 3-OEt | 1-Butyl | CF₃ | |
| 1197 | 3-F,5-OMe | CH₂OMe | CF₂CHF₂ | |
| 1198 | 3-Cl | Me | CF(CH₃)₂ | |
| 1199 | 3-F | H | CF₂CHF₂ | |
| 1200 | 3-Cl | c-Butyl | CF(CH₃)₂ | |
| 1201 | 3-OMe,5-Cl | CH₂F | i-Propyl | |
| 1202 | 3-Cl | Me | i-Propyl | |
| 1203 | 3-F | CH₂OEt | CHFCH₃ | |
| 1204 | 3-Cl | c-Butyl | i-Propyl | |
| 1205 | 3-OMe,5-Cl | CF₃ | CF₂CHF₂ | |
| 1206 | H | Et | CHFCH₃ | |
| 1207 | 3-F | c-Propyl | i-Propyl | |
| 1208 | 3,5-Me₂ | CF₃ | CF₂CHF₂ | |
| 1209 | 3-F,5-OMe | CH₂-c-Propyl | CF₃ | |
| 1210 | 3-F | Me | i-Propyl | |
| 1211 | H | c-Propyl | CHFCH₃ | |
| 1212 | 3-F | H | i-Propyl | |
| 1213 | H | H | i-Propyl | |
| 1214 | 3-Cl | Et | CHFCH₃ | |
| 1215 | 3-Me | c-Butyl | i-Propyl | |
| 1216 | 3,5-F₂ | n-Butyl | CF₂CHF₂ | |
| 1217 | 3-Cl,5-Me | n-Butyl | CF₂CHF₂ | |
| 1218 | 3-F | Me | CF₂CHF₂ | |
| 1219 | 3-Me,4-F | Me | CHFCH₃ | |
| 1220 | 3-Me | c-Propyl | CF(CH₃)₂ | |
| 1221 | 3-Cl | c-Propyl | i-Propyl | |
| 1222 | 3-F | c-Butyl | CF₂CHF₂ | |
| 1223 | 3-OMe | n-Butyl | CF₂CHF₂ | |
| 1224 | 3-Cl,5-Me | Hexyl | i-Propyl | |
| 1225 | 3-F,5-Me | H | CF(CH₃)₂ | |
| 1226 | H | Me | CF₂CHF₂ | |

TABLE 12
Compounds of the formula (Io) (=formula (I), in which m = 1, n = 1,
Y = —O—N═, R³ = linkage point for double bond,
R⁴ = H; R⁵; R⁷, R⁹, R¹⁰ are each H and
aryl = phenyl which is substituted as shown in the table)

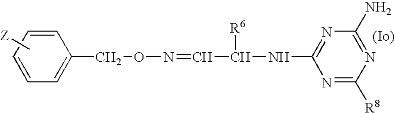

| No. | Z | R⁹ | R⁸ | Phys. data |
|---|---|---|---|---|
| 1227 | 3-F,5-OMe | CH₂OMe | CF₂CHF₂ | |
| 1228 | 3-Cl | Me | CF(CH₃)₂ | |
| 1229 | 3-F | H | CF₂CHF₂ | |
| 1230 | 3-Cl | Pentyl | CF₂CHF₂ | |
| 1231 | 3-F | c-Butyl | CHFCH₃ | |
| 1232 | 3,5-Me₂ | 1-Butyl | i-propyl | |
| 1233 | 3-Cl,5-Me | Me | CHFCF₃ | |
| 1234 | 3,5-Me₂ | H | CHFCH₃ | resin |
| 1235 | 3,5-Me₂ | H | Et | resin |
| 1236 | 3,5-Me₂ | H | CF(CH₃)₂ | resin |
| 1237 | H | Et | CF₂CHF₂ | |
| 1238 | 3-F | c-Propyl | CF(CH₃)₂ | |
| 1239 | 3-Cl | Et | CF(CH₃)₂ | |
| 1240 | H | c-Butyl | CF₂CHF₂ | |
| 1241 | 3,5-F₂ | i-Propyl | i-propyl | |
| 1242 | 3-CF₃ | c-Pentyl | CF₃ | |
| 1243 | 3-Me | Me | i-propyl | |
| 1244 | 3-F | Me | CHFCH₃ | |
| 1245 | H | Et | CF(CH₃)₂ | |
| 1246 | 3-Me | c-Propyl | i-propyl | |
| 1247 | 3-Me | Et | CF₂CHF₂ | |
| 1248 | 3-Cl | Et | CF₂CHF₂ | |
| 1249 | H | c-Butyl | CF(CH₃)₂ | |
| 1250 | 3-Me | c-Butyl | CF(CH₃)₂ | |
| 1251 | 3,5-Me₂ | H | CH(CH₃)₂ | resin |
| 1252 | H | H | CF(CH₃)₂ | resin |
| 1253 | H | H | CH(CH₃)₂ | resin |
| 1254 | H | Et | Me | |
| 1255 | 3-Me,4-F | H | i-Propyl | |
| 1256 | 3-Me | c-Propyl | CHFCH₃ | |
| 1257 | 3-OMe | Me | CF₃ | |
| 1258 | 3,5-F₂ | c-Pentyl | CHFCH₃ | |
| 1259 | 3-CF₃ | i-Propyl | i-Propyl | |
| 1260 | H | Et | i-propyl | |
| 1261 | 3-Cl | CH₂OEt | CHFCH₃ | |
| 1262 | H | CH₂OMe | Me | |
| 1263 | 3-F,5-Me | CF₃ | CHFCH₃ | |
| 1264 | 3,5-Me₂ | CH₂F | CHFCH₃ | |
| 1265 | 3,5-Me₂ | s-Butyl | CHFCH₃ | |
| 1266 | 3-F,5-OMe | CH₂-c-Butyl | i-Propyl | |
| 1267 | H | Me | CF(CH₃)₂ | |
| 1268 | 3-Me | CH₂OMe | CF(CH₃)₂ | |
| 1269 | 3-Me | Me | CF(CH₃)₂ | |
| 1270 | 3-F | c-Propyl | CF₂CHF₂ | |
| 1271 | 3,5-(OMe)₂ | CH₂-c-Butyl | CF₃ | |
| 1272 | 3-F,5-OMe | CH₂OEt | CHFCH₃ | |
| 1273 | H | H | Me | |
| 1274 | 3-Me | Me | CF₂CHF₂ | |
| 1275 | 3-Cl | Et | i-Propyl | |
| 1276 | 3-F | Et | CF(CH₃)₂ | |
| 1277 | H | Et | Et | |
| 1278 | 3-Me | Pentyl | CF₂CHF₂ | |
| 1279 | 3,5-F₂ | Me | CF₃ | |
| 1280 | 3-OMe,5-Cl | i-Butyl | i-propyl | |
| 1281 | 3-CF₃ | i-Butyl | CF₂CHF₂ | |
| 1282 | 3-Cl | Me | CF₂CHF₂ | |
| 1283 | H | c-Propyl | CF(CH₃)₂ | |
| 1284 | 3-Cl | c-Butyi | CF₂CHF₂ | |
| 1285 | 3-OEt | CHrC-BLityl | i-Propyl | |
| 1286 | H | Me | i-propyl | |
| 1287 | 3-Me | Me | CHFCH₃ | |
| 1288 | 3-F | H | CF(CH₃)₂ | |
| 1289 | 3-Me,4-F | Me | CF₂CHF₂ | |
| 1290 | 3-Me | Et | CHFCH₃ | |
| 1291 | 3-F | Et | i-propyl | |
| 1292 | H | c-Propyl | CF₂CHF₂ | |
| 1293 | 3-Cl | c-Propyl | CF(CH₃)₂ | |
| 1294 | H | s-Butyl | Me | |
| 1295 | 3-Me | c-Butyl | CHFCH₃ | |
| 1296 | 3-F | c-Butyl | CF(CH₃)₂ | |
| 1297 | 3-OMe | c-Pentyi | CHFCH₃ | |
| 1298 | 3,5-OMe | CH₂OMe | CHFCH₃ | |
| 1299 | 3-OMe,5-Cl H | CF(CH₃)₂ | | |
| 1300 | 3-OEt | CH₂OEt | CHFCH₃ | |
| 1301 | 3-F,5-Me | i-Butyl | CF₃ | |
| 1302 | 3-CF₃ | Me | CF(CH₃)₂ | |
| 1303 | 3-Me,4-F | Me | CF(CH₃)₂ | |
| 1304 | 3-Me | c-Propyl | CF₂CHF₂ | |
| 1305 | 3-OMe | Pentyl | i-propyl | |
| 1306 | 3-Cl,5-Me | c-Pentyl | CHFCH₃ | |
| 1307 | H | H | CHFCH₃ | resin |
| 1308 | 3-Cl | Me | CHFCH₃ | |
| 1309 | 3-Me | Et | i-Propyl | |
| 1310 | H | c-Propyl | i-propyl | |
| 1311 | 3-F | c-Propyl | CHFCH₃ | |
| 1312 | 3-Cl | c-Butyl | CHFCH₃ | |
| 1313 | 3,5-(OMe)₂ | CH₂-cPropyl | CF(CH₃)₂ | |
| 1314 | 3-OEt | i-Butyl | CF₃ | |
| 1315 | 3-Cl,5-Me | Hexyl | i-propyl | |
| 1316 | 3-F,5-Me | H | CF(CH₃)₂ | |
| 1317 | H | Me | CF₂CHF₂ | |
| 1318 | 3-Cl | c-Butyl | CF(CH₃)₂ | |
| 1319 | 3-OMe,5-Cl | CH₂F | i-Propyl | |
| 1320 | 3-Cl | Me | i-Propyl | |
| 1321 | 3-F | CH₂OEt | CHFCH₃ | |
| 1322 | 3-Cl | c-Butyl | i-Propyl | |
| 1323 | 3-OMe,5-Cl | CF₃ | CF₂CHF₂ | |
| 1324 | H | Et | CHFCH₃ | |
| 1325 | 3-F | c-Propyl | i-Propyl | |
| 1326 | 3,5-Me₂ | CF₃ | CF₂CHF₂ | |
| 1327 | 3-F,5-OMe | CH₂-c-Propyl | CF₃ | |
| 1328 | 3-F | Me | i-Propyl | |
| 1329 | H | c-Propyl | CHFCH₃ | |
| 1330 | 3-F | H | i-Propyl | |
| 1331 | H | H | Et | |
| 1332 | 3-Cl | Et | CHFCH₃ | |
| 1333 | 3-Me | c-Butyl | i-Propyl | |
| 1334 | 3,5-F₂ | n-Butyl | CF₂CHF₂ | |
| 1335 | 3-Cl,5-Me | n-Butyl | CF₂CHF₂ | |
| 1336 | 3-F | Me | CF₂CHF₂ | |
| 1337 | 3-Me,4-F | Me | CHFCH₃ | |
| 1338 | 3-Me | c-Propyl | CF(CH₃)₂ | |
| 1339 | 3-Cl | c-Propyl | i-propyl | |
| 1340 | 3-F | c-Butyl | CF₂CHF₂ | |
| 1341 | 3-OMe | n-Butyl | CF₂CHF₂ | |

TABLE 13
Compounds of the formula (Ip) (=formula (I), in which m = 0, n = 1,
Y = —O—(CH₂)₂—O—; R³ = H, R⁴ = H;
R⁵ = H; R⁷, R⁹, R¹⁰ are each H and
aryl = phenyl which is substituted as shown in the table)

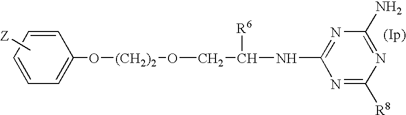

| No. | Z | R⁶ | R⁸ | Phys. data |
|---|---|---|---|---|
| 1342 | 3-F,5-Me | CF₃ | CHFCH₃ | |
| 1343 | 3,5-Me₂ | CH₂F | CHFCH₃ | |
| 1344 | H | Et | CF(CH₃)₂ | oil |
| 1345 | H | Et | CH(CH₃)₂ | oil |
| 1346 | H | Et | CHFCH₃ | oil |
| 1347 | 3-F | H | CF₂CHF₂ | |
| 1348 | 3-Cl | Pentyl | CF₂CHF₂ | |
| 1349 | 3-F | c-Butyl | CHFCH₃ | |
| 1350 | 3,5-Me₂ | t-Butyl | i-Propyl | |
| 1351 | 3-Cl,5-Me | Me | CHFCF₃ | |
| 1352 | 3,5-Me₂ | H | CHFCH₃ | |
| 1353 | 3,5-Me₂ | H | Et | |
| 1354 | 3,5-Me₂ | H | CF(CH₃)₂ | |
| 1355 | H | Et | CF₂CHF₂ | |
| 1356 | 3-F | c-Propyl | CF(CH₃)₂ | |
| 1357 | 3-Cl | Et | CF(CH₃)₂ | |
| 1358 | H | c-Butyl | CF₂CHF₂ | |
| 1359 | 3,5-F₂ | i-Propyl | 1-Propyl | |
| 1360 | 3-CF₃ | c-Pentyl | CF₃ | |
| 1361 | 3-Me | Me | 1-Propyl | |
| 1362 | 3-F | Me | CHFCH₃ | |
| 1363 | 3-F,5-OMe | CH₂OMe | CF₂CHF₂ | |
| 1364 | 3-Cl | Et | CF(CH₃)₂ | oil |
| 1365 | 3-Me | c-Propyl | 1-Propyl | |
| 1366 | 3-Me | Et | CF₂CHF₂ | |
| 1367 | 3-Cl | El | CF₂CHF₂ | |
| 1368 | H | c-Butyl | CF(CH₃)₂ | |
| 1369 | 3-Me | c-Butyl | CF(CH₃)₂ | |
| 1370 | 3,5-Me₂ | H | CH(CH₃)₂ | |
| 1371 | H | H | CF(CH₃)₂ | |
| 1372 | H | H | CH(CH₃)₂ | |
| 1373 | H | Et | Me | |
| 1374 | 3-Me,4-F | H | i-Propyl | |
| 1375 | 3-Me | c-Propyl | CHFCH₃ | |
| 1376 | 3-OMe | Me | CF₃ | |
| 1377 | 3,5-F₂ | c-Pentyl | CHFCH₃ | |
| 1378 | 3-CF₃ | i-Propyl | i-Propyl | |
| 1379 | 3-Cl | CH₂OEt | CHFCH₃ | |
| 1380 | H | CH₂OMe | Me | |
| 1381 | 3-F | c-Butyl | CF₂CHF₂ | |
| 1382 | 3-OMe | n-Butyl | CF₂CHF₂ | |
| 1383 | 3,5-Me₂ | s-Butyl | CHFCH₃ | |
| 1384 | 3-F,5-OMe | CH₂-c-Butyl | 1-Propyl | |
| 1385 | H | Me | CF(CH₃)₂ | oil |
| 1386 | 3-Me | CH₂OMe | CF(CH₃)₂ | |
| 1387 | 3-Me | Me | CF(CH₃)₂ | |
| 1388 | 3-F | c-Propyl | CF₂CHF₂ | |
| 1389 | 3,5-(OMe)₂ | CH₂-c-Butyl | CF₃ | |
| 1390 | 3-F,5-OMe | CH₂OEt | CHFCH₃ | |
| 1391 | H | H | CHFCH₃ | |
| 1392 | 3-Me | Me | CF₂CHF₂ | |
| 1393 | 3-Cl | Et | 1-Propyl | |
| 1394 | 3-F | Et | CF(CH₃)₂ | |
| 1395 | H | Et | Et | |
| 1396 | 3-Me | Pentyl | CF₂CHF₂ | |
| 1397 | 3,5-F₂ | Me | CF₃ | |
| 1398 | 3-OMe,5-Cl | i-Butyl | i-Propyl | |
| 1399 | 3-CF₃ | i-Butyl | CF₂CHF₂ | |
| 1400 | 3-Cl | Me | CF₂CHF₂ | |
| 1401 | H | c-Propyl | CF(CH₃)₂ | |
| 1402 | 3-Cl | c-Butyl | CF₂CHF₂ | |
| 1403 | 3-OEt | CH₂-c-Butyl | i-Propyl | |
| 1404 | H | Me | 1-Propyl | oil |
| 1405 | 3-Me | Me | CHFCH₃ | |
| 1406 | 3-F | H | CF(CH₃)₂ | |
| 1407 | 3-Me,4-F | Me | CF₂CHF₂ | |
| 1408 | 3-Me | Et | CHFCH₃ | |
| 1409 | 3-F | Et | 1-Propyl | |
| 1410 | H | c-Propyl | CF₂CHF₂ | |
| 1411 | 3-Cl | c-Propyl | CF(CH₃)₂ | |
| 1412 | H | s-Butyl | Me | |
| 1413 | 3-Me | c-Butyl | CHFCH₃ | |
| 1414 | 3-F | c-Butyl | CF(CH₃)₂ | |
| 1415 | 3-OMe | c-Pentyl | CHFCH₃ | |
| 1416 | 3,5-(OMe)₂ | CH₂OMe | CHFCH₃ | |
| 1417 | 3-OMe,5-Cl | H | CF(CH₃)₂ | |
| 1418 | 3-OEt | CH₂OEt | CHFCH₃ | |
| 1419 | 3-F,5-Me | t-Butyl | CF₃ | |
| 1420 | 3-CF₃ | Me | CF(CH₃)₂ | |
| 1421 | 3-Me,4-F | Me | CF(CH₃)₂ | |
| 1422 | 3-Me | c-Propyl | CF₂CHF₂ | |
| 1423 | 3-OMe | Pentyl | 1-Propyl | |
| 1424 | 3-Cl,5-Me | c-Pentyl | CHFCH₃ | |
| 1425 | H | H | CHFCH₃ | |
| 1426 | 3-Cl | Me | CHFCH₃ | |
| 1427 | 3-Me | Et | 1-Propyl | |
| 1428 | H | c-Propyl | i-Propyl | |
| 1429 | 3-F | c-Propyl | CHFCH₃ | |
| 1430 | 3-Cl | c-Butyl | CHFCH₃ | |
| 1431 | 3,5-(OMe)₂ | CH₂-cPropyl | CF(CH₃)₂ | |
| 1432 | 3-OEt | i-Butyl | CF₃ | |
| 1433 | 3-Cl,5-Me | Hexyl | 1-Propyl | |
| 1434 | 3-F,5-Me | H | CF(CH₃)₂ | |
| 1435 | H | Me | CF₂CHF₂ | |
| 1436 | 3-Cl | c-Butyl | CF(CH₃)₂ | |
| 1437 | 3-OMe,5-Cl | CH₂F | i-Propyl | |
| 1438 | 3-Cl | Me | i-Propyl | |
| 1439 | 3-F | CH₂OEt | CHFCH₃ | |
| 1440 | 3-Cl | c-Butyl | 1-Propyl | |
| 1441 | 3-OMe,5-Cl | CF₃ | CF₂CHF₂ | |
| 1442 | 3-F | c-Propyl | 1-Propyl | |
| 1443 | 3,5-Me₂ | CF₃ | CF₂CHF₂ | |
| 1444 | 3-F,5-OMe | CH₂-c-Propyl | CF₃ | |
| 1445 | 3-F | Me | i-Propyl | |
| 1446 | H | c-Propyl | CHFCH₃ | |
| 1447 | 3-F | H | i-Propyl | |
| 1448 | H | H | i-Propyl | |
| 1449 | 3-Cl | Et | CHFCH₃ | |
| 1450 | 3-Me | c-Butyl | i-Propyl | |
| 1451 | 3,5-F₂ | n-Butyl | CF₂CHF₂ | |
| 1452 | 3-Cl,5-Me | n-Butyl | CF₂CHF₂ | |
| 1453 | 3-F | Me | CF₂CHF₂ | |
| 1454 | 3-Me,4-F | Me | CHFCH₃ | |
| 1455 | 3-Me | c-Propyl | CF(CH₃)₂ | |
| 1456 | 3-Cl | c-Propyl | i-Propyl | |

### B. FORMULATION EXAMPLES

1. Dusting Agent

A dusting agent is obtained by mixing 10 parts by weight of a compound of the formula (I) and 90 parts by weight of talc as inert substance and comminuting the mixture in a hammer mill.

2. Dispersible Powder

A readily water-dispersible wettable powder is obtained by mixing 25 parts by weight of a compound of the formula (I), 64 parts by weight of kaolin-containing quartz as inert substance, 10 parts by weight of potassium ligninsulfonate and I part by weight of sodium oleoylmethyltaurinate as wetting agent and dispersant and grinding the mixture in a pinned disk mill.

3. Dispersion Concentrate

A readily water-dispersible concentrate is obtained by mixing 20 parts by weight of a compound of the formula (I), 6 parts by weight of alkylphenol polyglycol ether (®Triton X 207), 3 parts by weight of isotridecanol polyglycol ether (8 EO) and 71 parts by weight of paraffinic mineral oil (boiling range, for example, from about 255 to over 277° C.) and grinding the mixture in a ball mill to a fineness below 5 microns.

4. Emulsifiable Concentrate

An emulsifiable concentrate is obtained from 15 parts by weight of a compound of the formula (I), 75 parts by weight of cyclohexanone as solvent and 10 parts by weight of ethoxylated nonylphenol as emulsifier.

5. Water-Dispersible Granules

Water-dispersible granules are obtained by mixing

| | |
|---|---|
| 75 parts by weight | of a compound of the formula (I), |
| 10 parts by weight | of calcium ligninsulfonate, |
| 5 parts by weight | of sodium lauryl sulfate, |
| 3 parts by weight | of polyvinyl alcohol and |
| 7 parts by weight | of kaolin, |

grinding the mixture in a pinned disk mill and granulating the powder in a fluidized bed by spraying on water as granulation liquid.

Water-dispersible granules are also obtained by homogenizing and precomminuting

| | | |
|---|---|---|
| 25 | parts by weight | of a compound of the formula (I), |
| 5 | parts by weight | of sodium 2,2′-dinaphthylmethane-6,6′-disulfonate, |
| 2 | parts by weight | of sodium oleoylmethyltaurinate, |
| 1 | part | of polyvinyl alcohol, |
| 17 | parts | of calcium carbonate and |
| 50 | parts | of water, |

in a colloid mill, followed by grinding in a bead mill, and atomizing and drying the resulting suspension in a spray tower using a single-substance nozzle.

### C. BIOLOGICAL EXAMPLES

1. Pre-Emergence Action Against Weeds

Seeds of mono- and dicotyledonous weed plants are placed in sandy loam soil in cardboard pots and covered with soil. The novel compounds, formulated as wettable powders or emulsion concentrates, are then applied to the surface of the soil cover in the form of an aqueous suspension or emulsion at an application rate of from 600 to 800 l of water/ha (converted), in various dosages. After the treatment, the pots are placed in a greenhouse and kept under good growth conditions for the weeds. After the test plants have emerged after a trial period of 3 to 4 weeks, the damage to the plants or the negative effect on emergence is rated visually by comparison with untreated controls. As shown by the test results, the novel compounds have good herbicidal pre-emergence activity against a broad spectrum of weed grasses and broad-leaved weeds. For example, the compounds of Example Nos. 41, 42, 64, 76, 78, 120, 167, 183, 201, 207, 213, 220, 222, 228, 231, 245, 253, 281, 282, 297, 299, 300, 303, 305, 308, 311, 326, 329, 336, 339, 340, 347, 358, 368, 369, 370, 372, 376, 380, 381, 384, 394, 490, 501, 502, 510, 528, 531, 532, 553, 554, 582, 598, 605, 609, 618, 626, 636, 645, 681, 696, 698, 869, 870, 926, 930, 987, 1005, 1234, 1235, 1236, 1251, 1252, 1253, 1307, 1344, 1345, 1346, 1364, 1385 and 1404 show good and in most cases very good herbicidal activity against harmful plants such as Stellaria media, Amaranthus retroflexus, Sinapis avreuse, Avena fatura, Lolium multiflorum and Setaria viridis at an application rate of 3.0 kg or less of active substance per hectare.

2. Post-Emergence Action Against Weeds

Seeds of mono- and dicotyledonous weeds are placed in sandy loam soil in cardboard pots, covered with soil and cultivated in a greenhouse under good growth conditions. Two to three weeks after sowing, the test plants are treated at the three-leaf stage. The novel compounds, formulated as wettable powders or emulsion concentrates, are sprayed in various dosages onto the green parts of the plant at an application rate of from 600 to 8001 of water/ha (converted), and, after the test plants have remained in the greenhouse for 3 to 4 weeks under optimum growth conditions, the action of the preparations is rated visually by comparison with untreated controls. The novel compositions also have good post-emergence herbicidal activity against a broad spectrum of economically important weed grasses and broad-leaved weeds. For example, the compounds of Example Nos. 20, 23, 41, 42, 43, 46, 78, 80, 183, 184, 201, 207, 213, 220, 222, 228, 231, 245, 253, 281, 297, 299, 303, 305, 308, 311, 326, 336, 340, 347, 358, 368, 369, 370, 372, 376, 380, 381, 384, 490, 501, 502, 510, 528, 531, 532, 553, 554, 582, 598, 605, 609, 618, 626, 636, 645, 681, 696, 698, 869, 870, 926, 930, 987, 1005, 1234, 1235, 1236, 1251, 1252, 1253, 1307, 1344, 1345, 1346, 1364, 1385 and 1404 show good and in most cases very good herbicidal activity against harmful plants such as Stellaria media, Amaranthus retroflexus, Sinapis avreuse, Avena fatura, Lolium multiflorum and Setaria viridis, at an application rate of 3.0 kg or less of active substance per hectare.

3. Action on Weeds in Rice

Typical rice weeds such as Echinocloa crus galli and Cyperus iria are cultivated in a greenhouse under paddy rice conditions (dammed height of water: 2-3 cm). After the treatment, the test plants are set up in a greenhouse under optimum growth conditions and are maintained in this way under these conditions throughout the test period. About three weeks after application, evaluation is made by visual rating of the damage to the plants by comparison with untreated controls. The compounds according to the invention have very good herbicidal activity against harmful plants. For example, the compounds of Example Nos. 41, 42, 46, 120, 183, 201, 207, 213, 220, 222, 228, 231, 245, 253, 281, 299, 300, 303, 308, 311, 326, 336, 340, 347, 358, 368, 369, 370, 372, 376, 380, 381, 384 and 394 show very good herbicidal activity against Cyperus iria and Echinocloa crus-galli.

4. Tolerance by Crop Plants

In further greenhouse experiments, seeds of a relatively large number of crop plants and weeds are placed in sandy loam soil and covered with soil. Some of the pots are treated immediately as described under section 1, while the remainder are placed in a greenhouse until the plants have developed two to three true leaves, and then sprayed with various dosages of the novel substances of the formula (I), as described in section 2. Four to five weeks after the application, and after the plants have remained in the greenhouse, visual rating shows that the compounds according to the invention leave dicotyledonous crops such as, for examle, soya and sugar beet undamaged or almost undamaged when employed pre- and post-emergence, even when high dosages of active compound are used. Moreover, some substances also leave gramineous crops unharmed, for example barley, wheat and rice. Some of the compounds of the formula (I) display a high selectivity and are therefore suitable for controlling unwanted plant growth in agricultural crops.

1. A compound of the formula I, if appropriate also in its salt form, 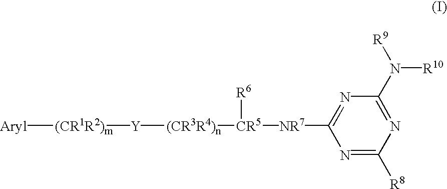
in which
aryl is an unsubstituted or substituted mono- or bicyclic aromatic radical having 5 to 14 ring atoms, 1, 2, 3 or 4 of which may in each case independently of one another originate from the group consisting of oxygen, sulfur and nitrogen;
—Y— is a divalent unit from the group consisting of —O—, —S—, —NR¹¹—, —NR²CONR¹³—, —CO₂—, —OCO₂—, —OCONR¹⁴—, —SO—, —SO₂—, —SO₂O—, —OSO₂O—, —SO₂NR⁴—, —O—NR¹¹—, —NR′—NR″—, in which R′ and R″ independently of one another are as defined for R¹⁴, and —(Y′—CR^{a}R^{b}—CR^{c}R^{d}); —Y″, in which Y′ and Y″ independently of one another are O, S, NH or N[(C₁-C₄)alkyl], R^{a}, R^{b}, R^{c} and R^{d} in each case independently of one another are H or (C₁-C₄)alkyl and i is an integer from 1 to 5, or a trivalent unit of the formula —O—N═,
m is 0, 1, 2, 3, 4 or 5,
n is an integer from 1 to 10, with the proviso that n is not 1 if m is zero and —Y— is —O—, —S—, —SO—, —SO₂— or —NR¹¹—;
R¹, R² in each case independently of one another are a radical from a group G1 which comprises hydrogen, (C₁-C₁₀)-alkyl, (C₂-C₈)-alkenyl, (C₂-C₈)-alkynyl, (C₁-C₁₀)-alkoxy, (C₃-C₈)-cycloalkyl, (C₃-C₈)-cycloalkoxy, aryl-(C₁-C₆)-alkyl and (C₃-C₈)-cycloalkyl-(C₁-C₆)-alkyl, where the cyclic moiety of the four last-mentioned radicals is in each case unsubstituted or substituted by one or more identical or different radicals from the group consisting of halogen, nitro, cyano, thiocyanato and —B—X¹, where —B— and X¹ are as defined below, and where the non-cyclic moiety of the eight last-mentioned radicals from the group G1 is in each case unsubstituted or substituted by one or more identical or different radicals from the group consisting of halogen, nitro, cyano, thiocyanato and —B—X², where X² is as defined below, and where the non-cyclic moiety of the radicals from the group G1 may in each case be interrupted by one or more identical or different heteroatoms from the group consisting of oxygen and sulfur,
R¹ and R² of a (CR¹R²) group form, together with the carbon atom which carries them, a carbonyl group, a group CR¹⁵R¹⁶ or a 3- to 6-membered ring which may contain one or two identical or different heteroatoms from the group consisting of oxygen, nitrogen and sulfur and which is unsubstituted or substituted by one or more identical or different radicals from the group consisting of halogen, nitro, cyano, thiocyanato and —B—X¹, or
two R¹ of two directly adjacent or not directly adjacent (CR¹R²) groups together with the carbon atoms which carry or link them form an unsubstituted or substituted 3- to 6-membered ring which may contain one or two identical or different heteroatoms from the group consisting of oxygen, nitrogen and sulfur and which is unsubstituted or substituted by one or more identical or different radicals from the group consisting of halogen, nitro, cyano, thiocyanato and —B—X¹ or
two R¹ of two directly adjacent (CR¹R²) groups together with the bond between the carbon atoms of the groups form a double bond, or two R¹ and two R² of two directly adjacent (CR¹R²) groups together with the bond between the carbon atoms of the groups form a triple bond or
R¹ is a linkage point for the double bond in the case where Y is a trivalent unit ═N—O— adjacent to a CR¹R² group,
R³, R⁴ in each case independently of one another are a radical of a group G2 which comprises hydrogen, (C₁-C₁₀)-alkyl, (C₂-C₈)-alkenyl, (C₂-C₈)-alkynyl, (C₁-C₁₀)-alkoxy, (C₁-C₁₀)-alkylthio, (C₁-C₁₀)-alkylsulfinyl, (C₁-C₁₀)-alkylsulfonyl, (C₃-C₈)-cycloalkyl, (C₃-C₈)-cycloalkoxy, aryl, aryl-(C₁-C₆)-alkyl, aryl-(C₁-C₆)-alkoxy, (C₃-C₈)-cycloalkyl-(C₁-C₆)-alkyl, (C₃-C₈)-cycloalkyl-(C₁-C₆)-alkoxy, (C₃-C₈)-cycloalkoxy-(C₁-C₆)-alkyl and (C₃-C₈)-cycloalkoxy-(C₁-C₆)-alkoxy, where the cyclic moiety of the nine last-mentioned radicals is in each case unsubstituted or substituted by one or more identical or different radicals from the group consisting of halogen, nitro, cyano, thiocyanato and —B—X¹, where —B— and X¹ are as defined below, and where the non-cyclic moiety of the sixteen last-mentioned radicals from the group G2 is in each case unsubstituted or substituted by one or more identical or different radicals from the group consisting of halogen, nitro, cyano, thiocyanato and —B—X², where X² is as defined below, and where the non-cyclic moiety of the radicals from the group G2 may in each case be interruped by one or more identical or different heteroatoms from the group consisting of oxygen and sulfur, or
R³ and R⁴ together with the carbon atom which carries them form a carbonyl group, a group CR¹⁵R¹⁶ or a 3- to 6-membered ring, which may contain one or two identical or different heteroatoms from the group consisting of oxygen, nitrogen and sulfur and which is unsubstituted or substituted by one or more identical or different radicals from the group consisting of halogen, nitro, cyano, thiocyanato and —B—X¹, or
two R³ of two directly adjacent or not directly adjacent (CR³R⁴) groups together with the carbon atoms which carry or link them form an unsubstituted or substituted 3- to 6-membered ring which may contain one or two identical or different heteroatoms from the group consisting of oxygen, nitrogen and sulfur and which is unsubstituted or substituted by one or more identical or different radicals from the group consisting of halogen, nitro, cyano, thiocyanato and —B—X¹, or
two R³ of two directly adjacent (CR³R⁴) groups together with the bond between the carbon atoms of the groups form a double bond, or two R³ and two R⁴ of two directly adjacent (CR³R⁴) groups together with the bond between the carbon atoms of the groups form a triple bond, or
R³ is a linkage point for the double bond in the case where Y is a trivalent unit —O—N═ adjacent to a CR³R⁴ group,
—B— is a direct bond or a divalent unit from the group consisting of —O—, —S—, —NR¹¹—, —NR¹²CONR¹³—, —CO₂—, —OCO₂—, —OCONR¹⁴—, —SO—, —SO₂—, —SO₂O—, —OSO₂O— and —SO₂NR¹⁴—;
X¹ is hydrogen, (C₁-C₈)-alkyl, (C₂-C₈)-alkenyl, (C₂-C₈)-alkynyl, (C₃-C₈)-cycloalkyl or heterocyclyl having 3 to 9 ring atoms, 1, 2 or 3 of which originate from the group consisting of nitrogen, oxygen and sulfur, and where the five last-mentioned radicals are unsubstituted or substituted by one or more identical or different halogen atoms;
X² is hydrogen or heterocyclyl having 3 to 9 ring atoms, 1, 2 or 3 of which originate from the group consisting of nitrogen, oxygen and sulfur, which is unsubstituted or substituted by one or more identical or different halogen atoms;
R⁵, R⁵ independently of one another are each a radical from the group G2, or
R³ and R⁵ of two directly adjacent or not directly adjacent (CR³R⁴) or (CR⁵R⁶) groups together with the carbon atoms which link them form an unsubstituted or substituted 3- to 6-membered ring which may contain one or two identical or different heteroatoms from the group consisting of oxygen, nitrogen and sulfur and which is unsubstituted or substituted by one or more identical or different radicals from the group consisting of halogen, nitro, cyano, thiocyanato and —B—X¹, or
R⁵ and R⁶ together with the carbon atom which carries them form a carbonyl group, a group CR¹⁵R¹⁶ or a 3- to 6-membered ring which may contain one or two identical or different heteroatoms from the group consisting of oxygen, nitrogen and sulfur and which is unsubstituted or substituted by one or more identical or different radicals from the group consisting of halogen, nitro, cyano, thiocyanato and —B—X¹, or
R⁶ is heterocyclyl;
R⁷ is hydrogen, amino, alkylcarbonyl, alkylamino or dialkylamino having in each case one to six carbon atoms in the alkyl radical, is an acyclic hydrocarbon or hydrocarbonoxy radical having in each case one to six carbon atoms, is a cyclic hydrocarbon or hydrocarbonoxy radical having in each case three to six carbon atoms or is heterocyclyl, heterocyclyloxy or heterocyclylamino having in each case three to six ring atoms and one to three hetero ring atoms from the group consisting of nitrogen, oxygen and sulfur, where each of the ten last-mentioned radicals is unsubstituted or substituted by one or more identical or different radicals from the group consisting of halogen, (C₁-C₄)-alkoxy, halogen-(C₁-C₄)-alkoxy, (C₁-C₄)-alkylthio, (C₂-C₄)-alkenyl, (C₂-C₄)-alkynyl, (C₂-C₄)-alkenyloxy, (C₂-C₄)-alkynyloxy, hydroxyl, amino, acylamino, alkylamino, dialkylamino, nitro, carboxyl, cyano, azido, (C₁-C₄)-alkoxycarbonyl, (C₁-C₄)-alkylcarbonyl, formyl, carbamoyl, mono- and di-(C₁-C₄)-alkylaminocarbonyl, (C₁-C₄)-alkylsulfinyl, halo-(C₁-C₄)-alkylsulfinyl, (C₁-C₄)-alkylsulfonyl, halo-(C₁-C₄)-alkylsulfonyl and, in the case of cyclic radicals, also (C₁-C₄)-alkyl and halo-(C₁-C₄)-alkyl;
R⁸ is (C₁-C₁₀)-alkyl, (C₂-C₈)-alkenyl, (C₂-C₈)-alkynyl, which are unsubstituted or substituted by one or more identical or different radicals from the group consisting of halogen, cyano, nitro, thiocyanato, hydroxyl, (C₁-C₄)-alkoxy, (C₁-C₄)-alkylthio, (C₁-C₄)-alkylsulfinyl, (C₁-C₄)-alkylsulfonyl, phenyl, (C₃-C₉)-cycloalkyl, (C₃-C₉)-cycloalkoxy, and is heterocyclyl having three to six ring atoms and one to three hetero ring atoms from the group consisting of oxygen, nitrogen and sulfur, which is unsubstituted or substituted by one or more identical or different radicals from the group consisting of halogen, amino, (C₁-C₄)-alkyl, (C₁-C₄)-alkoxy, halo-(C₁-C₄)-alkyl and halo-(C₁-C₄)-alkoxy,
is (C₃-C₈)-cycloalkyl, (C₃-C₈)-cycloalkoxy or a heterocyclyl radical having three to six ring atoms, where these three last-mentioned radicals are unsubstituted or substituted by one or more identical or different radicals from the group consisting of halogen, nitro, cyano, thiocyanato, (C₁-C₄)-alkyl, (C₁-C₄)-alkoxy, halo-(C₁-C₄)-alkyl and halo-(C₁-C₄)-alkoxy;
R⁹, R¹⁰ independently of one another are each hydrogen, amino, (C₁-C₁₀)-alkylcarbonyl, (C₁-C₁₀)-alkylamino, di-[(C₁-C₁₀)-alkyl]amino, (C₁-C₁₀)-alkyl, (C₃-C₈)-cycloalkyl, (C₁-C₁₀)-alkoxy, (C₃-C₈)-Cycloalkoxy, heterocyclyl, heterocyclyloxy or heterocyclylamino having in each case 3 to 6 ring atoms and 1 to 3 hetero ring atoms from the group consisting of oxygen, nitrogen and sulfur, where each of the ten last-mentioned radicals is unsubstituted or substituted, or
R⁹ and R¹⁰ together with the nitrogen atom which carries them form a heterocycle having a total of three to six ring atoms and from among these one to four hetero ring atoms, where the other hetero ring atoms which may be present in addition to the nitrogen atom that is present are selected from the group consisting of oxygen, nitrogen and sulfur and where this heterocycle is unsubstituted or substituted;
R¹¹ is hydrogen, amino, (C₁-C₁₀)-alkylamino, di-[(C₁-C₁₀)-alkyl]amino, (C₁-C₁₀)-alkyl, (C₃-C₈)-cycloalkyl, (C₃-C₈)-cycloalkyl-(C₁-C₆)-alkyl, (C₁-C₁₀)-alkoxy, (C₁-C₆)-alkoxy-(C₁-C₆)-alkoxy, (C₃-C₈)-cycloalkoxy, (C₁-C₁₀)-alkylcarbonyl, where the nine last-mentioned radicals are unsubstituted or substituted,
R¹², R¹³ independently of one another are each hydrogen, (C₁-C₁₀)-alkyl, (C₂-C₈)-alkenyl, (C₂-C₈)-alkynyl, phenyl, phenyl-(C₁-C₆)-alkyl, (C₃-C₈)-cycloalkyl, (C₃-C₈)-cycloalkyl-(C₁-C₆)-alkyl, where the cyclic moiety of the four last-mentioned radicals is in each case unsubstituted or substituted by one or more identical or different radicals from the group consisting of (C₁-C₄)-alkyl, halo-(C₁-C₄)-alkyl, (C₁-C₄)-alkoxy and halo-(C₁-C₄)-alkoxy, or
R¹² and R¹³ together with the N—CO—N group which carries them form a 5- to 8-membered ring which, in addition to the two abovementioned nitrogen atoms, may contain a further heteroatom from the group consisting of oxygen, nitrogen and sulfur and which is unsubstituted or substituted;
R¹⁴ is hydrogen or in each case unsubstituted or substituted (C₁-C₁₀)-alkyl or (C₃-C₁₀)-cycloalkyl and
R¹⁵, R¹⁶ independently of one another are each hydrogen, aryl, (C₁-C₁₀)-alkoxy, aryl-(C₁-C₆)-alkyl, (C₁-C₁₀)-alkyl, (C₁-C₁₀)-alkylthio, where the five last-mentioned radicals are unsubstituted or substituted, and where the aliphatic carbon skeleton of the three last-mentioned radicals may be interrupted by one or more identical or different heteroatoms from the group consisting of oxygen and sulfur, or
R¹⁵ and R¹⁶ together with the carbon atom which carries them form a 3- to 6-membered ring which may contain one or two identical or different heteroatoms from the group consisting of oxygen, nitrogen and sulfur and which is unsubstituted or substituted.

2. The compound as claimed in claim 1, wherein
aryl is an unsubstituted or substituted mono- or bicyclic aromatic radical having 5 to 10 ring atoms, 1, 2, 3 or 4 of which may in each case independently of one another originate from the group consisting of oxygen, sulfur and nitrogen;
—Y— is a divalent unit from the group consisting of —O—, —S—, —NR¹¹—, —NR¹²CONR¹³—, —CO₂—, —OCONR¹⁴—, —SO—, —SO₂—, —SO₂O—, —SO₂NR¹⁴—, —O—NR¹¹—, —NH—NH— and —O—CR^{a}R^{b}—CR^{c}R^{d}—O—, in which R^{a}, R^{b}, R^{c} and R^{d} independently of one another are each H, CH₃ or C₂H₅,
or else a trivalent unit of the formula —O—N═ and
R¹, R² independently of one another are each a radical from a group G3 which comprises hydrogen, (C₁-C₈)-alkyl, (C₂-C₆)-alkenyl, (C₂-C₆)-alkynyl, (C₁-C₆)-alkoxy, (C₃-C₈)-cycloalkyl, (C₃-C₈)-cycloalkoxy, aryl-(C₁-C₆)-alkyl and (C₃-C₈)-cycloalkyl-(C₁-C₆)-alkyl, where the cyclic moiety of the four last-mentioned radicals is in each case unsubstituted or substituted by one or more identical or different radicals from the group consisting of halogen, nitro, cyano and —B—X¹, where —B— and X¹ are as defined below, and where the non-cyclic moiety of the eight last-mentioned radicals from the group G3 is in each case unsubstituted or substituted by one or more identical or different radicals from the group consisting of halogen, nitro, cyano and —B—X², where X² is as defined below, and where the non-cyclic moiety of the radicals from the group G3 may in each case be interrupted by one or more identical or different heteroatoms from the group consisting of oxygen and sulfur, or
R¹ and R² in a (CR¹R²) group form, together with the carbon atom which carries them, a carbonyl group, a group CR¹⁵R¹⁶ or 3- to 6-membered ring which may contain one or two identical or different heteroatoms from the group consisting of oxygen, nitrogen and sulfur and which is unsubstituted or substituted by one or more identical or different radicals from the group consisting of halogen, nitro, cyano and —B—X¹, or
two R¹ of two directly adjacent or not directly adjacent (CR¹R²) groups form, together with the carbon atoms which carry or link them, an unsubstituted or substituted 3- to 6-membered ring which may contain one or two identical or different heteroatoms from the group consisting of oxygen, nitrogen and sulfur and which is unsubstituted or substituted by one or more identical or different radicals from the group consisting of halogen, nitro, cyano and —B—X¹, or
two R¹ of two directly adjacent (CR¹R²) groups together with the bond between the carbon atoms of the groups form a double bond, or two R¹ and two R² of two directly adjacent (CR¹R²) groups together with the bond between the carbon atoms of the groups form a triple bond or
R¹ is a linkage point for the double bond in the case where Y is a trivalent unit ═N—O— adjacent to a CR¹R² group, and
R³ and R⁴ independently of one another are each a radical from a group G4 which comprises hydrogen, (C₁-C₈)-alkyl, (C₂-C₆)-alkenyl, (C₂-C₆)-alkynyl, (C₁-C₆)-alkoxy, (C₃-C₈)-cycloalkyl, (C₃-C₈)-cycloalkoxy, aryl, aryl-(C₁-C₆)-alkyl, aryl-(C₁-C₆)-alkoxy, (C₃-C₈)-cycloalkyl-(C₁-C₆)-alkyl, (C₃-C₈)-cycloalkyl-(C₁-C₆)-alkoxy, (C₃-C₈)-cycloalkoxy-(C₁-C₆)-alkyl and (C₃-C₈)-cycloalkoxy-(C₁-C₆)-alkoxy, where the cyclic moiety of the nine last-mentioned radicals is in each case unsubstituted or substituted by one or more identical or different radicals from the group consisting of halogen, nitro, cyano, thiocyanato and —B—X¹, where —B— and X¹ are as defined below, and where the non-cyclic moiety of the thirteen last-mentioned radicals from the group G4 is in each case unsubstituted or substituted by one or more identical or different radicals from the group consisting of halogen, nitro, cyano, thiocyanato and —B—X², where X² is as defined below, and where the non-cyclic moiety of the radicals from the group G4 may in each case be interrupted by one or two identical or different heteroatoms from the group consisting of oxygen and sulfur, or
R³ and R⁴ together with the carbon atom which carries them form a carbonyl group, a group CR¹⁵R¹⁶ or a 3- to 6-membered ring, which may contain one or two identical or different heteroatoms from the group consisting of oxygen, nitrogen and sulfur and which is unsubstituted or substituted by one or more identical or different radicals from the group consisting of halogen, nitro, cyano and —B—X¹, or
two R³ of two directly adjacent or not directly adjacent (CR³R⁴) groups form, together with the carbon atoms which carry or link them, an unsubstituted or substituted 3- to 6-membered ring which may contain one or two identical or different heteroatoms from the group consisting of oxygen, nitrogen and sulfur and which is unsubstituted or substituted by one or more identical or different radicals from the group consisting of halogen, nitro, cyano and —B—X¹; or
two R³ of two directly adjacent (CR³R⁴) groups together with the bond between the carbon atoms of the groups form a double bond, or two R³ and two R⁴ of two directly adjacent (CR³R⁴) groups together with the bond between the carbon atoms of the groups form a triple bond or
R³ is a linkage point for the double bond in the case where Y is a trivalent unit ═N—O— adjacent to a CR³R⁴ group, and
—B— is a direct bond or a divalent unit from the group consisting of —O—, —S—, —NR¹¹—, —CO—, —CO₂—, —OCO₂—, —SO—, —SO₂—, —SO₂O— und —SO₂NR¹⁴—;
X¹ is hydrogen, (C₁-C₆)-alkyl, (C₂-C₆)-alkenyl, (C₂-C₆)-alkynyl, (C₃-C₆)-cycloalkyl or heterocyclyl having 3 to 6 ring atoms, 1, 2 or 3 of which originate from the group consisting of nitrogen, oxygen and sulfur, and where the five last-mentioned radicals are unsubstituted or substituted by one or more identical or different halogen atoms,
X² is hydrogen or heterocyclyl having 3 to 9 ring atoms, 1, 2 or 3 of which originate from the group consisting of nitrogen, oxygen and sulfur and which is unsubstituted or substituted by 1, 2 or 3 identical or different halogen atoms,
R⁵, R⁶ independently of one another are each a radical from the group G4, or
R³ and R⁵ of two directly adjacent or not directly adjacent (CR³R⁴)— or (CR⁵R⁶)— groups together with the carbon atoms which link them form an unsubstituted or substituted 3- to 6-membered ring which may contain one or two identical or different heteroatoms from the group consisting of oxygen, nitrogen and sulfur and which is unsubstituted or substituted by one or more identical or different radicals from the group consisting of halogen, nitro, cyano and —B—X¹, or
R⁵ and R⁶ together with the carbon atom which carries them form a carbonyl group, a group CR¹⁵R¹⁶ or a 3- to 6-membered ring which may contain one or two identical or different heteroatoms from the group consisting of oxygen, nitrogen and sulfur and which is unsubstituted or substituted by one or more identical or different radicals from the group consisting of halogen, nitro, cyano and —B—X¹, or
R⁶ is heterocyclyl and
R⁷ is hydrogen, amino, alkylcarbonyl, alkylamino or dialkylamino having in each case one to four carbon atoms in the alkyl radical, is an acyclic or cyclic hydrocarbon radical having in each case one to six carbon atoms, or is heterocyclyl having three to six ring atoms and one to three hetero ring atoms from the group consisting of nitrogen, oxygen and sulfur, where each of the six last-mentioned radicals is unsubstituted or substituted by one or more identical or different radicals from the group consisting of halogen, (C₁-C₄)-alkoxy, halo-(C₁-C₄)-alkoxy, (C₁-C₄)-alkylthio, (C₂-C₄)-alkenyl, (C₂-C₄)-alkynyl, (C₂-C₄)-alkenyloxy, (C₂-C₄)-alkynyloxy, hydroxyl, amino, alkylamino, dialkylamino, nitro, carboxyl, cyano, (C₁-C₄)-alkoxycarbonyl, (C₁-C₄)-alkylcarbonyl, formyl, carbamoyl, mono- and di-(C₁-C₄)-alkylaminocarbonyl, (C₁-C₄)-alkylsulfonyl, halo-(C₁-C₄)-alkylsulfonyl and, in the case of cyclic radicals, also (C₁-C₄)-alkyl and halo-(C₁-C₄)-alkyl,
R⁸ is (C₁-C₆)-alkyl, which is unsubstituted or substituted by one or more identical or different radicals from the group consisting of halogen, cyano, nitro, thiocyanato, hydroxyl, (C₁-C₄)-alkoxy, (C₁-C₄)-alkylthio, (C₁-C₄)-alkylsulfonyl, (C₂-C₄)-alkenyl, (C₂-C₄)-alkynyl, phenyl, (C₃-C₉)-cycloalkyl, (C₃-C₉)-cycloalkoxy and heterocyclyl having three to six ring atoms and one to three hetero ring atoms from the group consisting of oxygen, nitrogen and sulfur which is unsubstituted or substituted by one or more identical or different radicals from the group consisting of halogen, amino, (C₁-C₄)-alkyl, (C₁-C₄)-alkoxy, halo-(C₁-C₄)-alkyl and halo-(C₁-C₄)-alkoxy, is (C₃-C₈)-Cycloalkyl or (C₃-C₈)-cycloalkoxy, where the two last-mentioned radicals are unsubstituted or substituted by one or more identical or different radicals from the group consisting of halogen, nitro, cyano, (C₁-C₄)-alkyl, (C₁-C₄)-alkoxy, halo-(C₁-C₄)-alkyl and halo-(C₁-C₄)-alkoxy,
R⁹, R¹⁰ independently of one another are each hydrogen, amino, (C₁-C₆)-alkylcarbonyl, (C₁-C₆)-alkylamino, di-[(C₁-C₆)-alkyl]amino, (C₁-C₆)-alkyl, (C₃-C₈)-cycloalkyl, (C₁-C₆)-alkoxy, (C₃-C₈)-cycloalkoxy or heterocyclyl having 3 to 6 ring atoms and 1 to 3 hetero ring atoms from the group consisting of oxygen, nitrogen and sulfur, where each of the eight last-mentioned radicals is unsubstituted or substituted by one or more identical or different radicals from the group consisting of halogen, amino, hydroxyl and, in the case of cyclic radicals, also (C₁-C₄)-alkyl, (C₁-C₄)-alkoxy, halo-(C₁-C₄)-alkyl and halo-(C₁-C₄)-alkoxy, or
R⁹ and R¹⁰ together with the nitrogen atom which carries them form a heterocycle having a total of three to six ring atoms and among these one to four hetero ring atoms, where the other hetero ring atoms which may be present in addition to the nitrogen atom that is present are selected from the group consisting of oxygen, nitrogen and sulfur and where this heterocycle is unsubstituted or substituted by up to three identical or different radicals from the group consisting of halogen, amino, hydroxyl,
(C₁-C₄)-alkyl, (C₁-C₄)-alkoxy, halo-(C₁-C₄)-alkyl and halo-(C₁-C₄)-alkoxy; and
R¹¹ is hydrogen, amino, (C₁-C₆)-alkylamino, di-[(C₁-C₆)-alkyl]amino, (C₁-C₆)-alkyl, (C₃-C₈)-cycloalkyl, (C₃-C₈)-cycloalkyl-(C₁-C₆)-alkyl, (C₁-C₆)-alkoxy, (C₁-C₆)-alkoxy-(C₁-C₆)-alkoxy, (C₃-C₈)-cycloalkoxy, (C₁-C₆)-alkylcarbonyl, where the nine last-mentioned radicals are unsubstituted or substituted by one or more identical or different radicals from the group consisting of halogen, amino, hydroxyl and, in the case of cyclic radicals, also (C₁-C₄)-alkyl, (C₁-C₄)-alkoxy, halo-(C₁-C₄)-alkyl and halo-(C₁-C₄)-alkoxy,
R¹², R¹³ independently of one another are each hydrogen, (C₁-C₆)-alkyl, (C₂-C₆)-alkenyl, (C₂-C₆)-alkynyl, phenyl, phenyl-(C₁-C₆)-alkyl, (C₃-C₈)-cycloalkyl, (C₁-C₈)-cycloalkyl-(C₁-C₆)-alkyl, where the cyclic moiety of the four last-mentioned radicals is in each case unsubstituted or substituted by one or more identical or different radicals from the group consisting of (C₁-C₄)-alkyl, halo-(C₁-C₄)-alkyl, (C₁-C₄)-alkoxy and halo-(C₁-C₄)-alkoxy, or
R¹² and R¹³ together with the N—CO—N group which carries them form a 5- to 8-membered ring which, in addition to the two abovementioned nitrogen atoms, may contain a further heteroatom from the group consisting of oxygen, nitrogen and sulfur and which is unsubstituted or substituted by one or more identical or different radicals from the group consisting of halogen, (C₁-C₄)-alkyl and halo-(C₁-C₄)-alkyl; and
R¹⁴ is hydrogen or (C₁-C₆)-alkyl or (C₃-C₈)-cycloalkyl, each of which is unsubstituted or substituted by one or more identical or different radicals from the group consisting of halogen, phenyl, hydroxyl, (C₁-C₄)-alkoxy and halo-(C₁-C₄)-alkoxy and
R¹⁵ and R¹⁶ independently of one another are each hydrogen, phenyl, (C₁-C₆)-alkoxy, phenyl-(C₁-C₆)-alkyl, (C₁-C₆)-alkyl, (C₁-C₆)-alkylthio, where the five last-mentioned radicals are unsubstituted or substituted, and where the aliphatic carbon skeleton of the three last-mentioned radicals may be interrupted by one or more identical or different heteroatoms from the group consisting of oxygen and sulfur, or
R¹⁵ and R¹⁶ together with the carbon atom which carries them form a 3- to 6-membered ring which may contain one or two identical or different heteroatoms from the group consisting of oxygen, nitrogen and sulfur and which is unsubstituted or substituted by one or more identical or different radicals from the group consisting of halogen, (C₁-C₄)-alkyl and halo-(C₁-C₄)-alkyl.

3. The compound as claimed in claim 1, wherein
aryl is a mono- or bicyclic aromatic radical having 5 to 10 ring atoms, 1, 2, 3 or 4 of which may in each case independently of one another originate from the group consisting of oxygen, sulfur and nitrogen, which is unsubstituted or substituted by one or more identical or different radicals from the group consisting of halogen, hydroxyl, amino, formyl, (C₁-C₄)-alkoxycarbonyl, (C₁-C₄)-alkylcarbonyl, halo-(C₁-C₄)-alkoxycarbonyl, halo-(C₁-C₄)-alkylcarbonyl, (C₁-C₄)-alkyl, (C₂-C₄)-alkenyl, (C₂-C₄)-alkynyl, (C₁-C₄)-alkoxy, (C₁-C₄)-alkoxy-(C₁-C₄)-alkyl, halo-(C₁-C₄)-alkoxy-(C₁-C₄)-alkyl, halo-(C₁-C₄)-alkoxy-[halo-(C₁-C₄)-alkyl], (C₁-C₄)-alkoxy-[halo-(C₁-C₄)-alkyl], (C₃-C₆)-cycloalkyl, (C₃-C₆)-cycloalkyl-(C₁-C₄)-alkyl, (C₃-C₆)-cycloalkyl-[halo-(C₁-C₄)-alkyl], halo-(C₃-C₆)-cycloalkyl-[halo-(C₁-C₄)-alkyl], halo-(C₃-C₆)-cycloalkyl-(C₁-C₄)-alkyl, (C₁-C₆)-alkylamino, di-[(C₁-C₆)-alkyl]amino, (C₁-C₆)-alkylaminocarbonyl, aminosulfonyl, (C₁-C₆)-alkylaminosulfonyl, di-[(C₁-C₆)-alkyl]aminosulfonyl, halo-(C₁-C₄)-alkyl and halo-(C₁-C₄)-alkoxy,
—Y— is a divalent unit from the group consisting of —O—, —S—, —NR¹¹—, —CO₂—, —SO—, —SO₂—, —SO₂O—, —SO₂NR¹⁴—, —NH—CO—NH—, —OCONH—, —O—NH— and —OCH₂CH₂—O— or a trivalent unit —O—N═,
R¹, R² independently of one another are in each case a radical from a group G5 which comprises hydrogen, (C₁-C₆)-alkyl, (C₂-C₄)-alkenyl, (C₂-C₄)-alkynyl, (C₁-C₆)-alkoxy, (C₃-C₆)-cycloalkyl, (C₃-C₆)-cycloalkoxy, aryl-(C₁-C₆)-alkyl and (C₃-C₆)-cycloalkyl-(C₁-C₆)-alkyl, where the cyclic moiety of the four last-mentioned radicals is in each case unsubstituted or substituted by one or more identical or different radicals from the group consisting of halogen, nitro, cyano and —B—X¹, where —B— and X¹ are as defined below, and where the non-cyclic moiety of the eight last-mentioned radicals from the group G5 is in each case unsubstituted or substituted by one or more identical or different radicals from the group consisting of halogen, nitro, cyano and —B—X², where X² is as defined below, and where the non-cyclic moiety of the radicals from the group G5 may in each case be interrupted by one or more identical or different heteroatoms from the group consisting of oxygen and sulfur, or
R¹ and R² of a (CR¹R²) group form, together with the carbon atom that carries them, a carbonyl group, a group CR¹⁵R¹⁶ or 3- to 6-membered ring which may contain one or two identical or different heteroatoms from the group consisting of oxygen, nitrogen and sulfur and which is unsubstituted or substituted by up to four identical or different radicals from the group consisting of halogen, nitro, cyano and —B—X¹, or
two R¹ of two directly adjacent or not directly adjacent (CR¹R²) groups form, together with the carbon atoms that carry or link them, an unsubstituted or substituted 3- to 6-membered ring which may contain one or two identical or different heteroatoms from the group consisting of oxygen, nitrogen and sulfur and which is unsubstituted or substituted by up to four identical or different radicals from the group consisting of halogen, nitro, cyano and —B—X¹, or
two R¹ of two directly adjacent (CR¹R²) groups together with the bond between the carbon atoms of the groups form a double bond, or two R¹ and two R² of two directly adjacent (CR¹R²) groups together with the bond between the carbon atoms of the groups form a triple bond, or
R¹ is a linkage point for the double bond in the case where Y is a trivalent unit ═N—O— adjacent to a CR¹R² group,
R³, R⁴ independently of one another are in each case a radical from a group G6 which comprises hydrogen, (C₁-C₆)-alkyl, (C₂-C₄)-alkenyl, (C₂-C₄)-alkynyl, (C₁-C₆)-alkoxy, (C₃-C₈)-cycloalkyl, (C₃-C₈)-cycloalkoxy, aryl, aryl-(C₁-C₄)-alkyl, aryl-(C₁-C₄)-alkoxy, (C₃-C₈)-cycloalkyl-(C₁-C₄)-alkyl, (C₃-C₈)-cycloalkyl-(C₁-C₄)-alkoxy, (C₃-C₈)-cycloalkoxy-(C₁-C₄)-alkyl and (C₃-C₈)-cycloalkoxy-(C₁-C₄)-alkoxy, where the cyclic moiety of the nine last-mentioned radicals is in each case unsubstituted or substituted by one or more identical or different radicals from the group consisting of halogen, nitro, cyano and —B—X¹, where —B— and X¹ are as defined below, and where the non-cyclic moiety of the thirteen last-mentioned radicals from the group G6 is in each case unsubstituted or substituted by one or more identical or different radicals from the group consisting of halogen, nitro, cyano and —B—X², where X² is as defined below, and where the non-cyclic moiety of the radicals from the group G6 may in each case be interrupted by a heteroatom from the group consisting of oxygen and sulfur, or
R³ and R⁴ together with the carbon atom that carries them form a carbonyl group, a group CR¹⁵R¹⁶ or a 3- to 6-membered ring, which may contain one or two identical or different heteroatoms from the group consisting of oxygen, nitrogen and sulfur and which is unsubstituted or substituted by up to four identical or different radicals from the group consisting of halogen, nitro, cyano and —B—X¹, or
two R³ of two directly adjacent or not directly adjacent (CR³R⁴) groups form, together with the carbon atoms that carry or link them, an unsubstituted or substituted 3- to 6-membered ring which may contain one or two identical or different heteroatoms from the group consisting of oxygen, nitrogen and sulfur and which is unsubstituted or substituted by up to four identical or different radicals from the group consisting of halogen, nitro, cyano and —B—X¹, or
two R³ of two directly adjacent (CR³R⁴) groups together with the bond between the carbon atoms of the groups form a double bond, or two R³ and two R⁴ of two directly adjacent (CR³R⁴) groups together with the bond between the carbon atoms of the groups form a triple bond or
R³ is a linkage point for the double bond in the case where Y is a trivalent unit ═N—O— adjacent to a CR³R⁴ group, and
—B— is a direct bond or a divalent unit from the group consisting of —O—, —S—, —NR¹¹—, —CO—, —CO₂—, —SO—, —SO₂— and —SO₂NR⁴—,
X¹ is hydrogen, (C₁-C₆)-alkyl, (C₂-C₄)-alkenyl, (C₂-C₄)-alkynyl, (C₃-C₆)-cycloalkyl or heterocyclyl having 3 to 6 ring atoms, 1, 2 or 3 of which originate from the group consisting of nitrogen, oxygen and sulfur, and where the five last-mentioned radicals are unsubstituted or substituted by up to five identical or different halogen atoms,
X² is hydrogen or heterocyclyl having 3 to 6 ring atoms, 1, 2 or 3 of which originate from the group consisting of nitrogen, oxygen and sulfur, which is unsubstituted or substituted by 1, 2 or 3 identical or different halogen atoms,
R⁵, R⁶ independently of one another are each a radical of the group G6, or
R³ and R⁵ of two directly adjacent or not directly adjacent (CR³R⁴)— or (CR⁵R⁶)— groups form, together with the carbon atoms which link them, an unsubstituted or substituted 3- to 6-membered ring which may contain one or two identical or different heteroatoms from the group consisting of oxygen, nitrogen and sulfur and which is unsubstituted or substituted by up to four identical or different radicals from the group consisting of halogen, nitro, cyano and —B—X¹, or
R⁵ and R⁶ together with the carbon atom which carries them form a carbonyl group, a group CR¹⁵R¹⁶ or a 3- to 6-membered ring which may contain one or two identical or different heteroatoms from the group consisting of oxygen, nitrogen and sulfur and which is unsubstituted or substituted by one or more identical or different radicals from the group consisting of halogen, nitro, cyano, thiocyanato and —B—X¹, or
R⁶ is heterocyclyl and
R⁷ is hydrogen, amino, alkylcarbonyl, alkylamino or dialkylamino having in each case one to four carbon atoms in the alkyl radical, or is an acyclic or cyclic hydrocarbon radical having in each case one to six carbon atoms, where each of the five last-mentioned radicals is unsubstituted or substituted by one or more identical or different radicals from the group consisting of halogen, (C₁-C₄)-alkoxy, halo-(C₁-C₄)-alkoxy, (C₂-C₄)-alkenyl, (C₂-C₄)-alkynyl, (C₂-C₄)-alkenyloxy, (C₂-C₄)-alkynyloxy, hydroxyl, amino, (C₁-C₄)-alkylamino, di-[(C₁-C₄)-alkyl]amino, nitro, carboxyl, cyano, (C₁-C₄)-alkoxycarbonyl, (C₁-C₄)-alkylcarbonyl, formyl, carbamoyl, (C₁-C₄)-alkylsulfonyl, halo-(C₁-C₄)-alkylsulfonyl and, in the case of cyclic radicals, also (C₁-C₄)-alkyl and halo-(C₁-C₄)-alkyl,
R⁸ is (C₁-C₆)-alkyl which is unsubstituted or substituted by one or more identical or different radicals from the group consisting of halogen, cyano, hydroxyl, (C₁-C₄)-alkoxy, (C₁-C₄)-alkylthio, (C₁-C₄)-alkylsulfonyl, (C₂-C₄)-alkenyl, (C₂-C₄)-alkynyl, phenyl, (C₃-C₉)-Cycloalkyl, (C₃-C₉)-cycloalkoxy, is (C₃-C₈)-cycloalkyl or (C₃-C₈)-cycloalkoxy, where the two last-mentioned radicals are substituted or substituted by one or more identical or different radicals from the group consisting of halogen, nitro, cyano, (C₁-C₄)-alkyl, (C₁-C₄)-alkoxy, halo-(C₁-C₄)-alkyl and halo-(C₁-C₄)-alkoxy,
R⁹, R¹⁰ independently of one another are each hydrogen, amino, (C₁-C₄)-alkylcarbonyl, (C₁-C₄)-alkylamino, di-(C₁-C₄)-alkylamino, (C₁-C₄)-alkyl, (C₃-C₈)-cycloalkyl, (C₁-C₄)-alkoxy, (C₃-C₈)-cycloalkoxy or heterocyclyl having 3 to 6 ring atoms and 1 to 3 hetero ring atoms from the group consisting of oxygen, nitrogen and sulfur, where each of the eight last-mentioned radicals is unsubstituted or substituted by up to four identical or different radicals from the group consisting of halogen, amino, hydroxyl and, in the case of cyclic radicals, also (C₁-C₄)-alkyl, (C₁-C₄)-alkoxy, halo-(C₁-C₄)-alkyl and halo-(C₁-C₄)-alkoxy, or
R⁹ and R¹⁰ together with the nitrogen atom which carries them form a heterocycle having a total of three to six ring atoms and from among these one to four hetero ring atoms, where the other hetero ring atoms which may be present in addition to the nitrogen atom that is present are selected from the group consisting of oxygen, nitrogen and sulfur and where this heterocycle is unsubstituted or substituted;
R¹¹ is hydrogen, amino, (C₁-C₄)-alkylamino, di-(C₁-C₄)-alkylamino, (C₁-C₄)-alkyl, (C₃-C₈)-cycloalkyl, (C₃-C₈)-cycloalkyl-(C₁-C₄)-alkyl, (C₁-C₄)-alkoxy, (C₁-C₄)-alkoxy-(C₁-C₄)-alkoxy, (C₃-C₈)-cycloalkoxy, (C₁-C₄)-alkylcarbonyl, where the nine last-mentioned radicals are unsubstituted or substituted by one or more identical or different halogen atoms and, in the case of cyclic radicals, also (C₁-C₄)-alkyl, (C₁-C₄)-alkoxy, halo-(C₁-C₄)-alkyl and halo-(C₁-C₄)-alkoxy,
R¹⁴ is hydrogen or (C₁-C₄)-alkyl or (C₃-C₆)-cycloalkyl, each of which is optionally substituted by one or more identical or different radicals from the group consisting of halogen, (C₁-C₄)-alkoxy and halo-(C₁-C₄)-alkoxy and
R¹⁵, R¹⁶ independently of one another are each hydrogen, phenyl, (C₁-C₄)-alkoxy, phenyl-(C₁-C₄)-alkyl, (C₁-C₄)-alkyl, (C₁-C₄)-alkylthio, where the five last-mentioned radicals are unsubstituted or substituted by one or more identical or different halogen atoms and, in the case of cyclic radicals, also by one or more identical or different radicals from the group consisting of halogen, (C₁-C₄)-alkyl and halo-(C₁-C₄)-alkyl, or
R¹⁵ and R¹⁶ together with the carbon atom which carries them form a 3- to 6-membered ring which may contain one or two identical or different heteroatoms from the group consisting of oxygen, nitrogen and sulfur and which is unsubstituted or substituted by up to four identical or different radicals from the group consisting of halogen, (C₁-C₄)-alkyl and halo-(C₁-C₄)-alkyl.

4. The compound as claimed in claim 1, wherein
aryl is phenyl, naphthyl, pyridinyl, pyrimidinyl, furanyl, benzofuranyl, pyrrolyl, oxazolyl, thiazolyl, imidazolyl, oxadiazolyl or thienyl, each of which is unsubstituted or substituted by one or more identical or different radicals from the group consisting of halogen, hydroxyl, amino, formyl, (C₁-C₄)-alkoxycarbonyl, (C₁-C₄)-alkylcarbonyl, (C₁-C₄)-alkyl, (C₂-C₄)-alkenyl, (C₂-C₄)-alkynyl, (C₁-C₄)-alkoxy, (C₁-C₄)-alkylamino, di-[(C₁-C₄)-alkyl]amino, aminocarbonyl, (C₁-C₄)-alkylaminocarbonyl, di-[(C₁-C₄)-alkyl]aminocarbonyl, aminosulfonyl, (C₁-C₄)-alkylaminosulfonyl, di-[(C₁-C₄)-alkyl]aminosulfonyl, halo-(C₁-C₄)-alkyl, halo-(C₁-C₄)-alkoxy, (C₁-C₂)-alkylthio, (C₁-C₂)-alkylsulfinyl, (C₁-C₂)-alkylsulfonyl, halo-(C₁-C₂)-alkylthio, halo-(C₁-C₂)-alkylsulfinyl and halo-(C₁-C₂)-alkylsulfonyl,
—Y— is a divalent unit from the group consisting of —O—, —S—, —NR¹¹—, —CO₂—, —SO—, —SO₂—, —NH—CO—NH—, —O—CONH—, —O—NH— and —OCH₂CH₂O— or a trivalent unit of the formula —O—N═,
m is 0, 1, 2 or 3,
n is 1, 2, 3, 4 or 5,
R¹, R² independently of one another are each a radical from a group G7 which comprises (C₁-C₄)-alkyl, (C₂-C₄)-alkenyl, (C₂-C₄)-alkynyl, (C₁-C₄)-alkoxy, hydrogen, (C₃-C₆)-cycloalkyl, where the last-mentioned radical is unsubstituted or substituted by one to four identical or different radicals from the group consisting of halogen and —B—X¹, and where the four first-mentioned radicals from the group G7 are unsubstituted or substituted by one or more identical or different radicals from the group consisting of halogen and —B—X², where X² is as defined below, or
R¹ and R² of a (CR¹R²) group form together with the carbon atom which carries them a carbonyl group, or
two R¹ of two directly adjacent or not directly adjacent (CR¹R²) groups form, together with the carbon atoms which carry or link them, a 3-, 5- or 6-membered ring which may contain up to two heteroatoms from the group consisting of oxygen, sulfur and nitrogen and which is unsubstituted or substituted by up to four identical or different radicals from the group consisting of halogen and —B—X¹, or
R¹ is a linkage point for the double bond in the case where Y is a trivalent unit ═N—O— which is adjacent to a CR¹R² group, and
R³, R⁴ independently of one another are each a radical from a group G8 which comprises hydrogen, (C₁-C₄)-alkyl, (C₂-C₄)-alkenyl, (C₂-C₄)-alkynyl, (C₁-C₆)-alkoxy, (C₃-C₆)-cycloalkyl, (C₃-C₆)-cycloalkoxy, aryl, aryl-(C₁-C₂)-alkyl, aryl-(C₁-C₂)-alkoxy, (C₃-C₆)-cycloalkyl-(C₁-C₂)-alkyl and (C₃-C₈)-cycloalkoxy-(C₁-C₄)-alkyl, where the cyclic moiety of the seven last-mentioned radicals is in each case unsubstituted or substituted by up to four identical or different radicals from the group consisting of halogen, nitro, cyano and —B—X¹, where —B— and X¹ are as defined below, and where the non-cyclic moiety of the eleven last-mentioned radicals from the group G8 is in each case unsubstituted or substituted by one or more identical or different radicals from the group consisting of halogen, nitro, cyano and —B—X², where X² is as defined below, or
R³ and R³ of a (CR³R⁴) group form together with the carbon atom which carries them a carbonyl group, or
two R³ of two directly adjacent or not directly adjacent (CR³R⁴) groups form, together with the carbon atoms which carry or link them, a 3-, 5- or 6-membered ring which may contain up to two heteroatoms from the group consisting of oxygen, sulfur and nitrogen and which is unsubstituted or substituted by up to four identical or different radicals from the group consisting of halogen and —B—X¹, or
R³ is a linkage point for the double bond in the case where Y is a trivalent unit ═N—O— which is adjacent to a CR³R⁴ group, and
—B— is a direct bond or a divalent unit from the group consisting of —O—, —S—, —NR¹¹—, —CO— and —CO₂—;
X¹ is hydrogen, (C₁-C₄)-alkyl, (C₂-C₄)-alkenyl, (C₂-C₄)-alkynyl or (C₃-C₆)-cycloalkyl, the four last-mentioned radicals of which are unsubstituted or substituted by up to five identical or different halogen atoms,
X² is hydrogen or heterocyclyl having 3 to 6 ring atoms, 1, 2 or 3 of which originate from the group consisting of nitrogen, oxygen and sulfur and which is unsubstituted or substituted by one or more identical or different halogen atoms,
R⁵ is hydrogen, (C₁-C₄)-alkyl, halo-(C₁-C₄)-alkyl or (C₃-C₆)-cycloalkyl, or
R³ and R⁵ of two directly adjacent or not directly adjacent (CR³R⁴)— or (CR⁵R⁶) groups form, together with the carbon atoms which link them, a 3-, 5- or 6-membered ring which may contain up to two heteroatoms from the group consisting of oxygen, sulfur and nitrogen and which is unsubstituted or substituted by up to four identical or different radicals from the group consisting of halogen and —B—X¹, and
R⁶ is hydrogen, (C₁-C₄)-alkyl, (C₂-C₄)-alkenyl, (C₂-C₄)-alkynyl, (C₁-C₄)-alkoxy, (C₃-C₆)-cycloalkyl, (C₃-C₆)-cycloalkyl-(C₁-C₄)-alkyl, (C₃-C₆)-cycloalkoxy-(C₁-C₄)-alkyl, (C₃-C₆)-cycloalkoxy, (C₁-C₄)-alkoxy-(C₁-C₄)-alkyl or halo-(C₁-C₄)-alkyl,
R⁷ is hydrogen, amino or (C₁-C₄)-alkyl,
R⁸ is (C₁-C₄)-alkyl, halo-(C₁-C₄)-alkyl, (C₃-C₆)-cycloalkyl, halo-(C₃-C₆)-cycloalkyl or (C₃-C₆)-cycloalkyl-(C₁-C₄)-alkyl,
R⁹, R¹⁰ independently of one another are each hydrogen, amino, formyl or (C₁-C₄)-alkyl, or
R⁹ and R¹⁰ together with the nitrogen atom which carries them form a heterocycle having three to six ring atoms and one to two hetero ring atoms, where the other hetero ring atoms which may be present in addition to the nitrogen atom which is present are selected from the group consisting of oxygen and nitrogen,
R¹¹ is hydrogen, amino, (C₁-C₄)-alkylamino, di-[(C₁-C₄)-alkyl]amino, (C₁-C₄)-alkyl or (C₃-C₆)-cycloalkyl, where the five last-mentioned radicals are unsubstituted or substituted by one or more identical or different halogen atoms, and
R¹⁴ is hydrogen or (C₁-C₄)-alkyl.

5. A process for preparing a compound of the formula I or a salt thereof as claimed in claim 1, which comprises
a) reacting a compound of the formula (II)
R⁸-Fu (II)
in which Fu is a functional group from the group consisting of carboxylic ester, carboxylic orthoester, carbonyl chloride, carboxamide, carboxylic anhydride and trichloromethyl
with a biguanidide of the formula (III) or an acid addition salt thereof 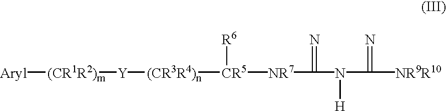
or
b) reacting a compound of the formula (IV) 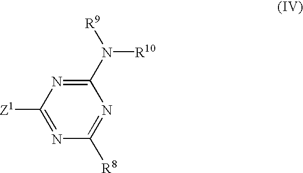
in which Z¹ is an exchangeable radical or a leaving group, for example chlorine, trichloromethyl, (C₁-C₄)-alkylsulfonyl and unsubstituted or substituted phenyl-(C₁-C₄)-alkylsulfonyl or (C₁-C₄)-alkylphenylsulfonyl, with a suitable amine of the formula (V) or an acid addition salt thereof 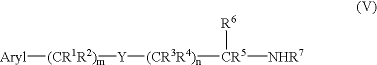
where in the formulae (II), (III), (IV) and (V) the radicals R¹ to R¹⁰, Y and n and m are as defined under formula (I).

6. A herbicidal or plant growth-regulatory composition, which comprises one or more compounds of the formula I or salts thereof as claimed in claim 1, if appropriate in a mixture with formulation auxiliaries.

7. A method for controlling undesirable plants or for regulating the growth of plants, wherein an effective amount of one or more compounds of the formula I or salts thereof as claimed in claim 1 is applied to the plants or to the location of the undesirable vegetation or to the area under cultivation.

8. The method as claimed in claim 7, wherein the compounds of the formula I or salts thereof are employed for controlling undesirable plants or for regulating the growth of plants in crops of useful plants.

9. The method as claimed in claim 8, wherein the useful plants are transgenic useful plants.
